(19) United States
(12) Patent Application Publication
(10) Pub. No.: US 2021/0038559 A1
(43) Pub. Date: Feb. 11, 2021

(54) CANNABINOIDS AND DERIVATIVES FOR PROMOTING IMMUNOGENICITY OF TUMOR AND INFECTED CELLS

(71) Applicants: Thomas Richard GADEK, Park City, UT (US); Patrick William GRAY, Seattle, WA (US); Wilfrid Arthur JEFFRERIES, Surrey (CA); PASCAL BIOSCIENCES INC., Vancouver (CA)

(72) Inventors: Thomas Richard GADAK, Park City, UT (US); Patrick William GRAY, Seattle, WA (US); Wilfred Arthur JEFFERIES, Surrey, British Columbia (CA)

(21) Appl. No.: 16/963,894

(22) PCT Filed: Jan. 22, 2019
(86) PCT No.: PCT/US2019/014566

§ 371 Date: Jul. 22, 2020

Related U.S. Application Data
(60) Provisional application No. 62/620,017, filed on Jan. 22, 2018.

(51) Int. Cl. A61K 31/352; A61K 31/05; A61K 35/17; A61K 39/395; A61K 38/21; A61K 38/20; A61P 35/00

(52) U.S. Cl. CPC A61K 31/352; A61K 31/05; A61K 35/17; A61P 35/00; A61K 38/212; A61K 38/2013; A61K 39/3955

(57) ABSTRACT

This invention provides processes and compositions for enhancing the immunogenicity of tumor or infected cells by increasing expression of major histocompatibility complex (MHC) class I surface molecules. Tumor cells often circumvent immune recognition by reducing or eliminating MHC expression. The lack of MHC on their surface allows tumor cells to evade immune detection and enables uncontrolled growth. Similarly, certain viral or microbial infections result in MHC class I downregulation and the resulting immune evasion. With a unique reporter assay, we have determined that some cannabinoid compounds and structural analogs can increase MHC class I expression on tumor cells grown in cell culture. The increase of tumor and infected cell MHC class I expression will enable detection and destruction by cytotoxic T-lymphocyte cells. The process and compositions increase the immunogenicity of the target cells, e.g. tumor or infected cells, to enhance their destruction by cytotoxic lymphocytes. When administered in combination, such compositions may enhance the activity of immune-oncology and anti-infectious disease agents.

### CROSS REFERENCES TO RELATED APPLICATIONS

This application claims the benefit of U.S. Provisional Application No. 62/620,017, filed on Jan. 22, 2018, which is hereby incorporated by reference in its entirety and for all purposes as if fully set forth herein.

### FIELD OF THE INVENTION

This invention relates to pharmaceutical compositions and uses thereof in medical treatments. More specifically it relates to compositions and medical treatments for enhancing the immunogenicity of selected cells in a patient's body, thereby rendering the cells more susceptible to recognition and elimination by the body's immune system.

### BACKGROUND OF THE INVENTION

The cytotoxic T-lymphocyte (CTL) response is a major component of the immune system, active in immune surveillance and destruction of infected or malignant cells expressing foreign or altered antigens on their surface. The ligand of the antigen-specific T-cell receptor is a complex made up of a peptide fragment of a foreign antigen bound to a major histocompatibility complex (MHC) molecule. Cytotoxic T lymphocytes recognize peptide bound to MHC Class I molecules, which are normally expressed at the cell surface as ternary complexes which include a peptide portion (de la Roche et al., Nat. Rev. Immunol. 16:421-432, 2016). Formation of the ternary complex involves transport into the lumen of the endoplasmic reticulum of peptides generated by protein degradation in the cytoplasm.

Loss of tumor MHC class I expression, an immune escape strategy that is aimed to avoid T-cell recognition, is commonly found in malignant cells. Similarly, certain viral and microbial infections can cause MHC class I downregulation. The ability to induce upregulation of MHC class I surface expression is a critical step in the induction of an immune response for the destruction of diseased cells. Cannabinoids have demonstrated biological and pharmacological affects and have been shown to have a direct cytotoxic effect on tumor cells; however, there are no publications that demonstrate MHC induction by cannabinoids.

Accordingly, there is a need for agents that can increase the recognition of tumor or infectious agent antigens through increased MHC class I expression in cancer and infected cells and thereby improve the ability of the immune system to target such cells for destruction. Two genes located in the MHC region have been identified and implicated in the transport of peptides from the cytoplasm, namely TAP-1 and TAP-2. U.S. Pat. No. 6,361,770, Jefferies et. al., issued Mar. 26, 2002, teaches a method of enhancing the expression of MHC Class I molecules on surfaces of target cells, by introducing into the target cell nucleic acid sequences encoding and expressing TAP-1 or TAP-2. The expression in the target cells of TAP-1 or TAP-2 enhances the presentation of MHC class I surface molecules on the target cells, so that they can be detected and eliminated by CTLs of the immune system. The method is particularly useful in connection with tumor cells which have a deficiency in proteasome components so that they have less than normal TAP expression and consequently do not express sufficient MHC class I surface molecules to be recognized by CTLs. With in situ expression of augmented TAP from the added nucleic acid sequences, the target cells are brought under the recognition and action of the CTLs of the immune system.

### BRIEF SUMMARY OF THE INVENTION

According to a first aspect of the present invention, there is provided a process of enhancing the immunogenicity of cells by increasing the presentation of MHC class I surface molecules for detection by CTLs which comprises administering to the cells an effective amount of a bioacceptable substance that promotes enhanced MHC class I surface expression on the cells.

According to a second aspect, the invention provides a pharmaceutical composition for administration to a subject, such as a human or an animal, for example a companion or commercial animal, to enhance MHC class I surface expression on cells thereof. The composition comprises a bio-acceptable substance that promotes induction of MHC class I expression, to cause enhanced MHC class I surface expression on the cells. The composition further comprises a suitable adjuvant or carrier.

A further aspect of the present invention provides use in the preparation or manufacture of a composition and a suitable adjuvant or carrier for administration to a subject suffering from a disorder to cause enhanced MHC class I surface expression on the cells.

Various embodiments of this invention relate to use of a bio-acceptable substance, a cannabinoid or cannabinoid derivative thereof, to enhance antigen presentation on cancer cells.

The cancer cells may be present in a subject, and the subject may be one that is not immunocompromised.

Various embodiments of this invention relate to a composition for use in enhancing an immune response against cancer, said composition comprising a cannabinoid or cannabinoid derivative and a suitable adjuvant or carrier.

Various embodiments of this invention relate to use of a cannabinoid or cannabinoid derivative in preparation of a medicament for enhancing an immune response against cancer.

Various embodiments of this invention relate to use of a cannabinoid or cannabinoid derivative in preparation of a medicament for enhancing an immune response against cancer in subjects treated concurrently with immune activating agents such as checkpoint inhibitors, coactivating receptor agonists, and cancer vaccines. The enhanced efficacy of the activated immune response may be driven by elevated MHC class I surface expression.

Some embodiments of this invention relate to use of a cannabinoid or cannabinoid derivative to enhance antigen presentation on virally infected cells. The virally infected cells may be present in a subject.

Some embodiments of this invention relate to use of a cannabinoid or cannabinoid derivative to enhance antigen presentation on cells infected with an intracellular pathogen. The cells infected with an intracellular pathogen may be present in a subject.

Some embodiments of this invention relate to use of a cannabinoid or cannabinoid derivative in preparation of a medicament for enhancing an immune response against an intracellular pathogen.

Some embodiments of this invention relate to use of a cannabinoid or cannabinoid derivative in preparation of a medicament for enhancing an immune response against an intracellular pathogen in patients treated concurrently with antiviral compounds.

### BRIEF DESCRIPTION OF THE FIGURES

FIG. 1A-1B. Cannabigerol stimulates MHC class I expression in the mouse A9 Lewis lung carcinoma (FIG. 1A) and the human COLO 205 colorectal carcinoma (FIG. 1B) cell lines. Cells were treated for 24 hours with 21.2 μM (A) or 25 and 50 μM (FIG. 1B) cannabigerol. Cell surface MHC class I expression was measured by flow cytometry.

FIG. 2A-2F. Dose-response stimulation of MHC class I expression in the human COLO 205 cell line by five distinct cannabinoids (FIG. 2A-2E) or interferon-γ (FIG. 2F). Cells were treated for 24 hours and cell surface MHC class I expression was measured by flow cytometry.

FIG. 3A-3B. Cell surface MHC class I expression was measured by flow cytometry (mean fluorescence intensity (MFI)) over 48 hours after treatment with IFN-γ. MHC-I downregulation is reversible in the majority of human and mouse cancer cell lines.

FIG. 4. Cell surface MHC class I expression was measured by flow cytometry (MFI). Endogenous cannabinoids, represented by AEA (N-arachidonoylethanolamine; anandamide) and 2-AG (2-Arachidonoylglycerol), do not induce MHC-I expression in COLO 205 cells.

FIG. 5. MHC-I expression (MFI) is induced over time in CBD-treated COLO 205 cells.

FIG. 6. Cannabigerol treatment of A9 cells for 48 hours at various doses increases MHC class I protein expression, as determined by flow cytometry. MHC class I protein upregulation was found to be statistically significant between DMSO treated cells and Cannabigerol treated cells at a concentration of 0.055 μM while using an ordinary one-way ANOVA. IFN γ treatment was at 5.832×10⁻⁶ in two mL of media.

FIG. 7A-7B. Number of live lymphocytes observed within the tumor of tumor bearing mice (n=8 for PBS negative control and Cannabigerol treatments, n=6 for TSA positive control). A9 tumors were present in mice for 7 days before intraperitoneal treatment, and mice were sacrificed 4-5 days after treatment. Cannabigerol upregulation of MHC-I expression attracts increased number of lymphocytes which can infiltrate the tumor.

FIG. 8. A proliferation experiment using CD8⁺ T cells from SIINFEKL-primed OT1 mice in response to SIINFEKL peptide presented on MHC class I of A9 cells. A9 cells were treated with 0.055 μM of Cannabigerol, 5.832×10⁻⁶ nM of IFN gamma positive control. The negative control is named “CFSE and SIINFEKL CD8⁺ T cells”. T cells were labeled with CFSE proliferation dye, which is reduced within the progeny cells as the generations progress.

FIG. 9 MHC Class I expression of A9 cells associated with CD8⁺ T cells from SIINFEKL-primed OT. A9 cells were treated with 0.055 μM of Cannabigerol, or 5.832×10⁴ nM of IFN gamma.

FIG. 10A-10E show the cytokines and other markers that have increased or decreased expression after treatment with cannabinoids, as compared to interferon gamma. FIG. 10A) Inflammatory markers; FIG. 10B) Cell growth markers; FIG. 10C) Leukocyte migration markers; FIG. 10D) Cell growth and differentiation markers; FIG. 10E) summary of markers.

### DESCRIPTION OF THE PREFERRED EMBODIMENTS

The embodiments listed below may be presented in numbered form for convenience and in ease and clarity of reference in referring back to multiple embodiments.

According to the preferred embodiments, the bio-acceptable substance is a cannabinoid, such as cannabigerol, a cannabinoid mimetic, or a cannabinoid derivative thereof, or pharmaceutical compositions made therefrom. According to specific preferred embodiments, the substance is a novel compound with structural relationship to a cannabinoid. According to additional specific preferred embodiments, the substance is a novel compound with additional modifications beyond structural relationship to a cannabinoid. Such modifications may enable improved biochemical or pharmaceutical properties.

While it is not intended that the present invention should be limited by any particular theory of action or biochemical mechanisms by which it is believed to operate, the following is offered and postulated for a better understanding of the invention and its practice.

Cannabinoids are a class of diverse chemical compounds that act on cannabinoid receptors and on additional cellular targets such as other G protein-coupled receptors, ion channels, transporters, and enzymes in the brain and other tissues (Soderstrom et al., Frontiers Pharmacol. 8:1-28, 2017). Phytocannabinoids are found in Cannabis sativa and some other plants, and some of these natural products have demonstrated pharmacological properties beyond their commonly known psychoactive properties. The endocannabinoids, such as anandamide, are produced naturally in the body and act as natural ligands for cannabinoid receptors (Devane et al., Science 258:1946-1949, 1992). Synthetic cannabinoids are manufactured artificially, have activity on cannabinoid receptors, and most have structural similarity to natural cannabinoids. Perhaps the most notable cannabinoid is tetrahydrocannabinol (THC), the primary psychoactive compound in Cannabis. More than 100 different cannabinoids have been isolated from Cannabis.

MHC class I antigens are found on nearly all nucleated cells of the body. The primary function of this class of MHC molecules is to display (or present) peptide fragments of intracellular proteins to CTLs. Based on this display, CTLs will ignore healthy cells and attack those displaying MHC-bound foreign or otherwise abnormal peptides, including disease-associated peptides (antigens) such as cancer antigens. Thus, the surface expression of MHC class I molecules plays a crucial role in determining the susceptibility of target cells to CTLs.

Many cancerous cells display down-regulated MHC class I cell surface expression (see, for example, Wang et al., J. Biol. Chem. 283:3951-3959, 2008; Chang et al., Keio J. Med. 52:220-229, 2003; Zagzag et al., Lab Invest. 85:328-341, 2005; Hewitt, Immunol. 110:163-169, 2003; Garrido et al., Curr. Opin. Immunol. 39:44-51, 2016). Reduced MHC-I expression can result at least in part from the down-regulation of multiple factors such as transporters (for example, TAP-1, TAP-2), proteasome components (LMP), and other accessory proteins involved in the antigen presentation and processing pathway. This characteristic may allow cancerous cells to evade immune surveillance and thereby provide a survival advantage against immune activity otherwise designed to eliminate the cells (Leone et al., J. Natl. Cancer Inst. 105:1172-1187, 2015; Hulpke and Tampe, Trends Biochem. Sci. 38:412-420, 2013).

In addition to cancerous cells, virally infected cells also may display altered MHC class I expression. Viruses such as human cytomegalovirus, Kaposi's sarcoma-associated herpes virus, human immunodeficiency virus, and others have evolved the capacity to modulate the class I antigen-presenting pathway through a number of mechanisms. These include increasing the endocytic rate of MHC class I complexes, blocking recognition of the complex by the T cell receptor of the CTL, destabilizing or interfering with the assembly of the MHC class I-peptide complex, expressing structural homologs of the MHC class I molecule that compete for antigenic peptide binding in the endoplasmic reticulum, and retaining the complex in the endoplasmic reticulum or golgi (see, for example, Christiansen et al., Current Opinion Immunol. 36:54-60, 2015; Liu et al., Int. J. Biochem. Cell Biol. 41:503-506, 2009; Wagner et al., J. Immunol. 180:19-24, 2008; Lilley and Plough, Immunol. Rev. 207: 126-144, 2005). Viruses can also evade immune detection by expressing viral MHC class I homologs that engage inhibitory receptors or inhibit activating receptors on natural killer (NK) cells, and by interfering with the MHC class II pathway (Wilkinson et al., J. Clin. Virol. 41:206-212, 2008; Liu et al., Int. J. Biochem. Cell Biol. 41:503-506, 2009). Bacterial and fungal pathogens also may use similar as well as distinct organism-specific mechanisms to evade the immune system (Goldberg et al., Microbiol. Spectrum 2:21-24, 2013; Kaparakis-Liaskos and Ferrero, Nat. Rev. Immunol. 15:375-387, 2015; Maksymowych and Kane, Microbes Infect. 2:199-211, 2000).

In one aspect, the invention provides a method wherein the cannabinoid compound or derivative thereof is administered with an additional therapeutic agent to said subject. For example, depending on the disorder being treated, the additional therapeutic agent is a cancer therapy, an antiviral therapy, or an antimicrobial therapy.

In a particular embodiment, the additional therapeutic agent is an agent that can be used to treat a cancer. The compounds of the present invention may also be used in combination with radiation therapy, hormone therapy, surgery and immunotherapy, which therapies are well known to those skilled in the art.

For example, an additional therapeutic agent can be selected from antineoplastic agents, anti-angiogenic agents, chemotherapeutic agents and peptidyl cancer therapy agents. These may include both cytotoxic agents or cytostatic agents. Cytotoxic agents are those agents that kill all cells. For example, cytotoxic agents can be classified as antineoplastic agents. Cytostatic agents include agents that slow or stop the growth of cells, including cancer cells, without killing them. These agents may cause tumors to stop growing and spreading without causing them to shrink in size.

In yet another embodiment, the antineoplastic agents are selected from antibiotic-type agents, alkylating agents, antimetabolite agents, hormonal agents, immunological agents, interferon-type agents, kinase inhibitors, miscellaneous agents and combinations thereof. It is noted that the additional therapeutic agents may be a traditional small organic chemical molecules or can be macromolecules such as a proteins, antibodies, peptibodies, DNA, RNA or fragments of such macromolecules.

Examples of specific therapeutic agents that can be used in combination with one or more compounds of the present invention include: atezolizumab, pembrolizumab, ipilimumab, methotrexate, tamoxifen, fluorouracil, 5-fluorouracil, hydroxyurea, mercaptopurine, cisplatin, carboplatin, daunorubicin, doxorubicin, etoposide, vinblastine, vincristine, paclitaxel, thioguanine, idarubicin, dactinomycin, imatinib, gemcitabine, altretamine, asparaginase, bleomycin, capecitabine, carmustine, cyclophosphamide, cytarabine, docetaxel, idarubicin, ifosfamide, irinotecan, fludarabine, mitosmycin, mitoxantrone, topotecan, vinoreibine, adriamycin, mithramycin, imiquimod, alemtuzmab, exemestane, bevacizumab, cetuximab, azacitidine, clofarabine, decitabine, desatinib, dexrazoxane, docetaxel, epirubicin, oxaliplatin, erlotinib, raloxifene, fulvestrant, letrozole, gefitinib, gemtuzumab, trastuzumab, gefitinib, ixabepilone, lapatinib, lenalidomide, aminolevulinic acid, temozolomide, nelarabine, sorafenib, nilotinib, pegaspargase, pemetrexed, rituximab, dasatinib, thalidomide, bexarotene, temsirolimus, bortezomib, vorinostat, capecitabine, zoledronic acid, anastrozole, sunitinib, aprepitant and nelarabine, or pharmaceutically acceptable salts thereof.

In one aspect, the invention provides a method where the additional therapeutic agent is an immune activating agent, i.e. a therapeutic that activates the immune response. Examples include checkpoint inhibitors, co-activating receptor agonists, and cancer-focused or pathogen-focused vaccines.

In one aspect, the invention provides a method where the additional therapeutic agent is a checkpoint inhibitor. Examples include CTLA and PD-1 pathway inhibitors.

In one aspect, the invention provides a method where the additional therapeutic agent is a PD-1 pathway inhibitor. As used herein “PD-1 pathway inhibitor” includes, but is not limited to, PD-1 binding agents, PD-L1 binding agents and PD-L2 binding agents. PD-1 binding agents include antibodies that specifically bind to PD-1. PD-L1 and PD-L2 binding agents include antibodies that specifically bind to PD-L1 and/or PD-L2, as well as soluble PD-1 polypeptides that specifically bind to PD-L1 and/or PD-L2.

In some embodiments, PD-1 pathway inhibitor is a PD-1-binding agent, for example an anti-PD-1 antibody. In some embodiments, the PD-1 pathway inhibitor is a PD-L1-binding agent, for example, an anti-PD-L1 antibody. In some embodiments, the PD-1 pathway inhibitor is a PD-L2-binding agent, for example an anti-PD-L2 antibody. In further embodiments, the PD-L1-binding agent is a soluble PD-1 polypeptide, for example, a PD-1-Fc fusion polypeptide capable of binding to PD-L1. In further embodiments, the PD-L2-binding agent is a soluble PD-1 polypeptide, for example, a PD-1-Fc fusion polypeptide capable of binding to PD-L2.

Anti-human-PD-1 antibodies (or VH and/or VL domains derived therefrom) suitable for use in the invention can be generated using methods well known in the art. Alternatively, art recognized anti-PD-1 antibodies can be used. For example, monoclonal antibodies 5C4 (referred to herein as Nivolumab or BMS-936558), 17D8, 2D3, 4H1, 4A11, 7D3, and 5F4, described in WO 2006/121 168 can be used. Other known PD-1 antibodies include lambrolizumab (MK-3475) described in WO 2008/156712, and AMP-514 described in WO 2012/145493. Further known PD-1 antibodies and other PD-1 inhibitors include those described in, for example, WO 2009/014708, WO 03/099196, WO 2009/114335 and WO 2011/161699, which are herein incorporated by reference. In one embodiment, the anti-PD-1 antibody is REGN2810. In one embodiment, the anti-PD-1 antibody is PDR001. Another known anti-PD-1 antibody is pidilizumab (CT-011).

In one embodiment, the anti-PD-1 antibody is nivolumab. Nivolumab (also known as “OPDIVO®”; formerly designated 5C4, BMS-936558, MDX-1106, or ONO-4538) is a fully human IgG4 (S228P) PD-1 checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., Cancer Immunol Res. 2(9):846-56 (2014)). In another embodiment, the anti-PD-1 antibody or fragment thereof cross-competes with nivolumab. In other embodiments, the anti-PD-1 antibody or fragment thereof binds to the same epitope as nivolumab. In certain embodiments, the anti-PD-1 antibody has the same CDRs as nivolumab.

Human monoclonal antibodies (HuMAbs) that bind specifically to PD-1 with high affinity have been disclosed in U.S. Pat. Nos. 8,008,449 and 8,779,105. Other anti-PD-1 mAbs have been described in, for example, U.S. Pat. Nos. 6,808,710, 7,488,802, 8, 168,757 and 8,354,509, and PCT Publication No. WO 2012/145493, which are herein incorporated by reference. In some embodiments, the anti-PD-1 antibody has been demonstrated to exhibit one or more of the following characteristics: (a) binds to human PD-1 with a K_{D} of 1×10^{″7} M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and (j) inhibits tumor cell growth in vivo. Anti-PD-1 antibodies useful for the present invention include mAbs that bind specifically to human PD-1 and exhibit at least one, at least two, at least three, at least four, or at least five of the preceding characteristics. Anti-PD-1 antibodies that exhibit one or more of these characteristics have been disclosed in U.S. Pat. Nos. 8,008,449, 8,779,105, 6,808,710, 7,488,802, 8, 168,757 and 8,354,509, and PCT Publication No. WO 2012/145493, which are herein incorporated by reference. In another embodiment, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587, which are herein incorporated by reference. In another embodiment, the antibody is cemiplimab (LIBTAYO®).

In some embodiments, the anti-PD-1 antibody or fragment thereof cross-competes with pembrolizumab. In some embodiments, the anti-PD-1 antibody or fragment thereof binds to the same epitope as pembrolizumab. In certain embodiments, the anti-PD-1 antibody has the same CDRs as pembrolizumab. In another embodiment, the anti-PD-1 antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587. Pembrolizumab has been approved by the FDA for the treatment of relapsed or refractory melanoma.

In other embodiments, the anti-PD-1 antibody or fragment thereof cross-competes with MEDI0608. In still other embodiments, the anti-PD-1 antibody or fragment thereof binds to the same epitope as MEDI0608. In certain embodiments, the anti-PD-1 antibody has the same CDRs as MEDI0608. In other embodiments, the anti-PD-1 antibody is MEDI0608 (formerly AMP-514), which is a monoclonal antibody. MED10608 is described, for example, in U.S. Pat. No. 8,609,089.

In certain embodiments, the first antibody is an anti-PD-1 antagonist. One example of the anti-PD-1 antagonist is AMP-224, which is a B7-DC Fc fusion protein. AMP-224 is discussed in U.S. Publ. No. 2013/0017199.

In other embodiments, the anti-PD-1 antibody or fragment thereof cross-competes with BGB-A317. In some embodiments, the anti-PD-1 antibody or fragment thereof binds the same epitope as BGB-A317. In certain embodiments, the anti-PD-1 antibody has the same CDRs as BGB-A317. In certain embodiments, the anti-PD-1 antibody is BGB-A317, which is a humanized monoclonal antibody. BGB-A317 is described in U.S. Publ. No. 2015/0079109.

In some embodiments, the antibody is pidilizumab (CT-011), which is an antibody previously reported to bind to PD-1 but which is believed to bind to a different target, pidilizumab is described in U.S. Pat. No. 8,686,119 or WO 2013/014668.

In certain embodiments, the antibodies that cross-compete for binding to human PD-1 with, or bind to the same epitope region of human PD-1 as, nivolumab are mAbs. For administration to human subjects, these cross-competing antibodies can be chimeric antibodies, or humanized or human antibodies. Such chimeric, humanized or human mAbs can be prepared and isolated by methods well known in the art.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/019846, WO 2017/024465, WO 2017/025016, WO 2017/132825, and WO 2017/133540 each of which is incorporated by reference in its entirety.

In some embodiments, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001(TAIZHOU JUNSHI PHARMA; see Si-Yang Liu et al., J. Hematol. Oncol. 70: 136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 70: 136 (2017)), TSR-042 (Tesaro Biopharmaceutical; also known as ANBO11; see WO2014/179664), GLS-010 (Wuxi/Harbin Gloria Pharmaceuticals; also known as WBP3055; see Si-Yang Liu et al., J. Hematol. Oncol. 70:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), and 11B1308 (Innovent; see WO 2017/024465, WO 2017/025016, WO 2017/132825, and WO 2017/133540).

Anti-PD-1 antibodies useful for the compositions of the disclosed invention also include antigen-binding portions of the above antibodies. It has been amply demonstrated that the antigen-binding function of an antibody can be performed by fragments of a full-length antibody. Examples of binding fragments encompassed within the term “antigen-binding portion” of an antibody include (i) a Fab fragment, a monovalent fragment consisting of the YL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains, and (iv) a Fv fragment consisting of the Vz, and YH domains of a single arm of an antibody.

Anti-PD-1 antibodies usable in the disclosed methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some embodiments, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

Anti-PD-1 antibodies suitable for use in the disclosed methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and upregulating the immune system. In certain embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain embodiments, the antibody is a humanized antibody. In other embodiments, the antibody is a human antibody. Antibodies of an IgG1, IgG2, IgG3 or IgG4 isotype can be used.

In other embodiments, the anti-PD-1 antibody is pembrolizumab. In other embodiments, the anti-PD-1 antibody is chosen from the human antibodies 17D8, 2D3, 4H1, 4A 11, 7D3 and 5F4 described in U.S. Pat. No. 8,008,449. In still other embodiments, the anti-PD-1 antibody is MEDI0608 (formerly AMP-514), AMP-224, or BGB-A317.

In embodiments, the anti-PD-1 antibody is a bispecific antibody.

In certain embodiments, the present application encompasses use of an anti-PD-L1 antibody as the PD-1 pathway inhibitor. In one embodiment, the anti-PD-L1 antibody inhibits the binding of PD-L1 receptor, i.e., PD-1 to its ligand PD-L1.

Anti-human-PD-L1 antibodies (or VH and/or VL domains derived therefrom) suitable for use in the invention can be generated using methods well known in the art. Alternatively, art recognized anti-PD-L1 antibodies can be used. For example, human anti-PD-L1 antibodies disclosed in U.S. Pat. No. 7,943,743 can be used. Such anti-PD-L1 antibodies include 3G10, 12A4 (also referred to as BMS-936559), 10A5, 5F8, 10H10, 1B12, 7H1, 11E6, 12B7, and 13G4. In some embodiments, the anti-PD-L1 antibody is atezolizumab (Tecentriq or RG7446) (see, e.g., Herbst et al. (2013) J Clin Oncol 31(suppl):3000. Abstract; U.S. Pat. No. 8,217,149), durvalumab (Imfinzi or MEDI4736) (Khleif (2013) In: Proceedings from the European Cancer Congress 2013; September 27-Oct. 1, 2013; Amsterdam, The Netherlands. Abstract 802), avelumab (Bavencio). Other art recognized anti-PD-L1 antibodies which can be used include those described in, for example, U.S. Pat. Nos. 7,635,757 and 8,217,149, U.S. Publication No. 2009/0317368, and PCT Publication Nos. WO 2011/066389 and WO 2012/145493, which are herein incorporated by reference. Antibodies that compete with any of these art-recognized antibodies or inhibitors for binding to PD-L1 also can be used. Examples of anti-PD-L1 antibodies useful in the methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a KD of 1×10-7 M or less, as determined by surface plasmon resonance using a Biacore biosensor system; (b) increase T-cell proliferation in a Mixed Lymphocyte Reaction (MLR) assay; (c) increase interferon-γ production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present invention include monoclonal antibodies that bind specifically to human PD-L and exhibit at least one, in some embodiments, at least five, of the preceding characteristics.

In certain embodiments, the anti-PD-L1 antibody is BMS-936559 (formerly 12A4 or MDX-1105) (see, e.g., U.S. Pat. No. 7,943,743; WO 2013/173223). In other embodiments, the anti-PD-L1 antibody is MPDL3280A (also known as RG7446 and atezolizumab) (see, e.g., Herbst et al. 2013 J Clin Oncol 31(suppl):3000; U.S. Pat. No. 8,217,149), MEDI4736 (Khleif, 2013, In: Proceedings from the European Cancer Congress 2013; September 27-Oct. 1, 2013. Amsterdam, The Netherlands. Abstract 802), or MSB0010718C (also called Avelumab; see US 2014/0341917). In certain embodiments, antibodies that cross-compete for binding to human PD-L1 with, or bind to the same epitope region of human PD-L as the above-references PD-L1 antibodies are mAbs. For administration to human subjects, these cross-competing antibodies can be chimeric antibodies, or can be humanized or human antibodies. Such chimeric, humanized or human mAbs can be prepared and isolated by methods well known in the art. In certain embodiments, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO2017/034916), and CK-301 (Checkpoint Therapeutics; see Gorelik et al., AACR:Abstract 4606 (April 2016)).

In certain embodiments, the PD-L1 antibody is atezolizumab (TECENTRIQ®). Atezolizumab is a fully humanized IgG1 monoclonal anti-PD-L1 antibody.

In certain embodiments, the PD-L antibody is durvalumab (IMFINZI™). Durvalumab is a human IgG kappa monoclonal anti-PD-L antibody.

In certain embodiments, the PD-L1 antibody is avelumab (BAVENCIO®). Avelumab is a human IgG lambda monoclonal anti-PD-L antibody.

Anti-PD-L1 antibodies usable in the disclosed methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of the reference antibody, e.g., atezolizumab and/or avelumab, by virtue of their binding to the same epitope region of PD-L. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain embodiments, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the methods of the disclosed invention also include antigen-binding portions of the above antibodies. It has been amply demonstrated that the antigen-binding function of an antibody can be performed by fragments of a full-length antibody. Anti-PD-L1 antibodies suitable for use in the disclosed methods or compositions are antibodies that bind to PD-L1 with high specificity and affinity, block the binding of PD-1, and inhibit the immunosuppressive effect of the PD-1 signaling pathway. In any of the compositions or methods disclosed herein, an anti-PD-L antibody includes an antigen-binding portion or fragment that binds to PD-L1 and exhibits the functional properties similar to those of whole antibodies in inhibiting receptor binding and up-regulating the immune system. In certain embodiments, the anti-PD-L1 antibody or antigen-binding portion thereof cross-competes with atezolizumab, durvalumab, and/or avelumab for binding to human PD-L1.

In certain embodiments, the present application encompasses use of an anti-CTLA-4 antibody. In one embodiment, the anti-CTLA-4 antibody binds to and inhibits CTLA-4. In some embodiments, the anti-CTLA-4 antibody is ipilimumab (YERVOY), tremelimumab (ticilimumab; CP-675,206), AGEN-1884, or ATOR-1015.

In certain embodiments, the checkpoint inhibitor is a CTLA-4 antagonist, a CD80 antagonist, a CD86 antagonist, a Tim-3 antagonist, a TIGIT antagonist, a CD20 antagonist, a CD96 antagonist, a CD27 agonist, CD28 agonist, a CD122 agonist, an OX40 agonist, an ICOS agonist, an IDO antagonist, a STING antagonist, a GARP antagonist, a CD40 antagonist, an A2aR antagonist, a CEACAM1 (CD66a) antagonist, a CEA antagonist, a CD47 antagonist, a PVRIG antagonist, a TDO antagonist, a VISTA antagonist, or a KIR antagonist.

In one embodiment, the checkpoint inhibitor is CDX-1127 (Celldex). In one embodiment, the checkpoint inhibitor is NKTR-214 (Nektar).

In one embodiment, the checkpoint inhibitor is an anti-LAG-3 antibody. Examples include relatlimab, BMS-986016, BI 754111, REGN-3767, LAG-525. Such antibodies are in clinical studies in combination with a PD-1 pathway inhibitor.

In one embodiment, the checkpoint inhibitor is a CTLA-4 antagonist. In certain embodiments, the CTLA-4 antagonist is an anti-CTLA-4 antibody or antigen binding fragment thereof. In some embodiments, the anti-CTLA-4 antibody is ipilimumab (YERVOY), tremelimumab (ticilimumab; CP-675,206), AGEN-1884, or ATOR-1015.

In one embodiment, the CTLA-4 antagonist is a soluble CTLA-4 polypeptide. In one embodiment, the soluble CTLA-4 polypeptide is abatacept (Orencia), belatacept (Nulojix), RG2077, or RG-1046. In another embodiment, the CTLA-4 antagonist is a cell based therapy. In some embodiments, the CTLA-4 antagonist is an anti-CTLA-4 mAb RNA/GITRL RNA-transfected autologous dendritic cell vaccine or an anti-CTLA-4 mAb RNA-transfected autologous dendritic cell vaccine.

In one embodiment, the checkpoint inhibitor is a KIR antagonist. In certain embodiments, the KIR antagonist is an anti-KIR antibody or antigen binding fragment thereof. In some embodiments, the anti-KIR antibody is lirilumab (1-7F9, BMS-986015, IPH 2101) or IPH4102.

In one embodiment, the checkpoint inhibitor is TIGIT antagonist. In one embodiment, the TIGIT antagonist is an anti-TIGIT antibody or antigen binding fragment thereof. In certain embodiments, the anti-TIGIT antibody is BMS-986207, AB 154, COM902 (CGEN-15137), or OMP-313M32.

In one embodiment, the checkpoint inhibitor is Tim-3 antagonist. In certain embodiments, the Tim-3 antagonist is an anti-Tim-3 antibody or antigen binding fragment thereof. In some embodiments, the anti-Tim-3 antibody is TSR-022 or LY3321367.

In one embodiment, the checkpoint inhibitor is an IDO1 antagonist. In another embodiment, the IDO1 antagonist is indoximod (LG8189; 1-methyl-D-TRP), epacadostat (INCB-024360, INCB-24360), KHK2455, PF-06840003, navoximod (RG6078, GDC-0919, LG919), BMS-986205 (F001287), or pyrrolidine-2,5-dione derivatives.

In one embodiment, the immune activating agent is a STING antagonist. In certain embodiments, the STING antagonist is 2′ or 3′-mono-fluoro substituted cyclic-di-nucleotides; 2′3′-di-fluoro substituted mixed linkage 2′,5′-3′,5′ cyclic-di-nucleotides; 2′-fluoro substituted, bis-3′,5′ cyclic-di-nucleotides; 2′,2″-diF-Rp,Rp,bis-3′,5′ cyclic-di-nucleotides; or fluorinated cyclic-di-nucleotides.

In one embodiment, the immune activating agent is CD20 antagonist. In some embodiments, the CD20 antagonist is an anti-CD20 antibody or antigen binding fragment thereof. In one embodiment, the anti-CD20 antibody is rituximab (RITUXAN; IDEC-102; IDEC-C2B8), ABP 798, ofatumumab, or obinutuzumab.

In one embodiment, the checkpoint inhibitor is CD80 antagonist. In certain embodiments, the CD80 antagonist is an anti-CD80 antibody or antigen binding fragment thereof. In one embodiment, the anti-CD80 antibody is galiximab or AV 1142742.

In one embodiment, the checkpoint inhibitor is a GARP antagonist. In some embodiments, the GARP antagonist is an anti-GARP antibody or antigen binding fragment thereof. In certain embodiments, the anti-GARP antibody is ARGX-115.

In one embodiment, the checkpoint inhibitor is a CD40 antagonist. In certain embodiments, the CD40 antagonist is an anti-CD40 antibody for antigen binding fragment thereof. In some embodiments, the anti-CD40 antibody is BMS3h-56, lucatumumab (HCD122 and CHIR-12.12), CHIR-5.9, or dacetuzumab (huS2C6, PRO 64553, RG 3636, SGN 14, SGN-40). In another embodiment, the CD40 antagonist is a soluble CD40 ligand (CD40-L). In one embodiment, the soluble CD40 ligand is a fusion polypeptide. In one embodiment, the soluble CD40 ligand is a CD40-L/FC2 or a monomelic CD40-L.

In one embodiment, the checkpoint inhibitor is an A2aR antagonist. In some embodiments, the A2aR antagonist is a small molecule. In certain embodiments, the A2aR antagonist is CPI-444, PBF-509, istradefylline (KW-6002), preladenant (SCH420814), tozadenant (SYN115), vipadenant (BIIB014), HTL-1071, ST1535, SCH412348, SCH442416, SCH58261, ZM241385, or AZD4635.

In one embodiment, the checkpoint inhibitor is a CEACAM1 antagonist.

In some embodiments, the CEACAM1 antagonist is an anti-CEACAM1 antibody or antigen binding fragment thereof. In one embodiment, the anti-CEACAM1 antibody is CM-24 (MK-6018).

In one embodiment, the immune activating agent is a CEA antagonist. In one embodiment, the CEA antagonist is an anti-CEA antibody or antigen binding fragment thereof. In certain embodiments, the anti-CEA antibody is cergutuzumab amunaleukin (RG7813, RO-6895882) or RG7802 (R06958688).

In one embodiment, the checkpoint inhibitor is a CD47 antagonist. In some embodiments, the CD47 antagonist is an anti-CD47 antibody or antigen binding fragment thereof. In certain embodiments, the anti-CD47 antibody is HuF9-G4, CC-90002, TTI-621, ALX148, NI-1701, NI-1801, SRF231, or Effi-DEM.

In one embodiment, the checkpoint inhibitor is a PVRIG antagonist. In certain embodiments, the PVRIG antagonist is an anti-PVRIG antibody or antigen binding fragment thereof. In one embodiment, the anti-PVRIG antibody is COM701 (CGEN-15029).

In one embodiment, the checkpoint inhibitor is a TDO antagonist. In one embodiment, the TDO antagonist is a 4-(indol-3-yl)-pyrazole derivative, a 3-indol substituted derivative, or a 3-(indol-3-yl)-pyridine derivative. In another embodiment, the immune checkpoint inhibitor is a dual IDO and TDO antagonist. In one embodiment, the dual IDO and TDO antagonist is a small molecule.

Some embodiments of this invention relate to administering to the patient a therapeutically effective amount of a cannabinoid or derivative thereof or a compound of Formula I-Ig, and a PD-1 pathway inhibitor.

Some embodiments of this invention relate to administering to the patient a therapeutically effective amount of a cannabinoid or derivative thereof or a compound of Formula I-Ig, and an immune checkpoint inhibitor.

In some embodiments of the invention, the immune activating agent for treating cancer is adoptive cell therapy, or a T-cell expressing a chimeric antigen receptor (CAR T-cell) or a T-cell expressing a modified T-cell receptor, wherein any of said cells recognizes a cancer cell. Examples of CAR T-cells include tisagenlecleucel (KYMRIAH), axicabtagene ciloleucel (YESCARTA), or JCARH125. Other therapies are Chimeric Antigen Receptor-Modified T Cells for CEA, Chimeric Antigen Receptor-Modified T Cells for Epcam (CARTEPC), Chimeric Antigen Receptor-Modified T Cells for CD22, Chimeric Antigen Receptor-Modified T Cells targeting CD123, Chimeric Antigen Receptor-Modified T Cells for CD30, Chimeric Antigen Receptor-Modified T Cells targeting CD19, Chimeric Antigen Receptor-Modified T Cells for EGFR806, Chimeric Antigen Receptor-Modified T Cells for HER2, Chimeric Antigen Receptor-Modified T Cells for P-BCMA-101 autologous T stem cell memory (Tscm) (P-BCMA-101), Chimeric Antigen Receptor-Modified T Cells for GD2, and Chimeric Antigen Receptor-Modified T Cells for VGFR2.

OX40 is a type of tumor necrosis factor (TNF) receptor, and is also called CD134. Anti-OX40 antibodies have shown clinical utility in treating cancer. In one embodiment, the checkpoint inhibitor is MEDI0562, MEDI6469 or MEDI6383.

In some embodiments of the invention, the immune activating agent is a bispecific antibody targeting both immune cells and tumor cells, like the bivalent bispecific T cell engagers (BITE) or tetravalent bispecific antibodies (TandAb). Several bispecific antibody formats have been developed. The BiTE (bispecific T cell engager) molecules have been very well characterized (reviewed in Nagorsen and Bauerle, Exp Cell Res 317, 1255-1260 (2011)). BiTEs are tandem scFv molecules wherein two scFv molecules are fused by a flexible linker. Further bispecific formats being evaluated for T cell engagement include diabodies (Holliger et al., Prot Eng 9, 299-305 (1996)) and derivatives thereof, such as tandem diabodies (Kipriyanov et al., J Mol Biol 293, 41-66 (1999)). A more recent development are the so-called DART (dual affinity retargeting) molecules, which are based on the diabody format but feature a C-terminal disulfide bridge for additional stabilization (Moore et al., Blood 117, 4542-51 (2011)).

In one aspect, the invention provides a method where the additional therapeutic agent is an oncolytic virus. Oncolytic viruses are viruses found in nature or modified, that reproduce selectively in cancer cells and specifically infect and kill tumor cells. It is a type of targeted therapy, also called oncolytic virotherapy, viral therapy, and virotherapy. Examples of oncolytic viruses include talimogene laherparepvec (T-VEC, or Imlygic®), Pexa-Vec (JX-594), TG6002, OBP-301, ADV/HSV-tk, LOAd703, GL-ONC1, and CG0070.

Some embodiments of this invention relate to administering to the patient a therapeutically effective amount of a cannabinoid or derivative thereof or a compound of Formula I-Ig, to upregulate MHC-I. Such methods typically include administering to the subject an effective amount of the compound or the pharmaceutically acceptable salt thereof, the tautomer thereof, the pharmaceutically acceptable salt of the tautomer, the stereoisomer of any of the foregoing, or the mixture thereof according to any one of the embodiments or a pharmaceutical composition of any of the embodiments. In such embodiments, the MHC-I is increased in the subject after administration.

Some embodiments of this invention relate to a method for the prevention or treatment of cancer in a subject, the method comprising administering to said subject a cannabinoid or derivative thereof or a compound of Formula I-Ig. Such method typically includes administering to the subject an effective amount of the compound or the pharmaceutically acceptable salt thereof, the tautomer thereof, the pharmaceutically acceptable salt of the tautomer, the stereoisomer of any of the foregoing, or the mixture thereof according to any one of the embodiments or a pharmaceutical composition of any of the embodiments.

The invention also relates to the use of the compounds of the present invention in adjuvant or neoadjuvant chemotherapy, with or without radiation, for the treatment of neoplasia. “Adjuvant chemotherapy” is defined as the continued treatment after either intensive cycles of chemotherapy and/or radiation, or alternatively after surgery to remove tumors, Alternatively the term describes the use of drugs as additional treatment for patients with cancers that are thought to have spread outside their original sites. Neo-adjuvant therapy is defined as intensive cycles of chemotherapy and/or radiation given to reduce the size of tumor before a definitive surgery.

The invention also relates to the use of the compounds of the present invention in second or greater line treatment. The invention also relates to the use of the compounds of the present invention for the treatment of patients with recurrent or metastatic disease with disease progression on or after at least one prior chemotherapy regimen.

The invention also relates to the use of the compounds of the present invention following at least one prior chemotherapy regimen.

The invention also relates to the use of the compounds of the present invention in treatment of refractory cancer or relapse following therapy.

The invention also relates to the use of the compounds of the present invention in maintenance treatment.

Some embodiments of this invention relate to a method for the prevention or treatment of cancer in a subject, wherein said cancer is not characterized by dysregulation of the IL-10 and/or GM-CSF pathways in cancerous cells.

Since one aspect of the present invention contemplates the treatment of the disease/conditions with a combination of pharmaceutically active compounds that may be administered separately, the invention further relates to combining separate pharmaceutical compositions in kit form. The kit comprises two separate pharmaceutical compositions: the compound of the present invention, and a second pharmaceutical compound. The kit comprises a container for containing the separate compositions such as a divided bottle or a divided foil packet. Additional examples of containers include syringes, boxes and bags. Typically, the kit comprises directions for the use of the separate components. The kit form is particularly advantageous when the separate components are preferably administered in different dosage forms (e.g., oral and parenteral), are administered at different dosage intervals, or when titration of the individual components of the combination is desired by the prescribing physician or veterinarian.

Some embodiments of this invention relate to a method of identifying a patient with a malignant tumor who is likely to respond to a therapy with a cannabinoid compound or derivative, the method comprising:
(a) determining the level of MHC-I expression in a tumor sample; and
(b) identifying the patient who is likely to respond to treatment if the tumor is MHC-I deficient.

Some embodiments of this invention relate to a method of selecting a patient with a malignant tumor for a therapy with a cannabinoid compound or derivative, the method comprising:
(a) determining the level of MHC-I expression in a tumor sample; and
(b) selecting the patient for a therapy with a cannabinoid compound or derivative if the tumor is an MHC-I deficient tumor.

Some embodiments of this invention relate to method of treating a patient having cancer with a cannabinoid or cannabinoid derivative, the method comprising:
(a) determining the level of MHC-I expression in a tumor sample;
(b) measure MHC-I expression in the tumor sample after test treatment ex vivo with a cannabinoid or cannabinoid derivative;
(c) administer a cannabinoid or cannabinoid derivative to the patient if the MHC-I level in the tumor sample increases after said test treatment.

MHC-I expression can be measured by methods known in the art, such as with flow cytometry, fluorescence activated cell sorting [FACS], immunocytochemistry (ICC), Western blotting, antibody array and immunohistochemistry (IHC). MHC-I message can be measured by Northern blot, quantitative PCR, dot blot, and RNA sequencing.

A tumor is defined as MHC-I deficient or down-regulated if the assay as described herein provides a result that is less than the related primary cell line. Methods of treating patients selected in such a manner are contemplated. The invention specifically contemplates treating patients with cancer cells that are MHC-I deficient or MHC-I down-regulated.

Also provided are pharmaceutical compositions that include at least one pharmaceutically acceptable excipient, carrier or diluent and the compound or the pharmaceutically acceptable salt thereof, the tautomer thereof, the pharmaceutically acceptable salt of the tautomer, the stereoisomer of any of the foregoing, or the mixture thereof according to any one of the embodiments.

In one aspect, the invention provides a method for increasing major histocompatibility complex class I (MHC-I) surface expression in a cell, comprising contacting the cell with a compound of Formula I, or a pharmaceutically acceptable salt thereof;

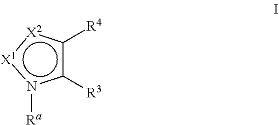

Wherein X¹ is —CR⁵—, nitrogen or —NR⁵—;
Wherein X² is —CR²—, nitrogen or —NR⁵—; provided X² is not nitrogen or —NR⁵— if X¹ is nitrogen or —NR⁵—;
Wherein R^{a} is absent, H, or R¹;
Wherein R¹ is selected from alkyl, haloalkyl, cyanoalkyl, alkenyl, heterocyclyl, cycloalkylalkyl, heterocyclylalkyl, arylalkyl and aryl;
Wherein R² is selected from H, aryl, aminocarbonylalkylaminocarbonyl, alkoxycarbonylalkylaminocarbonyl, aminocarbonyl(arylalkyl)aminocarbonyl, alkenylcarbonyl, arylcarbonyl, cycloalkylcarbonyl, arylalkylcarbonyl, heterocyclylalkylcarbonyl, heterocyclylcarbonyl, alkoxycarbonylalkyloxycarbonyl, cycloalkyloxycarbonyl, aryloxycarbonyl, heterocyclyloxycarbonyl, arylaminocarbonyl, arylalkylaminocarbonyl, heterocyclylaminocarbonyl, cycloalkylaminocarbonyl, optionally substituted arylalkyl and heterocyclylcarbonylalkyl;
Wherein R³ is H or aryl;
Wherein R⁴ is H or amino;
Wherein R⁴ and R³ together form a 6-membered aryl or heteroaryl ring, wherein the ring is optionally substituted with one or more substituents selected from halo, nitro, C₁₋₆-alkoxy, alkylaminocarbonyl, or optionally substituted C₆₋₁₀ aryl; and
Wherein R⁵ is selected from H, alkyl, arylcarbonyl, aminocarbonylalkylaminocarbonyl, arylalkyl, haloalkyl, cycloalkylalkyl, and cyanoalkyl; or a pharmaceutically acceptable salt thereof.

In one aspect, the invention provides a method for increasing major histocompatibility complex class I (MHC-I) surface expression in a cell, comprising contacting the cell with a compound of Formula Ia

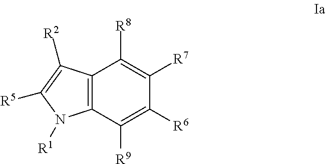

Wherein R¹ is selected from alkyl, haloalkyl, cyanoalkyl, alkenyl, cycloalkylalkyl, heterocyclyl, heterocyclylalkyl, optionally substituted arylalkyl and optionally substituted aryl; R² is aminocarbonylalkylaminocarbonyl, alkoxycarbonylalkylaminocarbonyl, aminocarbonyl(optionally substituted arylalkyl)aminocarbonyl, alkenylcarbonyl, optionally substituted arylcarbonyl, optionally substituted arylalkylcarbonyl, heterocyclylalkylcarbonyl, heterocyclylcarbonyl, heterocyclyloxycarbonyl, optionally substituted arylaminocarbonyl, heterocyclylaminocarbonyl, cycloalkylaminocarbonyl, cycloalkylcarbonyl, optionally substituted arylalkyl, and heterocyclylcarbonylalkyl; R⁵ is H, alkyl or arylcarbonyl; R⁶ is H, halo, nitro, C₁₋₆-alkoxy or optionally substituted C₆₋₁₀ aryl; R⁷ is H; R⁸ is H; and R⁹ is H; or pharmaceutically acceptable salt thereof.

In some embodiments, the method comprises compounds of Formula Ia wherein R¹ is selected from C₁₋₈-alkyl, C₁₋₆-haloalkyl, C₁₋₆-cyanoalkyl, C₂₋₆-alkenyl, C₃₋₈-cycloalkyl-C₁₋₆-alkyl, 3-8 membered heterocyclyl, 3-8 membered heterocyclyl-C₁₋₆ alkyl, optionally substituted phenyl-C₁₋₆-alkyl and optionally substituted phenyl; R² is aminocarbonyl(C₁₋₆-alkyl)aminocarbonyl, C₁₋₆-alkoxycarbonyl(C₁₋₆-alkyl)aminocarbonyl, aminocarbonyl(optionally substituted phenyl-C₁₋₆-alkyl)aminocarbonyl, C₂₋₈-alkenylcarbonyl, optionally substituted C₆₋₁₀-arylcarbonyl, optionally substituted phenyl-C₁₋₆-alkylcarbonyl, 3-10 membered heterocyclyl-C₁₋₆-alkylcarbonyl, 3-10 membered heterocyclylcarbonyl, 3-10 membered heterocyclyloxycarbonyl, optionally substituted C₆₋₁₀-arylaminocarbonyl, 3-10 membered heterocyclylaminocarbonyl, C₃₋₈-cycloalkylaminocarbonyl, C₃₋₈-cycloalkylcarbonyl, optionally substituted C₆₋₁₀-aryl-C₁₋₆-alkyl, and 3-10 membered heterocyclylcarbonyl-C₁₋₆-alkyl; R⁵ is H, C₁₋₆-alkyl or arylcarbonyl; and R⁶ is H, halo, nitro, C₁₋₆-alkoxy or optionally substituted C₆₋₁₀ aryl; or pharmaceutically acceptable salt thereof.

In some embodiments, the method comprises compounds of Formula Ia wherein R¹ is selected from propyl, butyl, pentyl, 1-methylpropyl, 2-methylpropyl, 1,1-dimethylpropyl, 1,2-dimethylpropyl, 2,2-dimethylpropyl, tert-butyl, 1-methylbutyl, 2-methylbutyl, 3-methylbutyl, 1-ethylpropyl, hexyl, 1-methylhexyl, 5-methylhexyl, heptyl, 3-chlorobutyl, 2-chloropentyl, 3-chloropentyl, 4-chloropentyl, 5-chloropentyl, 2-fluoropentyl, 3-fluoropentyl, 4-fluoropentyl, 5-fluoropentyl, 5-bromopentyl, 2-chlorohexyl, 3-chlorohexyl, 4-chlorohexyl, 5-chlorohexyl, 6-chlorohexyl, 4-cyanobutyl, penten-4-yl, cyclohexylmethyl, cyclohexylethyl, 1-methyl-azepan-3-yl, 4-morpholinylmethyl, 4-morpholinylethyl, 2-oxiranylpropyl, 1-methylpiperidin-2-ylmethyl, 4-fluorophenylmethyl and 4-chlorophenylcarbonyl; R² is aminocarbonyl-(2,2-dimethylpropyl)aminocarbonyl, aminocarbonyl-(2-methylpropyl)aminocarbonyl, methoxycarbonyl-(2,2-dimethylethyl)aminocarbonyl, methoxycarbonyl-(2,2-dimethylpropyl)aminocarbonyl, methoxycarbonyl-(2-methylpropyl)aminocarbonyl, aminocarbonyl(phenylethyl)aminocarbonyl, 2,3,3-trimethyl-1-but-1-enylcarbonyl, 4-hydroxyphenylcarbonyl, 2-iodo-5-nitophenylcarbonyl, 2-iodophenylcarbonyl, 3-iodophenylcarbonyl, 4-iodophenylcarbonyl, 2-methoxyphenylcarbonyl, 3-methoxyphenylcarbonyl, 4-methoxyphenylcarbonyl, 1-naphthylcarbonyl, 2-naphthylcarbonyl, 1-methoxy-4-naphthylcarbonyl, 2-methoxy-1-naphthylcarbonyl, 3-methoxy-1-naphthylcarbonyl, 5-methoxy-1-naphthylcarbonyl, 6-methoxy-1-naphthylcarbonyl, 7-methoxy-1-naphthylcarbonyl, 1-methyl-4-naphthylcarbonyl, 2-methyl-1-naphthylcarbonyl, 3-methyl-1-naphthylcarbonyl, 4-methyl-1-naphthylcarbonyl, 5-methyl-1-naphthylcarbonyl, 6-methyl-1-naphthylcarbonyl, 7-methyl-1-naphthylcarbonyl, 8-methyl-1-naphthylcarbonyl, 2-ethyl-1-naphthylcarbonyl, 3-ethyl-1-naphthylcarbonyl, 5-ethyl-1-naphthylcarbonyl, 6-ethyl-1-naphthylcarbonyl, 7-ethyl-1-naphthylcarbonyl, 8-ethyl-1-naphthylcarbonyl, 1-ethyl-4-naphthylcarbonyl, 1-propyl-4-naphthylcarbonyl, 2-propyl-1-naphthylcarbonyl, 1-fluoro-4-naphthylcarbonyl, 1-bromo-4-naphthylcarbonyl, 8-bromo-1-naphthylcarbonyl, 1-chloro-4-naphthylcarbonyl, 2-chloro-1-naphthylcarbonyl, 3-chloro-1-naphthylcarbonyl, 5-chloro-1-naphthylcarbonyl, 6-chloro-1-naphthylcarbonyl, 7-chloro-1-naphthylcarbonyl, 8-chloro-1-naphthylcarbonyl, 4-hydroxyethylnaphth-1-ylcarbonyl, phenylmethylcarbonyl, 2-methylphenylmethylcarbonyl, 3-methylphenylmethylcarbonyl, 4-methylphenylmethylcarbonyl, 2-methoxyphenylmethylcarbonyl, 4-methoxyphenylmethylcarbonyl, 3-methoxyphenylmethylcarbonyl, 2-chlorophenylmethylcarbonyl, 3-chlorophenylmethylcarbonyl, 4-chlorophenylmethylcarbonyl, 2-bromophenylmethylcarbonyl, morphlinylmethylcarbonyl, 2,2,4-trimethyl-3-bicyclo[2.2.1]heptylaminocarbonyl, 1-pyrollidinylcarbonyl, 4-benzylpiperazin-1-ylcarbonyl, 3-pyridylcarbonyl, quinolin-3-yloxycarbonyl, quinolin-4-yloxycarbonyl, quinolin-5-yloxycarbonyl, quinolin-6-yloxycarbonyl, quinolin-7-yloxycarbonyl, quinolin-8-yloxycarbonyl, 4-isoquinolinyloxycarbonyl, 5-isoquinolinyloxycarbonyl, 6-isoquinolinyloxycarbonyl, 7-isoquinolinyloxycarbonyl, 8-isoquinolinyloxycarbonyl, 2,2,3,3-tetramethylcyclopropylcarbonyl, adamantylcarbonyl, 4-methylpiperazin-1-ylcarbonyl, 3,4-dimethylpiperazin-1-ylcarbonyl, phenylaminocarbonyl, phenylmethylaminocarbonyl, phenyl-(1,1-dimethylmethyl)aminocarbonyl, 1-naphthylaminocarbonyl, adamantylaminocarbonyl, cyclopropylaminocarbonyl, 2,2,4-trimethyl-3-bicyclo[2.2.1]heptanylaminocarbonyl, naphthylmethyl, and morpholinylcarbonylmethyl; R⁵ is H, methyl or ethyl; and R⁶ is H, bromo, nitro, Iodo, methoxy or 4-methylnaphtyl; or pharmaceutically acceptable salt thereof.

In some embodiments, the method comprises compounds of Formula Ia wherein R¹ is selected from butyl, pentyl, 2-methylpropyl, 2,2-dimethylpropyl, 2-methylbutyl, 1-ethylpropyl, 3-chloropentyl, 3-fluoropentyl, 5-fluoropentyl, 5-bromopentyl, cyclohexylmethyl, cyclohexylethyl, 1-methyl-azepan-3-yl, 4-morpholinylethyl, 4-fluorophenylmethyl and 1-methylpiperidin-2-ylmethyl; R² is aminocarbonyl-(2,2-dimethylpropyl)aminocarbonyl, methoxycarbonyl-(2,2-dimethylpropyl)aminocarbonyl, 2-iodo-5-nitophenylcarbonyl, 2-methoxyphenylcarbonyl, 2-iodophenylcarbonyl, 1-naphthylcarbonyl, 2-naphthylcarbonyl, 1-methoxy-4-naphthylcarbonyl, 2-methyl-1-naphthylcarbonyl, 8-chloro-1-naphthylcarbonyl, 4-methoxyphenylmethylcarbonyl, 3-methoxyphenylmethylcarbonyl, 2,2,4-trimethyl-3-bicyclo[2.2.1]heptylaminocarbonyl, quinolin-4-yloxycarbonyl, quinolin-6-yloxycarbonyl, 4-isoquinolinyloxycarbonyl, 7-isoquinolinyloxycarbonyl, quinolin-6-yloxycarbonyl, quinolin-7-yloxycarbonyl, quinolin-8-yloxycarbonyl, 2,2,3,3-tetramethylcyclopropylcarbonyl, and 8-isoquinolinyloxycarbonyl, R⁵ is H; and R⁶ is H or methoxy; or pharmaceutically acceptable salt thereof.

In one aspect, the invention provides a method for increasing major histocompatibility complex class I (MHC-I) surface expression in a cell, comprising contacting the cell with a compound of Formula Ib

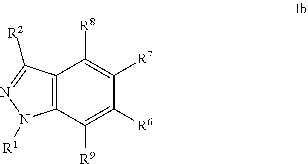

wherein R¹ is selected from alkyl, haloalkyl, cyanoalkyl, cycloalkylalkyl, heterocyclylalkyl, arylalkyl and aryl; R² is aminocarbonyl(alkyl)aminocarbonyl, alkoxycarbonyl(alkyl)aminocarbonyl, aminocarbonyl(arylalkyl)aminocarbonyl, arylcarbonyl, heterocyclylcarbonyl, aralkylcarbonyl, aryloxycarbonyl, alkoxycarbonyl(alkyl)oxycarbonyl, cycloalkyloxycarbonyl, heterocyclyloxycarbonyl, arylalkylaminocarbonyl, heterocyclylaminocarbonyl, and cycloalkylcarbonyl; R⁶ is H; R⁷ is H; R is H; and R⁹ is H; or pharmaceutically acceptable salt thereof.

In some embodiments, the method comprises compounds of Formula Ib wherein R¹ is selected from C₁₋₈-alkyl, C₁₋₆-haloalkyl, C₁₋₆-cyanoalkyl, C₃₋₈-cycloalkyl-C₁₋₆-alkyl, 3-8 membered heterocyclyl-C₁₋₆ alkyl, optionally substituted phenyl-C₁₋₆-alkyl and optionally substituted phenyl; and R² is aminocarbonyl(C₁₋₆-alkyl)aminocarbonyl, C₁₋₆-alkoxycarbonyl(C₁₋₆-alkyl)aminocarbonyl, aminocarbonyl(optionally substituted phenyl-C₁₋₆-alkyl)aminocarbonyl, optionally substituted C₆₋₁₀-arylcarbonyl, 3-10 membered heterocyclylcarbonyl, optionally substituted phenyl-C₁₋₆-alkylcarbonyl, C₁₋₆-alkoxycarbonyl(C₁₋₆-alkyl)oxycarbonyl, optionally substituted C₆₋₁₀-aryloxycarbonyl, C₃₋₈-cycloalkyloxycarbonyl, 3-10 membered heterocyclyloxycarbonyl, optionally substituted phenyl-C₁₋₆-alkylaminocarbonyl, 3-10 membered heterocyclylaminocarbonyl, and C₃₋₈-cycloalkylcarbonyl; or pharmaceutically acceptable salt thereof.

In some embodiments, the method comprises compounds of Formula Ib wherein R¹ is selected from pentyl, 2-fluoropentyl, 3-fluoropentyl, 4-fluoropentyl, 5-fluoropentyl, 5-bromopentyl, 5-chloropentyl, 4-cyanobutyl, cyclohexylmethyl, 4-morpholinylmethyl, 2-fluorophenyl, benzyl, 3-fluorobenzyl, 2-fluorobenzyl, and 4-fluorophenylmethyl; and R² is aminocarbonyl-(butyl)aminocarbonyl, aminocarbonyl-(pentyl)aminocarbonyl, aminocarbonyl-(2,2-dimethylpropyl)aminocarbonyl, aminocarbonyl-(1-methylpropyl)aminocarbonyl, aminocarbonyl-(2-methylpropyl)aminocarbonyl, aminocarbonyl-(2-methylbutyl)aminocarbonyl, aminocarbonyl-(3-methylbutyl)aminocarbonyl, aminocarbonyl-(1,1-dimethylpropyl)aminocarbonyl, methoxycarbonyl-(2,2-dimethylethyl)aminocarbonyl, methoxycarbonyl-(2,2-dimethylpropyl)aminocarbonyl, methoxycarbonyl-(1,1-dimethylpropyl)aminocarbonyl, ethoxycarbonyl-(2,2-dimethylethyl)aminocarbonyl, aminocarbonyl(phenylethyl)aminocarbonyl, 1-naphthylcarbonyl, 1-pyrollidinylcarbonyl, phenyl-(1,1-dimethyl)methylcarbonyl, methoxycarbonyl-(2,2-dimethylpropyl)oxycarbonyl, adamantyloxycarbonyl, 1-naphthyloxycarbonyl, 2-naphthyloxycarbonyl, quinolin-8-yloxycarbonyl, 2,2,3,3-tetramethylcyclopropylcarbonyl, 1-naphthylaminocarbonyl, adamantylaminocarbonyl, phenyl-(1,1-dimethyl)methylaminocarbonyl, and 8-quinolinylaminocarbonyl; or pharmaceutically acceptable salt thereof.

In some embodiments, the method comprises compounds of Formula Ib wherein R¹ is selected from pentyl, cyclohexylmethyl and 4-fluorophenylmethyl; R² is aminocarbonyl-(pentyl)aminocarbonyl, aminocarbonyl-(2-methylbutyl)aminocarbonyl, aminocarbonyl-(3-methylbutyl)aminocarbonyl, methoxycarbonyl-(2,2-dimethylethyl)aminocarbonyl, methoxycarbonyl-(1,1-dimethylpropyl)aminocarbonyl, methoxycarbonyl-(2,2-dimethylpropyl)oxycarbonyl, aminocarbonyl(phenylethyl)aminocarbonyl and ethoxycarbonyl-(2,2-dimethylethyl)aminocarbonyl; or pharmaceutically acceptable salt thereof.

In one aspect, the invention provides a method for increasing major histocompatibility complex class I (MHC-I) surface expression in a cell, comprising contacting the cell with a compound of Formula Ic

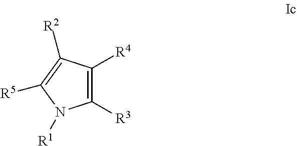

Wherein R¹ is alkyl or arylalkyl; R² is H or arylcarbonyl; R³ is H or aryl; R⁴ is H or amino; and R⁵ is H, C₁₋₆-alkyl or arylcarbonyl; or pharmaceutically acceptable salt thereof.

In some embodiments, the method comprises compounds of Formula Ic wherein R¹ is C₁₋₆-alkyl or aryl-C₁₋₆-alkyl; R² is H or C₆₋₁₀-arylcarbonyl; R³ is H or optionally substituted C₆₋₁₀-aryl; R⁴ is H or amino; and R is H, C₁₋₆-alkyl or C₆₋₁₀-arylcarbonyl; or pharmaceutically acceptable salt thereof.

In some embodiments, the method comprises compounds of Formula Ic wherein R¹ is pentyl, hexyl, heptyl or 4-chlorophenylmethyl; R² is H, phenylcarbonyl or naphthylcarbonyl; R³ is H, phenyl, 2-methylphenyl, 2-fluorophenyl, 2-chlorophenyl, 3-fluorophenyl, or naphthyl; R⁴ is H or amino; and R⁵ is H, methyl or naphthylcarbonyl; or pharmaceutically acceptable salt thereof.

In some embodiments, the method comprises compounds of Formula Ic wherein R² is arylcarbonyl when R⁵ is H or alkyl; or pharmaceutically acceptable salt thereof.

In one aspect, the invention provides a method for increasing major histocompatibility complex class I (MHC-I) surface expression in a cell, comprising contacting the cell with a compound of Formula Id

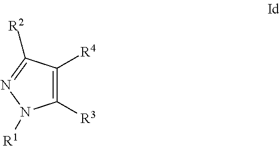

Wherein R¹ is haloalkyl or cycloalkylalkyl; R² is aminocarbonyl(alkyl)aminocarbonyl or optionally substituted aryl; R³ is optionally substituted aryl; and R⁴ is H; or pharmaceutically acceptable salt thereof.

In some embodiments, the method comprises compounds of Formula Id wherein R¹ is C₁₋₆-haloalkyl or C₃₋₆-cycloalkyl-C₁₋₆-alkyl; R² is aminocarbonyl-(C₁₋₆-alkyl)aminocarbonyl or optionally substituted phenyl; R³ is optionally substituted phenyl or aminocarbonyl-(C₁₋₆-alkyl)aminocarbonyl; and R⁴ is H; or pharmaceutically acceptable salt thereof.

In some embodiments, the method comprises compounds of Formula Id wherein R¹ is 5-fluoropentyl or cyclohexylmethyl; R² is aminocarbonyl-(1,1-dimethylethyl)aminocarbonyl or 4-fluorophenyl; R³ is aminocarbonyl-(2,2-dimethylpropyl)aminocarbonyl or aminocarbonyl-(1,1-dimethylethyl)aminocarbonyl or 4-fluorophenyl; and R⁴ is H; or pharmaceutically acceptable salt thereof.

In some embodiments, the method comprises compounds of Formula Id wherein R² is aminocarbonyl-(1,1-dimethylethyl)aminocarbonyl when R³ is 4-fluorophenyl; or pharmaceutically acceptable salt thereof.

In one aspect, the invention provides a method for increasing major histocompatibility complex class I (MHC-I) surface expression in a cell, comprising contacting the cell with a compound of Formula Ie

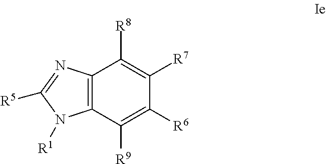

Wherein R¹ is alkyl, haloalkyl or cycloalkylalkyl; R⁵ is arylcarbonyl, aminocarbonylalkylaminocarbonyl or optionally substituted arylalkyl; R⁶ is H; R⁷ is H; R⁸ is H; and R is H or alkylaminocarbonyl; or pharmaceutically acceptable salt thereof.

In some embodiments, the method comprises compounds of Formula Ie wherein R¹ is C₁₋₆-alkyl, C₁₋₆-haloalkyl or cyclohexyl-C₁₋₆-alkyl; R⁵ is C₆₋₁₀-aryl-C₁₋₆-alkylcarbonyl, aminocarbonyl-C₁₋₆-alkylaminocarbonyl or substituted phenyl-C₁₋₆-alkyl; and R⁶ is H or C₁₋₆-alkylaminocarbonyl; or pharmaceutically acceptable salt thereof.

In some embodiments, the method comprises compounds of Formula Ie wherein R¹ is pentyl, 5-fluoropentyl, 2-hydroxy-2methylbutyl or cyclohexylmethyl; R⁵ is naphthylcarbonyl, aminocarbonyl-(1,1-di-methylpropyl)aminocarbonyl or 4-ethoxyphenylmethyl; and R⁶ is H or diethylaminocarbonyl; or pharmaceutically acceptable salt thereof.

In some embodiments, the method comprises compounds of Formula Ie wherein R⁵ is optionally substituted arylalkyl when R⁶ is alkylaminocarbonyl; or pharmaceutically acceptable salt thereof.

In one aspect, the invention provides a method for increasing major histocompatibility complex class I (MHC-I) surface expression in a cell, comprising contacting the cell with a compound of Formula If

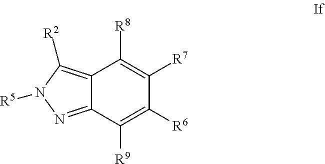

Wherein R² is arylaminocarbonyl, aminocarbonylalkylaminocarbonyl or arylalkylcarbonyl; R⁵ is haloalkyl, cycloalkylalkyl, alkyl or cyanoalkyl; R⁶ is H; R⁷ is H; R⁸ is H; and R⁹ is H; or pharmaceutically acceptable salt thereof.

In some embodiments, the method comprises compounds of Formula If wherein R² is arylaminocarbonyl, aminocarbonyl-C₁₋₆-alkylaminocarbonyl or aryl-C₁₋₆-alkylcarbonyl; and R⁵ is C₁₋₈-haloalkyl, cyclohexyl-C₁₋₆-alkyl, C₁₋₆-alkyl or cyano-C₁₋₆-alkyl; or pharmaceutically acceptable salt thereof.

In some embodiments, the method comprises compounds of Formula If wherein R² is naphthylaminocarbonyl, aminocarbonyl-(2-methylbutyl)aminocarbonyl or 1,1-dimethylphenylmethylcarbonyl; and R⁵ is 5-fluoropentyl, cyclohexylmethyl, pentyl or 4-cyanobutyl; or pharmaceutically acceptable salt thereof.

In one aspect, the invention provides a method for increasing major histocompatibility complex class I (MHC-I) surface expression in a cell, comprising contacting the cell with a compound of Formula Ig

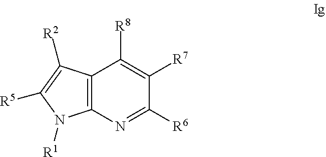

Wherein R¹ is haloalkyl; R² is arylalkylcarbonyl; R⁵ is H; R⁶ is H; R⁷ is H; and R⁸ is H; or pharmaceutically acceptable salt thereof.

In some embodiments, the method comprises compounds of Formula Ig wherein R¹ is C₁₋₆-haloalkyl; and R² is aryl-C₁₋₆-alkylcarbonyl; or pharmaceutically acceptable salt thereof.

In some embodiments, the method comprises compounds of Formula Ig wherein R¹ is 5-fluoropentyl; and R² is 1,1-dimethylphenylmethylcarbonyl; or pharmaceutically acceptable salt thereof.

In one aspect, the invention provides a method wherein the cannabinoid compound or derivative thereof is a compound of table 1.

TABLE 1
Cannabinoid Compounds and Derivatives.

| Example | Synonym | Compound |
|---|---|---|
| 1 | CBG, cannabigerol | 2-[(2E)-3,7-dimethyl-2,6-octadien-1-yl]-5-pentyl- |
| | | 1,3-benzenediol |
| 2 | CBD, cannabidiol | 2-[1R-3-methyl-6R-(1-methylethenyl)-2- |
| | | cyclohexen-1-yl]-5-pentyl-1,3-benzenediol |
| 3 | CBC, cannabichromene | 2-methyl-2-(4-methyl-3-penten-1-yl)-7-pentyl-2H- |
| | | 1-benzopyran-5-ol |
| 4 | CBN, cannabinol | 6,6,9-trimethyl-3-pentyl-6H-dibenzo[b,d]pyran-1- |
| | | ol |
| 5 | THV, | 6aR,7,8,10aR-tetrahydro-6,6,9-trimethyl-3-propyl- |
| | Tetrahydrocannabivarin | 6H-dibenzo[b,d]pyran-1-ol |
| c | AM1220 azepane | (1-((1-methylazepan-3-yl)methyl)-1H-indol-3- |
| | isomer | yl)(naphthalen-1-yl)methanone |
| 7 | AM1241 | (2-iodo-5-nitrophenyl)-(1-(1-methylpiperidin-2- |
| | | ylmethyl)-1H-indol-3-yl)methanone |
| 8 | EMB-FUBINACA | ethyl (1-(4-fluorobenzyl)-1H-indazole-3- |
| | | carbonyl)-1-valinate |
| 9 | BB-22 4-HO-quinoline | quinolin-4-yl 1-(cyclohexylmethyl)-1H-indole-3- |
| | isomer | carboxylate |
| 10 | PB-22 4-HO-quinoline | quinolin-4-yl 1-pentyl-1H-indole-3-carboxylate |
| | isomer | |
| 11 | ADB-PINACA | (S)-N-(1-amino-1-oxohexan-2-yl)-1-pentyl-1H- |
| | isomer 3 | indazole-3-carboxamide |
| 12 | 5-fluoro PB-22 | 7-hydroxyquinoline isomer quinolin-7-yl 1-(5- |
| | | fluoropentyl)-1H-indole-3-carboxylate |
| 13 | JWH 200 2′-naphthyl | [1-[2-(4-morpholinyl)ethyl]-1H-indol-3-yl]-2- |
| | isomer | naphthalenyl-methanone |
| 14 | MA-CHMINACA | N-[[1-(cyclohexylmethyl)-1H-indazol-3- |
| | | yl]carbonyl]-L-valine, methyl ester |
| 15 | MMB-FUBINACA | N-[[1-[(4-fluorophenyl)methyl]-1H-indazol-3- |
| | | yl]carbonyl]-L-valine, methyl ester |
| 16 | 5-fluoro PB-22 8-HO- | isoquinolin-8-yl 1-(5-fluoropentyl)-1H-indole-3- |
| | isoquinoline isomer | carboxylate |
| 17 | PB-22 8-HO- | isoquinolin-8-yl 1-pentyl-1H-indole-3-carboxylate |
| | isoquinoline isomer | |
| 18 | PB-22 7-HO-quinoline | quinolin-7-yl 1-pentyl-1H-indole-3-carboxylate |
| | isomer | |
| 19 | 5-fluoro PB-22 4-HO- | isoquinolin-4-yl 1-(5-fluoropentyl)-1H-indole-3- |
| | isoquinoline isomer | carboxylate |
| 20 | RCS-4 2-methoxy | (2-methoxyphenyl)(1-pentyl-1H-indol-3-yl)- |
| | isomer | methanone |
| 21 | JWH 198 | (4-methoxy-1-naphthalenyl)[1-[2-(4- |
| | | morpholinyl)ethyl]-1H-indol-3-yl]-methanone |
| 22 | JWH 073 2-CH₃- | (1-butyl-1H-indol-3-yl)(2-methylnaphthalen-1- |
| | naphthyl analog | yl)methanone |
| 23 | (R)-AM1241 | (2-iodo-5-nitrophenyl)[1-[[(2R)-1-methyl-2- |
| | | piperidinyl]methyl]-1H-indol-3-yl]methanone |
| 24 | AM2233 azepane | (2-iodophenyl)(1-(1-methylazepan-3-yl)-1H-indol- |
| | isomer | 3-yl)methanone |
| 25 | JWH 122 2-CH3- | (2-methylnaphthalen-1-yl)(1-pentyl-1H-indol-3- |
| | naphthyl isomer | yl)methanone |
| 26 | ADB-PINACA | N-((2S,3S)-1-amino-3-methyl-1-oxopentan-2-yl)- |
| | isomer 2 | 1-pentyl-1H-indazole-3-carboxamide |
| 27 | URB447 | [4-amino-1-[(4-chlorophenyl)methyl]-2-methyl-5- |
| | | phenyl-1H-pyrrol-3-yl]phenyl-methanone |
| 28 | JWH 302 | 2-(3-methoxyphenyl)-1-(1-pentyl-1H-indol-3-y1)- |
| | | ethanone |
| 29 | APP-CHMINACA | N-[(1S)-2-amino-2-oxo-1-(phenylmethyl)ethyl]-1- |
| | | (cyclohexylmethyl)-1H-Indazole-3-carboxamide |
| 30 | 5-fluoro PB-22 6-HO- | quinolin-6-yl 1-(5-fluoropentyl)-1H-indole-3- |
| | quinoline isomer | carboxylate |
| 31 | MDMB-CHMICA | N-[[1-(cyclohexylmethyl)-1H-indol-3- |
| | | yl]carbonyl]-3-methyl-1-valine, methyl ester |
| 32 | AM1220 | [1-[(1-methyl-2-piperidinyl)methyl]-1H-indol-3- |
| | | yl]-1-naphthalenyl-methanone |
| 33 | MAB-CHMINACA | N-[1-(aminocarbonyl)-2,2-dimethylpropyl]-1- |
| | | (cyclohexylmethyl)-1H-indazole-3-carboxamide |
| 34 | JWH 018 N-(3- | N-(3-Methylbutyl)-3-(1-naphthoyl)-indole |
| | methylbutyl) isomer | |
| 35 | JWH 073 | (1-butyl-1H-indol-3-yl)-1-naphthalenyl- |
| | | methanone |
| 36 | AM679 | (2-iodophenyl)(1-pentyl-1H-indol-3-yl)- |
| | | methanone |
| 37 | MDMB-CHMINACA | N-[[1-(cyclohexylmethyl)-1H-indazol-3- |
| | | yl]carbonyl]-3-methyl-1-valine, methyl ester |
| 32 | MO-CHMINACA | 1-methoxy-3,3-dimethyl-1-oxobutan-2-yl 1- |
| | | (cyclohexylmethyl)-1H-indazole-3-carboxylate |
| 33 | ADB-PINACA | (S)-N-(1-amino-4-methyl-1-oxopentan-2-yl)-1- |
| | isomer 4 | pentyl-1H-indazole-3-carboxamide |
| 34 | AM2233 | (2-iodophenyl)[1-[(1-methyl-2- |
| | | piperidinyl)methyl]-1H-indol-3-yl]-methanone |
| 35 | JWH 0180 N-(5- | (1-(5-bromopentyl)-1H-indol-3-yl)(naphthalen-1- |
| | bromopentyl) analog | yl)methanone |
| 36 | (+/−)3-epi CP 47,497 | rel-2[(1S,3S)-3-hydroxycyclohexyl)]-5-(2- |
| | | methyloctan-2-yl)phenol |
| 37 | UR-144 N-(3- | (1-(3-chloropentyl)-1H-indol-3-yl)(2,2,3,3- |
| | chloropentyl) analog | tetramethylcyclopropyl)methanone |
| 38 | BB-22 | 1-(cyclohexylmethyl)-8-quinolinyl ester-1H- |
| | | indole-3-carboxylic acid |
| 39 | (+)-CP 47,497 | 2[(1R,3S)-3-hydroxycyclohexyl]-5-(2- |
| | | methyloctan-2-yl)phenol |
| 40 | AM2201 | (1-(5-fluoropentyl)-1H-benzo[d]imidazol-2- |
| | benzimidazole analog | yl)(naphthalen-1-yl)methanone |
| 41 | JWH 073 N-(2- | (1-isobutyl-1H-indol-3-yl)(naphthalen-1- |
| | methylpropyl) isomer | yl)methanone |
| 42 | RCS-8 3-methoxy | 1-(1-(2-cyclohexylethyl)-1H-indol-3-yl)-2-(3- |
| | isomer | methoxyphenyl)ethanone |
| 43 | MN-25 | 7-methoxy-1-[2-(4-morpholinyl)ethyl]-N- |
| | | [(1S,2 S,4R)-1,3,3-trimethylbicyclo[2.2.1] hept-2- |
| | | yl]-1H-indole-3-carboxamide |
| 44 | RCS-8 4-methoxy | 1-(1-(2-cyclohexylethyl)-1H-indol-3-yl)-2-(4- |
| | isomer | methoxyphenyl)ethanone |
| 45 | 5-fluoro PB-22 N-(3- | quinolin-8-yl 1-(3-fluoropentyl)-1H-indole-3- |
| | fluoropentyl) isomer | carboxylate |
| 46 | MDMB-FUBINACA | N-[[1-[(4-fluorophenyl)methyl]-1H-indazol-3- |
| | | yl]carbonyl]-3-methyl-1-valine, methyl ester |
| 47 | BB-22 7-HO-quinoline | quinolin-7-yl 1-(cyclohexylmethyl)-1H-indole-3- |
| | isomer | carboxylate |
| 48 | JWH 018 N-(1- | naphthalen-1-yl(1-(pentan-3-yl)-1H-indol-3- |
| | ethylpropyl) isomer | yl)methanone |
| 49 | JWH 018 N-(2,2- | naphthalen-1-yl(1-neopentyl-1H-indol-3-yl)- |
| | dimethylpropyl) isomer | methanone |
| 50 | JWH 398 8-Cl- | (8-chloronaphthalen-1-yl)(1-pentyl-1H-indol-3- |
| | naphthyl isomer | yl)methanone |
| 51 | XLR11 | (1-(5-fluoropentyl)-1H-indol-3-yl)(2,2,3,3- |
| | | tetramethylcyclopropyl)methanone |
| 52 | PB-22 4-HO- | isoquinolin-4-yl 1-pentyl-1H-indole-3-carboxylate |
| | isoquinoline isomer | |
| 53 | PB-22 6-HO- | isoquinolin-6-yl 1-pentyl-1H-indole-3-carboxylate |
| | isoquinoline isomer | |
| 54 | AM694 | [1-(5-fluoropentyl)-1H-indol-3-yl](2-iodophenyl)- |
| | | methanone |
| 55 | AM2232 | 3-(1-naphthalenylcarbonyl)-1H-Indole-1- |
| | | pentanenitrile |
| 56 | RCS-4 3-methoxy | (3-methoxyphenyl)(1-pentyl-1H-indol-3- |
| | isomer | yl)methanone |
| 57 | JWH 200 | [1-[2-(4-morpholinyl)ethyl]-1H-indol-3-yl]-1- |
| | | naphthalenyl-methanone |
| 58 | JWH 018 N-(2- | N-(2-methylbutyl)-3-(1-naphthoyl)-indole |
| | methylbutyl) isomer | |
| 59 | JWH 201 | 2-(4-methoxyphenyl)-1-(1-pentyl-1H-indol-3-yl)- |
| | | ethanone |
| 60 | JWH 370 | [5-(2-methylphenyl)-1-pentyl-1H-pyrrol-3-yl]-1- |
| | | naphthalenyl-methanone |
| 61 | CP 47,497-para- | 3′R-hydroxy-4-(2-methyloctan-2-yl)-[1,1′S- |
| | quinone analog | bi(cyclohexane)]-3,6-diene-2,5-dione |
| 62 | FUB-144 | (1-(4-fluorobenzyl)-1H-indol-3-yl)(2,2,3,3- |
| | | tetramethylcyclopropyl)methanone |
| 63 | JWH 369 | [5-(2-chlorophenyl)-1-pentyl-1H-pyrrol-3-yl]-1- |
| | | naphthalenyl-methanone |
| 64 | JWH 203 4-Cl-phenyl | 2-(4-chlorophenyl)-1-(1-pentyl-1H-indol-3-yl)- |
| | isomer | ethanone |
| 65 | JWH 072 | 1-naphthalenyl(1-propyl-1H-indol-3-yl)- |
| | | methanone |
| 66 | JWH 030 | 1-naphthalenyl(1-pentyl-1H-pyrrol-3-yl)- |
| | | methanone |
| 67 | AB-CHMINACA | N-[(1S)-1-(aminocarbonyl)-2-methylpropyl]-1- |
| | | (cyclohexylmethyl)-1H-indazole-3-carboxamide |
| 68 | AM1248 | [1-[(1-methyl-2-piperidinyl)methyl]-1H-indol-3- |
| | | yl]tricyclo[3.3.1.13,7]dec-1-yl-methanone |
| 69 | JWH 073 4-CH₃- | (1-butyl-1H-indol-3-yl)(4-methylnaphthalen-1- |
| | naphthyl analog | yl)methanone |
| 70 | JWH 018 N-(1,2- | (1-(3-methylbutan-2-yl)-1H-indol-3- |
| | dimethylpropyl) isomer | yl)(naphthalen-1-yl)methanone |
| 71 | JWH 018 | (1-pentyl-1H-indol-3-yl)-1-naphthalenyl- |
| | | methanone |
| 72 | JWH 031 | (1-hexyl-1H-pyrrol-3-yl)-1-naphthalenyl- |
| | | methanone |
| 73 | JWH 022 | 1-naphthalenyl[1-(4-penten-1-yl)-1H-indol-3-yl]- |
| | | methanone |
| 74 | RCS-8 | 1-(1-(2-cyclohexylethyl)-1H-indol-3-yl)-2-(2- |
| | | methoxyphenyl)ethanone |
| 75 | JWH 398 2-Cl- | (2-chloronaphthalen-1-yl)(1-pentyl-1H-indol-3- |
| | naphthyl isomer | yl)methanone |
| 76 | ADB-PINACA | N-[1-(aminocarbonyl)-2,2-dimethylpropyl]-1- |
| | | pentyl-1H-indazole-3-carboxamide |
| 77 | JWH 145 | 1-naphthalenyl(1-pentyl-5-phenyl-1H-pyrrol-3- |
| | | yl)-methanone |
| 78 | JWH 180 | (1-propyl-1H-indol-3-yl)(4-propyl-1- |
| | | naphthalenyl)-methanone |
| 79 | AM2201 | [1-(5-fluoropentyl)-1H-indol-3-yl]-1- |
| | | naphthalenyl-methanone |
| 80 | JWH 167 | 1-(1-pentyl-1H-indol-3-yl)-2-phenyl-ethanone |
| 81 | JWH 122 8-CH₃- | (8-methylnaphthalen-1-yl)(1-pentyl-1H-indol-3- |
| | naphthyl isomer | yl)methanone |
| 82 | JWH 147 | (1-hexyl-5-phenyl-1H-pyrrol-3-yl)-1- |
| | | naphthalenyl-methanone |
| 83 | 5-fluoro PB-22 N-(2-F- | quinolin-8-yl 1-(2-fluoropentyl)-1H-indole-3- |
| | pentyl) isomer | carboxylate |
| 84 | 4-fluoro ADB | methyl (2S)-2-(1-(4-fluoropentyl)-1H-indazole-3- |
| | | carboxamido)-3,3-dimethylbutanoate |
| 85 | JWH 307 | [5-(2-fluorophenyl)-1-pentyl-1H-pyrrol-3-yl]-1- |
| | | naphthalenyl-methanone |
| 86 | JWH203 3-Cl-phenyl | 2-(3-chlorophenyl)-1-(1-pentyl-1H-indol-3-yl)- |
| | isomer | ethanone |
| 87 | JWH424 | (8-bromonaphthalen-1-yl)(1-pentyl-1H-indol-3- |
| | | yl)methanone |
| 88 | ADBICA | N-(1-amino-3,3-dimethyl-1-oxobutan-2-yl)-1- |
| | | pentyl-1H-indole-3-carboxamide |
| 89 | JWH210 2-Et-naphthyl | (2-ethylnaphthalen-1-yl)(1-pentyl-1H-indol-3- |
| | isomer | yl)methanone |
| 90 | JWH 081 2-CH₃O- | (2-methoxy-1-naphthalenyl)(1-pentyl-1H-indol-3- |
| | naphthyl isomer | yl)-methanone |
| 91 | SDB-006 | 1-pentyl-N-(phenylmethyl)-1H-indole-3- |
| | | carboxamide |
| 92 | 5-fluoro PB-22 4-HO- | quinolin-4-yl 1-(5-fluoropentyl)-1H-indole-3- |
| | quinoline isomer | carboxylate |
| 93 | AM1248 azepane | (3,5,7)-adamantan-1-yl(1-(1-methylazepan-3-yl)- |
| | isomer | 1H-indol-3-yl)methanone |
| 94 | UR-144 | (1-pentyl-1H-indol-3-yl)(2,2,3,3- |
| | | tetramethylcyclopropyl)-methanone |
| 95 | A-836339 | [N(Z)]-N-[3-(2-methoxyethyl)-4,5-dimethyl- |
| | | 2(3H)-thiazolylidene]-2,2,3,3-tetramethyl- |
| | | cyclopropanecarboxamide |
| 96 | AB-CHMICA | (S)-N-(1-amino-3-methyl-1-oxobutan-2-yl)-1- |
| | | (cyclohexylmethyl)-1H-indole-3-carboxamide |
| 97 | 5-bromo THJ 018 | (1-(5-bromopentyl)-1H-indazol-3-yl)(naphthalen- |
| | | 1-yl)methanone |
| 98 | JWH 007 | (2-methyl-1-pentyl-1H-indol-3-yl)-1- |
| | | naphthalenyl-methanone |
| 99 | PB-22 5-HO- | isoquinolin-5-yl 1-pentyl-1H-indole-3-carboxylate |
| | isoquinoline isomer | |
| 100 | AB-PINACA | (S)-N-(1-amino-3-methyl-1-oxobutan-2-yl)-1- |
| | | pentyl-1H-indazole-3-carboxamide |
| 101 | JWH 251 | 2-(2-methyl phenyl)-1-(1-pentyl-1H-indol-3-yl)- |
| | | ethanone |
| 102 | 5-fluoro PB-22 N-(4-F- | quinolin-8-yl 1-(4-fluoropentyl)-1H-indole-3- |
| | pentyl) isomer | carboxylate |
| 103 | JWH 018 N-(1,1- | naphthalen-1-yl(1-(tert-pentyl)-1H-indol-3- |
| | dimethylpropyl) isomer | yl)methanone |
| 104 | AM2201 N-(2-F- | (1-(2-fluoropentyl)-1H-indol-3-yl)(naphthalen-1- |
| | pentyl) isomer | yl)methanone |
| 105 | 5-fluoro PB-22 5-HO- | isoquinolin-5-yl 1-(5-fluoropentyl)-1H-indole-3- |
| | isoquinoline isomer | carboxylate |
| 106 | JWH 250 | 1-(1-pentyl-1H-indol-3-yl)-2-(2-methoxyphenyl)- |
| | | ethanone |
| 107 | JWH 073 6-CH₃O- | (1-butyl-6-methoxy-1H-indol-3-yl)-1- |
| | indole analog | naphthalenyl-methanone |
| 108 | JWH 018 adamantyl | 1-pentyl-N-tricyclo[3.3.1.1]dec-1-yl-1H-indole-3- |
| | carboxamide | carboxamide |
| 109 | JWH 368 | [5-(3-fluorophenyl)-1-pentyl-1H-pyrrol-3-yl]-1- |
| | | naphthalenyl-methanone |
| 110 | XLR12 | (2,2,3,3-tetramethylcyclopropyl)[1-(4,4,4- |
| | | trifluorobutyl)-1H-indol-3-yl]-methanone |
| 111 | Azidoindolene 1 | 2,2,3,3-tetramethyl-cyclopropanecarboxylic acid |
| | | (2Z)-2-[1-(5-fluoropentyl)-1,2-dihydro-2-oxo-3H- |
| | | indol-3-ylidene]hydrazide |
| 112 | F2201 | (4-fluoro-1-naphthalenyl)[1-(5-fluoropentyl)-1H- |
| | | indol-3-yl]-methanone |
| 113 | UR-144 N-(4-Cl- | (1-(4-chloropentyl)-1H-indol-3-yl)(2,2,3,3- |
| | pentyl) analog | tetramethylcyclopropyl)methanone |
| 114 | AM2201 N-(3-Cl- | (1-(3-chloropentyl)-1H-indol-3-yl)(naphthalen-1- |
| | pentyl) isomer | yl)methanone |
| 115 | JWH 210 8-Et- | (8-ethylnaphthalen-1-yl)(1-pentyl-1H-indol-3- |
| | naphthyl isomer | yl)methanone |
| 116 | 5-Fluoropentyl-3- | (1-(5-fluoropentyl)-1H-indol-3-yl)(pyridin-3- |
| | pyridinoylindole | yl)methanone, monohydrochloride |
| 117 | AKB48 N-(4-F-benzyl) | N-((3s,5s,7s)-adamantan-1-yl)-1-(4-fluorobenzyl)- |
| | analog | 1H-indazole-3-carboxamide |
| 118 | UR-144 N-heptyl | (1-heptyl-1H-indol-3-yl)(2,2,3,3- |
| | analog | tetramethylcyclopropyl)-methanone |
| 119 | 5-fluoro-3,5-ADB- | N-[1-(aminocarbonyl)-2,2-dimethylpropyl]-1-(5- |
| | PFUPPYCA | fluoropentyl)-3-(4-fluorophenyl)-1H-pyrazole-5- |
| | | carboxamide |
| 120 | JWH 249 | 2-(2-bromophenyl)-1-(1-pentyl-1H-indol-3-yl)- |
| | | ethanone |
| 121 | IMMA | 2-[1-(4-chlorobenzoyl)-5-methoxy-2-methyl-1H- |
| | | indol-3-yl]-1-(4-morpholinyl)-ethanone |
| 122 | JWH 016 | (1-butyl-2-methyl-1H-indol-3-yl)-1-naphthalenyl- |
| | | methanone |
| 123 | JWH 018 | naphthalen-1-yl(1-pentyl-1H-benzo[d]imidazol-2- |
| | benzimidazole analog | yl)methanone |
| 124 | BB-22 4-HO- | isoquinolin-4-yl 1-(cyclohexylmethyl)-1H-indole- |
| | isoquinoline isomer | 3-carboxylate |
| 125 | MDA 19 | (2Z)-2-(1-hexyl-1,2-dihydro-2-oxo-3H-indol-3- |
| | | ylidene)hydrazide, benzoic acid |
| 126 | M-144 | (1-(5-fluoropentyl)-2-methyl-1H-indol-3- |
| | | yl)(2,2,3,3-tetramethylcyclopropyl)methanone |
| 127 | JWH 073 N-(1-CH₃- | (1-(sec-butyl)-1H-indol-3-yl)(naphthalen-1- |
| | propyl) isomer | yl)methanone |
| 128 | AM2201 N-(3- | (1-(3-fluoropentyl)-1H-indol-3-yl)(naphthalen-1- |
| | fluoropentyl) isomer | yl)methanone |
| 129 | RCS-4 | (4-methoxyphenyl)(1-pentyl-1H-indol-3- |
| | | yl)methanone |
| 130 | AM694 3-iodo isomer | (1-(5-fluoropentyl)-1H-indol-3-yl)(3- |
| | | iodophenyl)methanone |
| 131 | PB-22 5-HO-quinoline | quinolin-5-yl 1-pentyl-1H-indole-3-carboxylate |
| | isomer | |
| 132 | JWH 200 analog 1 | (E)-3,4,4-trimethyl-1-(1-(2-morpholinoethyl)-1H- |
| | | indol-3-yl)pent-2-en-1-one |
| 133 | XLR11 N-(3- | [1-(3-fluoropentyl)-1H-indol-3-yl] (2,2,3,3- |
| | fluoropentyl) isomer | tetramethylcyclopropyl)-methanone |
| 134 | THJ2201 | [1-(5-fluoropentyl)-1H-indazol-3-yl]-1- |
| | | naphthalenyl-methanone |
| 135 | AB-FUBINACA | N-(1-amino-1-oxobutan-2-yl)-1-(4-fluorobenzyl)- |
| | isomer 5 | N-methyl-1H-indazole-3-carboxamide |
| 136 | 5-fluoro PB-22 3-HO- | quinolin-3-yl 1-(5-fluoropentyl)-1H-indole-3- |
| | quinoline isomer | carboxylate |
| 137 | JWH 011 | [2-methyl-1-(1-methylhexyl)-1H-indol-3-yl]-1- |
| | | naphthalenyl-methanone |
| 138 | UR-144 N-(5- | (1-(5-chloropentyl)-1H-indol-3-yl)(2,2,3,3- |
| | chloropentyl) analog | tetramethylcyclopropyl)methanone |
| 139 | AM2201 2′-naphthyl | (1-(5-fluoropentyl)-1H-indol-3-yl)(naphthalen-2- |
| | isomer | yl)methanone |
| 140 | JWH 019 | (1-hexyl-1H-indol-3-yl)-1-naphthalenyl- |
| | | methanone |
| 141 | JWH 018 8-quinolinyl | 1-pentyl-N-8-quinolinyl-1H-indole-3-carboxamide |
| | carboxamide | |
| 142 | 5-chloro THJ 018 | (1-(5-chloropentyl)-1H-indazol-3-yl)(naphthalen- |
| | | 1-yl)methanone |
| 143 | AB-FUBINACA 2-F- | N-[(1S)-1-(aminocarbonyl)-2-methylpropyl]-1- |
| | benzyl isomer | [(2-fluorophenyl)methyl]-1H-indazole-3- |
| | | carboxamide |
| 144 | STS-135 | 1-(5-fluoropentyl)-N-tricyclo[3.3.1.13,7] dec-1-yl- |
| | | 1H-indole-3-carboxamide |
| 145 | MAM2201 N-(4- | (1-(4-fluoropentyl)-1H-indol-3-yl)(4- |
| | fluoropentyl) isomer | methylnaphthalen-1-yl)methanone |
| 146 | JWH 398 7-Cl- | (7-chloronaphthalen-1-yl)(1-pentyl-1H-indol-3- |
| | naphthyl isomer | yl)methanone |
| 147 | A-796260 | [1-[2-(4-morpholinyl)ethyl]-1H-indol-3- |
| | | yl](2,2,3,3-tetramethylcyclopropyl)-methanone |
| 148 | XLR11 N-(4-pentenyl) | (1-(pent-4-en-1-yl)-1H-indol-3-yl)(2,2,3,3- |
| | analog | tetramethylcyclopropyl)methanone |
| 149 | THJ 018 | 1-naphthalenyl(1-pentyl-1H-indazol-3-yl)- |
| | | methanone |
| 150 | JWH 412 | (4-fluoro-1-naphthalenyl)(1-pentyl-1H-indol-3- |
| | | yl)-methanone |
| 151 | ADB-PINACA | N-(1-amino-2,3-dimethyl-1-oxobutan-2-yl)-1- |
| | isomer 1 | pentyl-1H-indazole-3-carboxamide |
| 152 | CB-13 | 1-naphthalenyl[4-(pentoxy)-1-naphthalenyl]- |
| | | methanone |
| 153 | XLR11 N-(2- | [1-(2-fluoropentyl)-1H-indol-3-yl] (2,2,3,3- |
| | fluoropentyl) isomer | tetramethylcyclopropyl)-methanone |
| 154 | JWH 116 | (2-ethyl-1-pentyl-1H-indol-3-yl)-1-naphthalenyl- |
| | | methanone |
| 155 | MAM2201 | [1-(5-fluoropentyl)-1H-indol-3-yl](4-methyl-1- |
| | | naphthalenyl)-methanone |
| 156 | JWH 018 | N-(4,5-epoxypentyl) analog naphthalen-1-yl(1-(3- |
| | | (oxiran-2-yl)propyl)-1H-indol-3-yl)methanone |
| 157 | UR-144 N-(2- | (1-(2-chloropentyl)-1H-indol-3-yl)(2,2,3,3- |
| | chloropentyl) analog | tetramethylcyclopropyl)methanone |
| 158 | 5-fluoro PB-22 5-HO- | quinolin-5-yl 1-(5-fluoropentyl)-1H-indole-3- |
| | quinoline isomer | carboxylate |
| 159 | CB-86 | N-cyclopropyl-8-[3-(1,1-dimethylheptyl)-5- |
| | | hydroxyphenoxy]-octanamide |
| 160 | 5-fluoro JWH 018 | [1-(5-fluoropentyl)-1H-indol-3- |
| | adamantyl analog | yl]tricyclo[3.3.1.13,7]dec-1-yl-methanone |
| 161 | JWH 122 7-CH₃- | (7-methyl-1-naphthalenyl)(1-pentyl-1H-indol-3- |
| | naphthyl isomer | yl)-methanone |
| 162 | BB-22 5-HO- | isoquinolin-5-yl 1-(cyclohexylmethyl)-1H-indole- |
| | isoquinoline isomer | 3-carboxylate |
| 163 | JWH 081 7-CH₃O- | (7-methoxy-1-naphthalenyl)(1-pentyl-1H-indol-3- |
| | naphthyl isomer | yl)-methanone |
| 164 | JWH 182 | (1-pentyl-1H-indol-3-yl)(4-propyl-1- |
| | | naphthalenyl)-methanone |
| 165 | JWH 251 3-CH₃- | 1-(1-pentyl-1H-indol-3-yl)-2-(m-tolyl)ethanone |
| | phenyl isomer | |
| 166 | 5-fluoro THJ | 1-(5-fluoropentyl)-N-(quinolin-8-yl)-1H-indazole- |
| | | 3-carboxamide |
| 167 | 5-fluoro SDB-005 | naphthalen-1-yl 1-(5-fluoropentyl)-1H-indazole-3 - |
| | | carboxylate |
| 168 | JWH 018 2′-naphthyl- | [1-(2,2-dimethylpropyl)-1H-indol-3-yl]-2- |
| | N-(2,2-dimethylbutyl) | naphthalenyl-methanone |
| | isomer | |
| 169 | AB-FUBINACA | N-(1-amino-1-oxopentan-2-yl)-1-(4-fluorobenzyl)- |
| | isomer 1 | 1H-indazole-3-carboxamide |
| 170 | 5-chloro AB-PINACA | N-[(1S)-1-(aminocarbonyl)-2-methylpropyl]-1-(5- |
| | | chloropentyl)-1H-indazole-3-carboxamide |
| 171 | SDB-005 | naphthalen-1-yl 1-pentyl-1H-indazole-3 - |
| | | carboxylate |
| 172 | AB-FUBINACA | N-[(1S)-1-(aminocarbonyl)-2-methylpropyl]-1- |
| | | [(4-fluorophenyl)methyl]-1H-indazole-3- |
| | | carboxamide |
| 173 | JWH 019 N-(4- | (1-(4-fluorohexyl)-1H-indol-3-yl)(naphthalen-1- |
| | fluorohexyl) isomer | yl)methanone |
| 174 | MAM2201 N-(3- | (1-(3-fluoropentyl)-1H-indol-3-yl)(4- |
| | fluoropentyl) isomer | methylnaphthalen-1-yl)methanone |
| 175 | JWH 018 6-CH3O- | (6-methoxy-1-pentyl-1H-indol-3-yl)(naphthalen-1- |
| | indole analog | yl)-methanone |
| 176 | JWH 019 N-(3- | (1-(3-fluorohexyl)-1H-indol-3-yl)(naphthalen-1- |
| | fluorohexyl) isomer | yl)methanone |
| 177 | JWH 018 N-(5- | (1-(5-chloropentyl)-1H-indol-3-yl)(naphthalen-1- |
| | chloropentyl) analog | yl)methanone |
| 178 | JWH 030 2-naphthoyl | naphthalen-1-yl(1-pentyl-1H-pyrrol-2- |
| | isomer | yl)methanone |
| 179 | 5-fluoro ADB | N-[[1-(5-fluoropentyl)-1H-indazol-3-yl] carbonyl]- |
| | | 3-methyl-D-valine, methyl ester |
| 180 | JWH 210 | (4-ethyl-1-naphthalenyl)(1-pentyl-1H-indol-3-yl)- |
| | | methanone |
| 181 | JWH 251 4-CH₃- | 2-(4-methylphenyl)-1-(1-pentyl-1H-indol -3-yl)- |
| | phenyl isomer | ethanone |
| 182 | JP104 | 3′-carbamoyl-biphenyl-3-yl-undecynecarbamate |
| 183 | AMB | N-[(1-pentyl-1H-indazol-3-yl)carbonyl]-1-valine, |
| | | methyl ester |
| 184 | 5-fluoro MN-18 | 1-(5-fluoropentyl)-N-1-naphthalenyl-1H-indazole- |
| | | 3-carboxamide |
| 185 | FAB-144 | (1-(5-fluoropentyl)-1H-indazol-3-yl)(2,2,3,3- |
| | | tetramethylcyclopropyl)methanone |
| 186 | ADB-BICA | N-(1-amino-3,3-dimethyl-1-oxobutan-2-yl)-1- |
| | | benzyl-1H-indole-3-carboxamide |
| 187 | JWH 018 N-(1- | naphthalen-1-yl(1-(pentan-2-yl)-1H-indol -3- |
| | methylbutyl) isomer | yl)methanone |
| 188 | JWH 133 | 3-(1,1-dimethylbutyl)-6aR,7,10,10aR-tetrahydro- |
| | | 6,6,9-trimethyl-6H-dibenzo[b,d]pyran |
| 189 | MAM2201 N-(5- | (1-(5-chloropentyl)-1H-indol-3-yl)(4- |
| | chloropentyl) analog | methylnaphthalen-1-yl)methanone |
| 190 | JWH 203 | 2-(2-chlorophenyl)-1-(1-pentyl-1H-indol -3-yl)- |
| | | ethanone |
| 191 | JWH 122 5-CH3- | (5-methylnaphthalen-1-yl)(1-pentyl-1H-indol-3- |
| | naphthyl isomer | yl)methanone |
| 200 | PSB-SB1202 | 5-methoxy-3-[(2-methoxyphenyl)methyl]-7- |
| | | pentyl-2H-1-benzopyran-2-one |
| 201 | JWH 018 2′-naphthyl- | [1-(1-ethylpropyl)-1H-indol-3-yl]-2-naphthalenyl- |
| | N-(1-ethylpropyl) | methanone |
| | isomer | |
| 202 | JWH 031 2′-isomer | (1-hexyl-1H-pyrrol-2-yl)-1-naphthalenyl- |
| | | methanone |
| 203 | JWH 193 | (4-methyl-1-naphthalenyl)[1-[2-(4- |
| | | morpholinyl)ethyl]-1H-indol-3-yl]-methanone |
| 204 | FUB-PB-22 | 1-[(4-fluorophenyl)methyl]-1H-indole-3- |
| | | carboxylic acid, 8-quinolinyl ester |
| 205 | JWH 387 | (4-bromo-1-naphthalenyl)(1-pentyl-1H-indol-3- |
| | | yl)-methanone |
| 206 | BB-22 5-HO-quinoline | quinolin-5-yl 1-(cyclohexylmethyl)-1H-indole-3- |
| | isomer | carboxylate |
| 207 | JWH 019 N-(2- | (1-(2-fluorohexyl)-1H-indol-3-yl)(naphthalen-1- |
| | fluorohexyl) isomer | yl)methanone |
| 208 | AM2201 8-quinolinyl | 1-(5-fluoropentyl)-N-(quinolin-8-yl)-1H-indole-3- |
| | carboxamide | carboxamide |
| 209 | CP 47,497-C6- | cis-5-(1,1-dimethylhexyl)-2-(3- |
| | homolog | hydroxycyclohexyl)-phenol |
| 210 | 3-fluoro AMB | methyl (1-(3-fluoropentyl)-1H-indazole-3- |
| | | carbonyl)-1-valinate |
| 211 | JWH 122 | (4-methyl-1-naphthalenyl)(1-pentyl-1H-indol-3- |
| | | yl)-methanone |
| 212 | JWH 146 | (1-heptyl-5-phenyl-1H-pyrrol-3-yl)-1- |
| | | naphthalenyl-methanone |
| 213 | APP-FUBINACA | N-[(1S)-2-amino-2-oxo-1-(phenylmethyl)ethyl]-1- |
| | | [(4-fluorophenyl)methyl]-1H-indazole-3- |
| | | carboxamide |
| 214 | JWH 309 | 1-naphthalenyl[5-(1-naphthalenyl)-1-pentyl-1H- |
| | | pyrrol-3-yl]-methanone |
| 215 | 5-fluoro PY-PICA | (1-(5-fluoropentyl)-1H-indol-3-yl)(pyrrolidin-1- |
| | | yl)methanone |
| 216 | EAM2201 | (4-ethyl-1-naphthalenyl)[1-(5-fluoropentyl)-1H- |
| | | indol-3-yl]-methanone |
| 217 | ADB-BINACA | N-(1-amino-3,3-dimethyl-1-oxobutan-2-yl)-1- |
| | | benzyl-1H-indazole-3-carboxamide |
| 218 | 5-fluoro PB-22 6-HO- | isoquinolin-6-yl 1-(5-fluoropentyl)-1H-indole-3- |
| | isoquinoline isomer | carboxylate |
| 219 | JWH 122 3-CH₃- | (3-methylnaphthalen-1-y1)(1-pentyl-1H-indol-3- |
| | naphthyl isomer | yl)methanone |
| 220 | FUB-JWH 018 | (1-(4-fluorobenzyl)-1H-indol-3-yl)(naphthalen-1- |
| | | yl)methanone |
| 221 | AB-FUBINACA 3-F- | N-[(1S)-1-(aminocarbonyl)-2-methylpropyl]-1- |
| | benzyl isomer | [(3-fluorophenyl)methyl]-1H-indazole-3- |
| | | carboxamide |
| 222 | UR-144 N-(5- | [1-(5-bromopentyl)-1H-indol-3-yl](2,2,3,3- |
| | bromopentyl) analog | tetramethylcyclopropyl)-methanone |
| 223 | JWH 398 | (4-chloro-1-naphthalenyl)(1-pentyl-1H-indol-3- |
| | | yl)-methanone |
| 224 | MN-18 | N-1-naphthalenyl-1-pentyl-1H-indazole-3- |
| | | carboxamide |
| 225 | UR-144 N-(5- | (1-(5-methylhexyl)-1H-indol-3-yl)(2,2,3,3 - |
| | methylhexyl) analog | tetramethylcyclopropyl)methanone |
| 226 | URB602 | N-[1,1′-biphenyl]-3-yl-carbamic acid, cyclohexyl |
| | | ester |
| 227 | PX 2 | (S)-N-(1-amino-1-oxo-3-phenylpropan-2-yl)-1-(5- |
| | | fluoropentyl)-1H-indazole-3-carboxamide |
| 228 | 5-fluoro CUMYL- | 1-(5-fluoropentyl)-N-(1-methyl-1-phenyl ethyl)- |
| | PINACA | 1H-indazole-3-carboxamide |
| 229 | MMB-CHMICA | methyl (1-(cyclohexylmethyl)-1H-indole-3- |
| | | carbonyl)-1-valinate |
| 230 | 5-fluoro PY-PINACA | (1-(5-fluoropentyl)-1H-indazol-3-yl)(pyrrolidin-1- |
| | | yl)methanone |
| 231 | JWH 210 7-Et- | (7-ethyl-1-naphthalenyl)(1-pentyl-1H-indol-3-yl)- |
| | naphthyl isomer | methanone |
| 232 | THJ2201 | 1-pentyl-N-(quinolin-8-yl)-1H-indazole-3- |
| | | carboxamide |
| 233 | JWH 019 N-(5- | (1-(5-fluorohexyl)-1H-indol-3-yl)(naphthalen-1- |
| | fluorohexyl) isomer | yl)methanone |
| 234 | JWH081 3-CH₃O- | (3-methoxynaphthalen-1-yl)(1-pentyl-1H-indol-3- |
| | naphthyl isomer | yl)methanone |
| 235 | XLR11 N-(4- | (1-(4-fluoropentyl)-1H-indol-3-yl)(2,2,3,3- |
| | fluuoropentyl) isomer | tetramethylcyclopropyl)methanone |
| 236 | 5-fluoro AEB | ethyl (1-(5-fluoropentyl)-1H-indazole-3-carbonyl)- |
| | | L-valinate |
| 237 | AB-FUBICA | N-[(1S)-1-(aminocarbonyl)-2-methylpropyl]-1- |
| | | [(4-fluorophenyl)methyl]-1H-indole-3- |
| | | carboxamide |
| 238 | JWH 398 5-Cl- | (5-chloronaphthalen-1-yl)(1-pentyl-1H-indol-3- |
| | naphthyl isomer | yl)methanone |
| 239 | MDMB-CHMCZCA | methyl (S)-2-(9-(cyclohexylmethyl)-9H-carbazole- |
| | | 3-carboxamido)-3,3-dimethylbutanoate |
| 240 | JWH 122 6-CH₃- | (6-methylnaphthalen-1-yl)(1-pentyl-1H-indol-3- |
| | naphthyl isomer | yl)methanone |
| 241 | (+/−)-CP 47,497-C8- | rel-5-(1,1-dimethyloctyl)-2-[(1R,3S)-3- |
| | homolog | hydroxycyclohexyl]-phenol |
| 242 | AM3102 | N-[(1R)-2-hydroxy-1-methylethyl-9Z- |
| | | octadecenamide |
| 243 | APINAC | (3s,5s,7s)-adamantan-1-yl 1-pentyl-1H-indazole-3- |
| | | carboxylate |
| 244 | JWH 213 | (4-ethyl-1-naphthalenyl)(2-methyl-1-pentyl-1H- |
| | | indol-3-yl)-methanone |
| 245 | JWH 018 adamantyl | (1s,3s)-adamantan-1-yl(1-pentyl-1H-indol-3- |
| | analog | yl)methanone |
| 246 | APP-PICA | (S)-N-(1-amino-1-oxo-3-phenylpropan-2-yl)-1- |
| | | pentyl-1H-indole-3-carboxamide |
| 247 | ADB-FUBINACA | N-[1-(aminocarbonyl)-2,2-dimethylpropyl]-1-[(4- |
| | | fluorophenyl)methyl]-1H-indazole-3-carboxamide |
| 248 | FDU-PB-22 | 1-[(4-fluorophenyl)methyl]-1H-indole-3- |
| | | carboxylic acid, 1-naphthalenyl ester |
| 249 | CBL-018 | naphthalen-1-yl 1-pentyl-1H-indole-3 -carboxylate |
| 250 | Flurazepam | 7-chloro-1-[2-(diethylamino)ethyl]-5-(2- |
| | | fluorophenyl)-1,3-dihydro-2H-1,4-benzodiazepin- |
| | | 2-one |
| 251 | JWH 018 2′-naphthyl- | (1-isopentyl-1H-indol-3-yl)(naphthalen-2- |
| | N-(3-methylbutyl) | yl)methanone |
| | isomer | |
| 252 | JWH 175 | 3-(1-naphthalenylmethyl)-1-pentyl-1H-indole |
| 253 | JWH 081 N- | [1-(cyclohexylmethyl)-1H-indol-3-yl](4-methoxy- |
| | (cyclohexylmethyl) | 1-naphthalenyl)-methanone |
| | analog | |
| 254 | BB-22 7-HO- | isoquinolin-7-yl 1-(cyclohexylmethyl)-1H-indole- |
| | isoquinoline isomer | 3-carboxylate |
| 255 | JWH 210 5-Et- | (5-ethyl-1-naphthalenyl)(1-pentyl-1H-indol-3-yl)- |
| | naphthyl isomer | methanone |
| 256 | PB-22 6-HO-quinoline | quinolin-6-yl 1-pentyl-1H-indole-3-carboxylate |
| | isomer | |
| 257 | NM2201 | naphthalen-1-yl 1-(5-fluoropentyl)-1H-indole-3- |
| | | carboxylate |
| 258 | NPB-22 | 1-pentyl-1H-indazole-3-carboxylic acid, 8- |
| | | quinolinyl ester |
| 259 | JWH 018 2′-naphthyl | 2-naphthalenyl(1-pentyl-1H-indol-3-yl)- |
| | isomer | methanone |
| 260 | A-834735 | [1-[(tetrahydro-2H-pyran-4-yl)methyl]-1H-indol- |
| | | 3-yl](2,2,3,3-tetramethylcyclopropyl)-methanone |
| 261 | BB-22 3-HO-quinoline | Quinolin-3-yl 1-(cyclohexylmethyl)-1H-indole-3- |
| | isomer | carboxylate |
| 262 | (−)-CP 55,940 | 5-(1,1-dimethylheptyl)-2-[(1R,2R,5R)-5-hydroxy- |
| | | 2-(3-hydroxypropyl)cyclohexyl]-phenol |
| 263 | JWH 398 3-Cl- | (3-chloronaphthalen-1-yl)(1-pentyl-1H-indol-3- |
| | naphthyl isomer | yl)methanone, hydrate |
| | (hydrate) | |
| 264 | JWH 210 3-Et- | (3-ethyl-1-naphthalenyl)(1-pentyl-1H-indol-3-yl)- |
| | naphthyl isomer | methanone |
| 265 | JWH 081 | (4-methoxy-1-naphthalenyl)(1-pentyl-1H-indol-3- |
| | | yl)-methanone |
| 266 | MDA 77 | 2Z-(1,2-dihydro-6-methoxy-2-oxo-1-pentyl-3H- |
| | | indol-3-ylidene)hydrazide benzoic acid |
| 267 | PX 1 | (S)-N-(1-amino-1-oxo-3-phenylpropan-2-yl)-1-(5- |
| | | fluoropentyl)-1H-indole-3-carboxamide |
| 268 | 4-fluoro AMB | methyl (1-(4-fluoropentyl)-1H-indazole-3- |
| | | carbonyl)-1-valinate |
| 269 | MDMB-FUBICA | N-[[1-[(4-fluorophenyl)methyl]-1H-indol-3- |
| | | yl]carbonyl]-3-methyl-1-valine, methyl ester |
| 270 | MMB-FUBICA | N-[[1-[(4-fluorophenyl)methyl]-1H-indol-3- |
| | | yl]carbonyl]-1-valine, methyl ester |
| 271 | JWH 018 2′-naphthyl- | (1-(2-methylbutyl)-1H-indol-3-yl)(naphthalen-2- |
| | N-(2-methylbutyl) | yl)-methanone |
| | isomer | |
| 272 | JWH 071 | (1-ethyl-1H-indol-3-yl)-1-naphthalenyl- |
| | | methanone |
| 273 | Mepirapim | (4-methylpiperazin-1-yl)(1-pentyl-1H-indol-3- |
| | (hydrochloride) | yl)methanone, monohydrochloride |
| 274 | PB-22 7-HO- | isoquinolin-7-yl 1-pentyl-1H-indole-3-carboxylate |
| | isoquinoline isomer | |
| 275 | JWH 210 6-Et- | (6-ethylnaphthalen-1-yl)(1-pentyl-1H-indol-3- |
| | naphthyl isomer | yl)methanone |
| 276 | FUB-NPB-22 | quinolin-8-yl 1-(4-fluorobenzyl)-1H-indazole-3- |
| | | carboxylate |
| 277 | (+/−)-ORG 28611 | [1-(cyclohexylmethyl)-7-methoxy-1H-indol-3- |
| | | yl](3,4-dimethyl-1-piperazinyl)-methanone |
| 278 | LY2183240 2′-isomer | 5-([1,1′-biphenyl]-4-ylmethyl)-N,N-dimethyl-2H- |
| | | tetrazole-2-carboxamide |
| 279 | 5-fluoro AMB | N-[[1-(5-fluoropentyl)-1H-indazol-3-yl] carbonyl]- |
| | | L-valine, methyl ester |
| 280 | AM1235 | [1-(5-fluoropentyl)-6-nitro-1H-indol-3-yl]-1- |
| | | naphthalenyl-methanone |
| 281 | 4-cyano CUMYL- | 1-(4-cyanobutyl)-N-(1-methyl-1-phenyl ethyl)-1H- |
| | BUTINACA | indazole-3-carboxamide |
| 282 | RCS-4-C4 homolog | (4-methoxyphenyl)(1-butyl-1H-indol-3-yl)- |
| | | methanone |
| 283 | AM694 4-iodo isomer | (1-(5-fluoropentyl)-1H-indol-3-yl)(4- |
| | | iodophenyl)methanone |
| 284 | JWH 073 2′-naphthyl- | (1-isobutyl-1H-indol-3-yl)(naphthalen-2- |
| | N-(2-methylpropyl) | yl)methanone |
| | isomer | |
| 285 | JWH 398 6-Cl- | (6-chloronaphthalen-1-yl)(1-pentyl-1H-indol-3- |
| | naphthyl isomer | yl)methanone |
| 286 | 5-fluoro NNEI | 1-(5-fluoropentyl)-N-(naphthalen-1-yl)-1H-indole- |
| | | 3-carboxamide |
| 287 | AB-FUBINACA | N-(1-amino-2-methyl-1-oxobutan-2-yl)-1-(4- |
| | isomer 2 | fluorobenzyl)-1H-indazole-3-carboxamide |
| 288 | LY2183240 | 5-([1,1′-biphenyl]-4-ylmethyl)-N,N-dimethyl-1H- |
| | | tetrazole-1-carboxamide |
| 289 | 5-fluoro ADB- | N-[1-(aminocarbonyl)-2,2-dimethylpropyl]-1-(5- |
| | PINACA | fluoropentyl)-1H-indazole-3-carboxamide |
| 292 | 5-fluoro AB-PINACA | N-[(1S)-1-(aminocarbonyl)-2-methylpropyl]-1-(5- |
| | | fluoropentyl)-1H-indazole-3-carboxamide |
| 293 | AB-PINACA N-(4- | N-(1-amino-3-methyl-1-oxobutan-2-yl)-1-(4- |
| | fluoropentyl) isomer | fluoropentyl)-1H-indazole-3-carboxamide |
| 294 | NNEI 2′-indazole | N-(naphthalen-1-yl)-2-pentyl-2H-indazole-3- |
| | isomer | carboxamide |
| 295 | MN-25-2-methyl | 7-methoxy-2-methyl-1-[2-(4-morpholinyl)ethyl]- |
| | derivative | N-[(1S,2S,4R)-1,3,3-trimethylbicyclo[2.2.1]hept- |
| | | 2-yl]-1H-indole-3-carboxamide |
| 296 | 2-fluoro NNEI | 1-(2-fluoropentyl)-N-(naphthalen-1-yl)-1H-indole- |
| | | 3-carboxamide |
| 297 | 5-fluoro ADBICA | N-[1-(aminocarbonyl)-2,2-dimethylpropyl]-1-(5- |
| | | fluoropentyl)-1H-indole-3-carboxamide |
| 298 | 5-fluoro ADB- | N-((2S,3S)-1-amino-3-methyl-1-oxopentan-2-yl)- |
| | PINACA isomer 2 | 1-(5-fluoropentyl)-1H-indazole-3-carboxamide |
| 299 | 4-cyano CUMYL- | 2-(4-cyanobutyl)-N-(2-phenylpropan-2-yl)-2H- |
| | BUTINACA isomer 2 | indazole-3-carboxamide |
| 300 | JWH 081 6-CH₃O- | (6-methoxy-1-naphthalenyl)(1-pentyl-1H-indol-3- |
| | naphthyl isomer | yl)-methanone |
| 301 | (+/−)3-epi CP 47,497-C₈- | rel-2-[(1S,3S)-3-hydroxycyclohexyl)]-5-(2- |
| | homolog | methylnonan-2-yl)phenol |
| 302 | CB-52 | N-cyclopropyl-11-(2-hexyl-5-hydroxyphenoxy)- |
| | | undecanamide |
| 303 | JWH 176 | 1-[(E)-(3-pentyl-1H-inden-1-ylidene)methyl]- |
| | | naphthalene |
| 304 | HU-308 | 4-[4-(1,1-dimethylheptyl)-2,6-dimethoxyphenyl]- |
| | | 6,6-dimethyl-bicyclo[3.1.1]hept-2-ene-2-methanol |
| 305 | AB-005 azepane | (1-(1-methylazepan-3-yl)-1H-indol-3-yl)(2,2,3,3- |
| | isomer | tetramethylcyclopropyl)methanone |
| 306 | 5-fluoro SDB-006 | 1-(5-fluoropentyl)-N-(phenylmethyl)-1H-indole-3- |
| | | carboxamide |
| 307 | SDB-006 N-phenyl | 1-pentyl-N-phenyl-1H-indole-3-carboxamide |
| | analog | |
| 308 | Pravadoline | (4-methoxyphenyl)[2-methyl]-1-[2-(4- |
| | | morpholinyl)ethyl]-1H-indol-3-yl]-methanone |
| 309 | AKB48 N-(5- | N-((3s,5s,7s)-adamantan-1-yl)-1-(5-fluoropentyl)- |
| | fluoropentyl) analog | 1H-indazole-3-carboxamide |
| 310 | WIN 54,461 | [6-bromo-2-methyl-1-[2-(4-morpholinyl)ethyl]- |
| | | 1H-indol-3-yl](4-methoxyphenyl)-methanone |
| 311 | 5-fluoro NPB-22 | 1-(5-fluoropentyl)-8-quinolinyl ester-1H-indazole- |
| | | 3-carboxylic acid |
| 312 | 5-fluoro ABICA | N-[(1S)-1-(aminocarbonyl)-2-methylpropyl]-1-(5- |
| | | fluoropentyl)-1H-indole-3-carboxamide |
| 313 | 5-fluoro CUMYL- | 1-(5-fluoropentyl)-N-(2-phenylpropan-2-yl)-1H- |
| | P7AICA | pyrrolo[2,3-b] pyridine-3-carboxamide |
| 314 | BB-22 6-HO-quinoline | quinolin-6-yl 1-(cyclohexylmethyl)-1H-indole-3- |
| | isomer | carboxylate |
| 315 | EG 018 | naphthalen-1-yl(9-pentyl-9H-carbazol-3- |
| | | yl)methanone |
| 316 | AM2201 N-(4- | (1-(4-fluoropentyl)-1H-indol-3-yl)(naphthalen-1- |
| | fluoropentyl) isomer | yl)methanone |
| 317 | AB-PINACA N-(2- | N-(1-amino-3-methyl-1-oxobutan-2-yl)-1-(2- |
| | fluoropentyl) isomer | fluoropentyl)-1H-indazole-3-carboxamide |
| 318 | EG2201 | (9-(5-fluoropentyl)-9H-carbazol-3-yl)(naphthalen- |
| | | 1-yl)methanone |
| 319 | STS-135 | 1-(5-fluoropentyl)-N-tricyclo[3.3.1.13,7] dec-1-yl- |
| | | 1H-indole-3-carboxamide |
| 320 | AM630 | [6-iodo-2-methyl-1-[2-(4-morpholinyl)ethyl]-1H- |
| | | indol-3-yl](4-methoxyphenyl)-methanone |
| 321 | 5-fluoro-3,5-AB- | (S)-N-(1-amino-3-methyl-1-oxobutan-2-yl)-1-(5- |
| | PFUPPYCA | fluoropentyl)-3-(4-fluorophenyl)-1H-pyrazole-5- |
| | | carboxamide |
| 322 | CUMYL-THPINACA | N-(1-methyl-1-phenylethyl)-1-[(tetrahydro-2H- |
| | | pyran-4-yl)methyl]-1H-indazole-3-carboxamide |
| 323 | AB-BICA | N-[(1S)-1-(aminocarbonyl)-2-methylpropyl]-1- |
| | | (phenylmethyl)-1H-indole-3-carboxamide |
| 324 | 5-fluoro BEPIRAPIM | (4-benzylpiperazin-1-yl)(1-(5-fluoropentyl)-1H- |
| | (hydrochloride) | indol-3-yl)methanone, monohydrochloride |
| 325 | JWH 073 2′-naphthyl | (1-butyl-1H-indol-3-yl)(naphthalen-2-yl)- |
| | isomer | methanone |
| 326 | JWH 149 | (4-methyl-1-naphthalenyl)(2-methyl-1-pentyl-1H- |
| | | indol-3-yl)-methanone |
| 327 | BB-22 6-HO- | isoquinolin-6-yl 1-(cyclohexylmethyl)-1H-indole- |
| | isoquinoline isomer | 3-carboxylate |
| 328 | MMB018 | N-[(1-pentyl-1H-indol-3-yl)carbonyl]-L-valine, |
| | | methyl ester |
| 329 | NNEI 2′-naphthyl | N-1-naphthalenyl-1-pentyl-1H-indole-3- |
| | isomer | carboxamide |
| 330 | FDU-NNEI | 1-(4-fluorobenzyl)-N-(naphthalen-1-yl)- 1H- |
| | | indole-3-carboxamide |
| 331 | CB-25 | N-cyclopropyl-11-(3-hydroxy-5-pentylphenoxy)- |
| | | undecanamide |
| 332 | 3-CAF | naphthalen-2-yl 1-(2-fluorophenyl)-1H-indazole- |
| | | 3-carboxylate |
| 333 | 5-chloro AKB48 | N-((3s,5s,7s)-adamantan-1-yl)-1-(5-chloropentyl)- |
| | | 1H-indazole-3-carboxamide |
| 334 | JWH 122 N-(4- | (4-methylnaphthalen-1-yl)(1-(pent-4-en-1-yl)-1H- |
| | pentenyl) analog | indol-3-yl)methanone |
| 335 | AB-PINACA N-(3- | N-(1-amino-3-methyl-1-oxobutan-2-yl)-1-(3 - |
| | fluoropentyl) isomer | fluoropentyl)-1H-indazole-3-carboxamide |
| 336 | JWH 073 N-(1,1- | (1-(tert-butyl)-1H-indol-3-yl)(naphthalen-1- |
| | dimethylethyl) isomer | yl)methanone |
| 337 | 5-fluoro NNEI 2′- | 1-(5-fluoropentyl)-N-(naphthalen-2-yl)-1H-indole- |
| | naphthyl isomer | 3-carboxamide |
| 338 | 3-fluoro NNEI | 1-(3-fluoropentyl)-N-(naphthalen-1-yl)-1H-indole- |
| | | 3-carboxamide |
| 339 | 4-fluoro NNEI | 1-(4-fluoropentyl)-N-(naphthalen-1-yl)-1H-indole- |
| | | 3-carboxamide |
| 340 | 5-fluoro PB-22 7-HO- | isoquinolin-7-yl 1-(5-fluoropentyl)-1H-indole-3- |
| | isoquinoline isomer | carboxylate |
| 341 | JWH 019 N-(6- | (1-(6-fluorohexyl)-1H-indol-3-yl)(naphthalen-1- |
| | fluorohexyl) isomer | yl)methanone |
| 342 | JWH 018 2′-naphthyl- | (1-(3-methylbutan-2-yl)-1H-indol-3- |
| | N-(1,2-dimethylbutyl) | yl)(naphthalen-2-yl)methanone |
| | isomer | |
| 343 | AB-CHMINACA 2′- | (S)-N-(1-amino-3-methyl-1-oxobutan-2-yl)-2- |
| | indazole isomer | (cyclohexylmethyl)-2H-indazole-3-carboxamide |
| 344 | 5,3-AB- | (S)-N-(1-amino-3-methyl-1-oxobutan-2-yl)-1- |
| | CHMFUPPYCA | (cyclohexylmethyl)-5-(4-fluorophenyl)-1H- |
| | | pyrazole-3-carboxamide |
| 345 | PF-03550096 | N-[(1S)-1-(aminocarbonyl)-2,2-dimethylpropyl]- |
| | | 2,3-dihydro-3-(3-hydroxy-3-methylbutyl)-2-oxo- |
| | | 1H-benzimidazole-1-carboxamide |
| 346 | JWH 081 5-CH₃O- | (5-methoxynaphthalen-1-yl)(1-pentyl-1H-indol-3- |
| | naphthyl isomer | yl)methanone |
| 347 | 4-MeOPP | 1-(4-Methoxyphenyl) piperazine (hydrochloride) |
| 348 | 3,4-MDMA methylene | N,β-dimethyl-1,3-benzodioxole-5-propanamine, |
| | homolog | monohydrochloride |
| | (hydrochloride) | |
| 349 | NNEI 2′-naphthyl | N-2-naphthalenyl-1-pentyl-1H-indole-3- |
| | isomer | carboxamide |
| 350 | JWH 073 2′-naphthyl- | (1-(sec-butyl)-1H-indol-3-yl)(naphthalen-2-yl)- |
| | N-(1-methylpropyl) | methanone |
| | isomer | |
| 351 | CUMYL-PICA | N-(1-methyl-1-phenylethyl)-1-pentyl-1H-indole-3- |
| | | carboxamide |
| 352 | ATHPINACA isomer 2 | N-(adamantan-2-yl)-1-((tetrahydro-2H-pyran-4- |
| | | yl)methyl)-1H-indazole-3-carboxamide |
| 353 | (+/−)-CP 47,497 | rel-5-(1,1-dimethylheptyl)-2-[(1R,3S)-3- |
| | | hydroxycyclohexyl]-phenol |
| 354 | MMB2201 | N-[[1-(5-fluoropentyl)-1H-indol-3-yl]carbonyl]-L- |
| | | valine, methyl ester |
| 355 | 5-fluoro-2-ADB- | N-(1-amino-3S-methyl-1-oxopentan-2S-yl)-2-(5- |
| | PINACA isomer 2 | fluoropentyl)-2H-indazole-3-carboxamide |
| 356 | 5-fluoro CUMYL- | 1-(5-fluoropentyl)-N-(1-methyl-1-phenylethyl)- |
| | PICA | 1H-indole-3-carboxamide |
| 357 | 5-fluoro CYPPICA | N-(cyclopropylmethyl)-1-(5-fluoropentyl)- 1H- |
| | | indole-3-carboxamide |
| 358 | 5-fluoro APINAC | (3s,5s,7s)-adamantan-1-yl 1-(5-fluoropentyl)-1H- |
| | | indazole-3-carboxylate |
| 359 | MAM2201 N-(2- | (1-(2-fluoropentyl)-1H-indol-3-yl)(4- |
| | fluoropentyl) isomer | methylnaphthalen-1-yl)methanone |
| 360 | HU-211 | 3-(1,1-dimethylheptyl)-6aS,7,10,10aS-tetrahydro- |
| | | 1-hydroxy-6,6-dimethyl-6H-dibenzo[b,d]pyran-9- |
| | | methanol |
| 361 | ATHPINACA isomer 1 | 1-[(tetrahydro-2H-pyran-4-yl)methyl]-N- |
| | | tricyclo[3.3.1.1^{3,7}]dec-1-yl-1H-indazole-3- |
| | | carboxamide |
| 362 | JWH 018 2′-naphthyl- | naphthalen-2-yl(1-(pentan-2-yl)-1H-indol-3- |
| | N-(1-methylbutyl) | yl)methanone |
| | isomer | |
| 363 | MCHB-1 | 1-(cyclohexylmethyl)-2-[(4- |
| | | ethoxyphenyl)methyl]-N,N-diethyl-1H- |
| | | benzimidazole-5-carboxamide |
| 364 | SER-601 | 1,4-dihydro-6-(1-methylethyl)-4-oxo-1-pentyl-N- |
| | | tricyclo[3.3.1.1^{3, 7}]dec-1-yl-3- |
| | | quinolinecarboxamide |
| 365 | (+)-CP 55,940 | 2-((1S,2S,5S)-5-hydroxy-2-(3- |
| | | hydroxypropyl)cyclohexyl)-5-(2-methyloctan-2- |
| | | yl)phenol |
| 366 | PTI-1 (hydrochloride) | N,N-diethyl-2-(1-pentyl-1H-indol-3-yl-4- |
| | | thiazolemethanamine |
| 367 | KM 233 | 6aR,7,10,10aR-tetrahydro-6,6,9-trimethyl-3-(1- |
| | | methyl-1-phenylethyl)-6H-dibenzo[b,d]pyran-1-ol |
| 368 | AB-005 | [1-[(1-methyl-2-piperidinyl)methyl]-1H-indol-3- |
| | | yl](2,2,3,3-tetramethylcyclopropyl)-methanone |
| 369 | PTI-2 (hydrochloride) | N-(2-methoxyethyl)-N-(1-methylethyl)-2-(1- |
| | | pentyl-1H-indol-3-yl)-4-thiazolemethanamine, |
| | | monohydrochloride |
| 370 | JWH 080 | (1-butyl-1H-indol-3-yl)(4-methoxy-1- |
| | | naphthalenyl)-methanone |
| 371 | (−)-CP 47,497 | 2[(1S,3R)-3-hydroxycyclohexyl]-5-(2- |
| | | methyloctan-2-yl)phenol |
| 372 | JWH 098 | (4-methoxy-1-naphthalenyl)(2-methyl-1-pentyl- |
| | | 1H-indol-3-yl)-methanone |
| 373 | 5-fluoro PCN | 1-(5-fluoropentyl)-N-(naphthalen-1-yl)-1H- |
| | | pyrrolo[3,2-c]pyridine-3-carboxamide |
| 374 | CP 47,497-C₉-homolog | cis-5-(1,1-dimethylnonyl)-2-(3- |
| | | hydroxycyclohexyl)-phenol |
| 375 | (+/−)5-epi CP 55,940 | rel-2-((1R,2R,5S)-5-hydroxy-2-(3- |
| | | hydroxypropyl)cyclohexyl)-5-(2-methyloctan-2- |
| | | yl)phenol |
| 376 | (+/−)-WIN 55,212 | [2,3-dihydro-5-methyl-3-(4- |
| | (mesylate) | morpholinylmethyl)pyrrolo[1,2,3-de]-1,4- |
| | | benzoxazin-6-yl]-1-naphthalenyl-methanone, |
| | | methanesulfonate |
| 377 | HU-331 | 3-hydroxy-2-[(1R,6R)-3-methyl-6-(1- |
| | | methylethenyl)-2-cyclohexen-1-yl]-5-pentyl-2,5- |
| | | cyclohexadiene-1,4-dione |
| 378 | HU-210 | 3-(1,1′-dimethylheptyl)-6aR,7,10,10aR-tetrahydro- |
| | | 1-hydroxy-6,6-dimethyl-6H-dibenzo[b,d]pyran-9- |
| | | methanol |
| 379 | (+)-WIN 55,212-2 | [(3R)-2,3-dihydro-5-methyl-3-(4- |
| | (mesylate) | morpholinylmethyl)pyrrolo[1,2,3-de]-1,4- |
| | | benzoxazin-6-yl]-1-naphthalenyl-methanone, |
| | | methanesulfonate |
| 380 | (+/−)CP 55,940 | rel-5-(1,1-dimethylheptyl)-2-[(1R,2R,5R)-5- |
| | | hydroxy-2-(3-hydroxypropyl)cyclohexyl]-phenol |
| 381 | Cannabichromenevarin | 2-methyl-2-(4-methylpent-3-enyl)-7- |
| | | propylchromen-5-ol |
| 382 | CBT, Cannabicitran | (6aα,9β,10aα)-6a,7,8,9,10,10a-hexahydro-6,6,9- |
| | | trimethyl-3-pentyl-1,9-epoxy-6H- |
| | | dibenzo[b,d]pyran |
| 383 | Δ⁸-THC, Δ⁸- | 6aR,7,10,10aR-tetrahydro-6,6,9-trimethyl-3- |
| | Tetrahydrocannabinol | pentyl-6H-dibenzo[b,d]pyran-1-ol |
| 384 | Δ⁹-THC, Δ⁹- | 6aR,7,8,10aR-tetrahydro-6,6,9-trimethyl-3-pentyl- |
| | Tetrahydrocannabinol | 6H-dibenzo[b,d]pyran-1-ol |
| 385 | CBV, Cannabivarin | 6,6,9-trimethyl-3-propyl-6H-dibenzo[b,d]pyran-1- |
| | | ol |
| 386 | CBDV, Cannabidivarin | 2-[(1R,6R)-3-methyl-6-(1-methylethenyl)-2- |
| | | cyclohexen-1-yl]-5-propyl-1,3-benzenediol |
| 387 | CBN Monomethyl | 1-methoxy-6,6,9-trimethyl-3-pentyl-6H- |
| | Ether | dibenzo[b,d]pyran |
| 388 | Cannabigerorcin | 2-[(2E)-3,7-dimethyl-2,6-octadien-1-yl]-5-methyl- |
| | | 1,3-benzenediol |
| 389 | Cannabigerorcinic acid | 3-[(2E)-3,7-dimethyl-2,6-octadien-1-yl]-2,4- |
| | | dihydroxy-6-methyl-benzoic acid |
| 390 | CBL, Cannabicyclol | (1aS,1a¹R,3aR,8bR)-1,1,3a-trimethyl-6-pentyl- |
| | | 1a,1a¹,2,3,3a,8b-hexahydro-1H-4- |
| | | oxabenzo[f]cyclobuta[cd]inden-8-ol |

In one aspect, the invention provides a method wherein the cannabinoid compound or derivative thereof is a compound of table 2.

TABLE 2

| Example | Compound | Molecular Weight | Structure/Name |
|---|---|---|---|
| 1 | Cannabigerol | 316.485 | 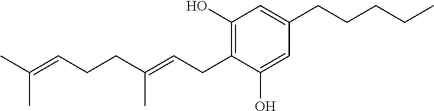 [SMILES: CCCCCc1cc(O)c(C/C=C(\C)CCC=C(C)C)c(O)c1] |
| 2 | Cannabidiol | 314.469 | 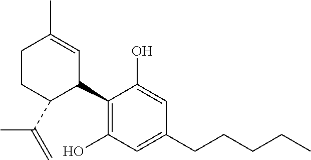 |
| 3 | Cannabichromene | 314.469 | 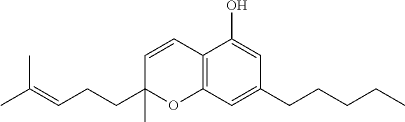 [SMILES: CCCCCc1cc(O)c2c(c1)OC(C)(CCC=C(C)C)C=C2] |
| 4 | Cannabinol | 310.437 | 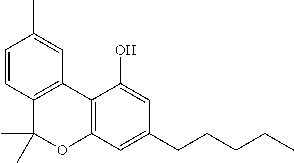 [SMILES: CCCCCc1cc(O)c2c(c1)OC(C)(C)c1ccc(C)cc1-2] |
| 5 | Tetrahydrocannabivarin | 286.415 | 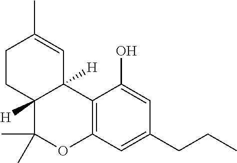 [SMILES: CCCc1cc(O)c2c(c1)OC(C)(C)[C@@H]1CCC(C)=C[C@@H]21] |

Numerous other embodiments of the compound of Formula I-Ig are set forth herein. It is understood that any of the foregoing disclosed compounds may be used in any and all aspects or embodiments of the invention described herein.

Treatment in accordance with the present invention may be symptomatic or prophylactic.

Cannabinoids have demonstrated biological and pharmacological effects, including pain reduction, inhibition of nausea, appetite induction, anxiety and depression reduction, among others (Robson P, Handbook Exp Pharmacol 168:719-756, 2005). Some of these activities are of benefit in cancer therapy, especially for reducing nausea, pain, and depression, and also for increasing appetite (Schrot and Hubbard, Ann. Med. 48:128-141, 2016).

While there are some reports of direct cytotoxic effect of cannabinoids on tumor cells (see, for example, Velasco et al., Nat. Rev. Cancer 12:436-444, 2012), there are no publications that demonstrate MHC induction by cannabinoids. An assay has been developed to indirectly screen for compounds that increase MHC class I expression in a metastatic mouse tumor cell line (U.S. patent Ser. No. 14/548,726, Jefferies et. al., published 28 May 2015). This assay identified numerous compounds that increased MHC expression. Additional screening based on structure-activity profiles, including substructural analysis of cannabinoids, enabled the identification of cannabigerol as a compound with significant activity in the assay. Further experiments demonstrated that cannabigerol increases MHC class I expression in a human tumor cell line. In addition, some other cannabinoids induce expression of MHC class I on tumor cells.

The process of the invention is of general application to mammalian cells, including malignant cells, virally infected cells, and bacterially infected cells. Pharmaceutical compositions of cannabinoids or cannabinoid derivatives may enable medical treatments by enhancing the immunogenicity of tumor cells or pathogen-infected cells in a patient's body, thereby rendering them more susceptible to recognition and elimination by the body's immune system. Such pharmaceutical compositions can be delivered by routes including intravenous, intramuscular, intraperitoneal, oral (including sublingual), intranasal, aerosol (for intrapulmonary administration), parenteral, intra-tumoral, and also ex vivo by treating tumors or dendritic cells with said compound and then reintroducing them into the patient.

Combining such pharmaceutical compositions with other therapeutics, particularly those that activate the immune response such as checkpoint inhibitors, co-activating receptor agonists, and cancer or pathogen-focused vaccines may further increase the potency of the cannabinoid and/or co-therapeutic agents. Appropriate doses are determined by the condition of the patient and degree of severity of the disorder under treatment, but are within the ordinary skill of the attending clinician based upon analogy with other malignancy or infectious disease treating pharmaceuticals.

When administered as a combination, the therapeutic agents can be formulated as separate compositions that are administered at the same time or sequentially at different times, or the therapeutic agents can be given as a single composition. The phrase “co-therapy” (or “combination-therapy”), in defining use of a compound of the present invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent. Specifically, the administration of compounds of the present invention may be in conjunction with additional therapies known to those skilled in the art.

If formulated as a fixed dose, such combination products employ the compounds of this invention within the accepted dosage ranges. Compounds of any of the embodiments described herein may also be administered sequentially with known agents for use in treating cancer, or with antiviral or antimicrobial therapeutic agents, when a combination formulation is inappropriate. The invention is not limited in the sequence of administration as compounds of the invention may be administered either prior to, simultaneous with, or after administration of a known therapeutic agent.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound. The compounds of the present disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

The term “comprising” is meant to be open ended, i.e., all encompassing and non-limiting. It may be used herein synonymously with “having” or “including”. Comprising is intended to include each and every indicated or recited component or element(s) while not excluding any other components or elements. For example, if a composition is said to comprise A and B, this means that the composition has A and B in it, but may also include C or even C, D, E, and other additional components.

Certain compounds of the invention may possess asymmetric carbon atoms (optical centers) or double bonds; the racemates, enantiomers, diastereomers, geometric isomers and individual isomers are all intended to be encompassed within the scope of the invention. Furthermore, atropisomers and mixtures thereof such as those resulting from restricted rotation about two aromatic or heteroaromatic rings bonded to one another are intended to be encompassed within the scope of the invention. For example, when R4 is a phenyl group and is substituted with two groups bonded to the C atoms adjacent to the point of attachment to the N atom of the triazole, then rotation of the phenyl may be restricted. In some instances, the barrier of rotation is high enough that the different atropisomers may be separated and isolated.

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the mirror image enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. If the stereochemistry of a structure or a portion of a structure is not indicated with, for example, bold or dashed lines, the structure or portion of the structure is to be interpreted as encompassing all stereoisomers of it. A bond drawn with a wavy line indicates that both stereoisomers are encompassed. This is not to be confused with a wavy line drawn perpendicular to a bond which indicates the point of attachment of a group to the rest of the molecule.

As described above, this invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al. (1997) Tetrahedron 33:2725; Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

As known by those skilled in the art, certain compounds of the invention may exist in one or more tautomeric forms. Because one chemical structure may only be used to represent one tautomeric form, it will be understood that for convenience, referral to a compound of a given structural formula includes tautomers of the structure represented by the structural formula.

Compounds of the invention may be depicted structurally and named as compounds in one form. However, it is specifically contemplated and known that the compounds exist in other forms and thus compounds in all tautomeric forms are expressly considered to be part of the invention. Depending on the compound, some compounds may exist primarily in one form more than another.

Compounds of the present disclosure include, but are not limited to, compounds of Formula I-Ig and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. As used herein, the term “compound” encompasses not only the compound itself, but also a pharmaceutically acceptable salt thereof, a solvate thereof, a chelate thereof, a non-covalent complex thereof, a prodrug thereof, and mixtures of any of the foregoing. In some embodiments, the term “compound” encompasses the compound itself, pharmaceutically acceptable salts thereof, tautomers of the compound, pharmaceutically acceptable salts of the tautomers, and ester prodrugs such as (C₁-C₄)alkyl esters. In other embodiments, the term “compound” encompasses the compound itself, pharmaceutically acceptable salts thereof, tautomers of the compound, pharmaceutically acceptable salts of the tautomers.

The term “solvate” refers to the compound formed by the interaction of a solvent and a compound. Suitable solvates are pharmaceutically acceptable solvates, such as hydrates, including monohydrates and hemi-hydrates.

Disease” refers to any disease, disorder, condition, symptom, or indication.

The term “cannabinoid compound” means a compound that can be isolated from cannabis, also known as “phytocannabinoids,” or a synthetic compound that also stimulates or inhibits cannabinoid receptors CB-1 and/or CB-2. Such synthetic compounds can also be described as cannabimimetics. Synthetic cannabinoids include the indoles described in U.S. Pat. No. 6,900,236, or U.S. Pat. No. 6,013,648.

Phytocannabinoids include tetrahydrocannabinol (THC), tetrahydrocannabinolic acid (THCA), cannabidiol (CBD), cannabidiolic acid (CBDA), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin(CBCV), cannabigerovarin (CBGV), cannabigerol monomethyl ether (CBGM), cannabielsoin (CBE), and cannabicitran (CBT).

The term “derivative” used herein shall include any conventionally known derivatives of the cannabinoid, such as, inter alia, solvates. It may be convenient or desirable to prepare, purify, and/or handle a corresponding solvate of the compound described herein, which may be used in any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc., depending on the number of water molecules present per molecule of substrate. The term derivative shall especially include a salt. Suitable salts of the cannabinoid are well known and are described in the prior art. Salts of organic and inorganic acids and bases may be used to make pharmaceutically acceptable salts. Such acids include, without limitation, hydrofluoric, hydrochloric, hydrobromic, hydroiodic, sulfuric, nitric, phosphoric, citric, succinic, maleic, and palmitic acids. The bases include such compounds as sodium and ammonium hydroxides. Those skilled in the art are familiar with quaternizing agents that can be used to make pharmaceutically acceptable quaternary ammonium derivatives of the cannabinoid. These include without limitation methyl and ethyl iodides and sulphates.

“Alkyl” refers to a saturated branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl and propan-2-yl, butyls such as butan-1-yl, butan-2-yl, 2-methyl-propan-1-yl, 2-methyl-propan-2-yl, tert-butyl, and the like. In certain embodiments, an alkyl group comprises 1 to 20 carbon atoms. In some embodiments, alkyl groups include 1 to 8 carbon atoms or 1 to 6 carbon atoms whereas in other embodiments, alkyl groups include 1 to 4 carbon atoms. In still other embodiments, an alkyl group includes 1 or 2 carbon atoms. Branched chain alkyl groups include at least 3 carbon atoms and typically include 3 to 7, or in some embodiments, 3 to 6 carbon atoms. An alkyl group having 1 to 6 carbon atoms may be referred to as a (C₁-C₆)alkyl group and an alkyl group having 1 to 4 carbon atoms may be referred to as a (C₁-C₄)alkyl. This nomenclature may also be used for alkyl groups with differing numbers of carbon atoms. The term “alkyl may also be used when an alkyl group is a substituent that is further substituted in which case a bond between a second hydrogen atom and a C atom of the alkyl substituent is replaced with a bond to another atom such as, but not limited to, a halogen, or an O, N, or S atom. For example, a group —O—(C₁-C₆ alkyl)-OH will be recognized as a group where an —O atom is bonded to a C₁-C₆ alkyl group and one of the H atoms bonded to a C atom of the C₁-C₆ alkyl group is replaced with a bond to the O atom of an —OH group. As another example, a group —O—(C₁-C₆ alkyl)-O—(C₁-C₆ alkyl) will be recognized as a group where an —O atom is bonded to a first C₁-C₆ alkyl group and one of the H atoms bonded to a C atom of the first C₁-C₆ alkyl group is replaced with a bond to a second O atom that is bonded to a second C₁-C₆ alkyl group.

“Alkenyl” refers to an unsaturated branched or straight-chain hydrocarbon group having at least one carbon-carbon double bond derived by the removal of one hydrogen atom from a single carbon atom of a parent alkene. The group may be in either the Z- or E-form (cis or trans) about the double bond(s). Typical alkenyl groups include, but are not limited to, ethenyl; propenyls such as prop-1-en-1-yl, prop-1-en-2-yl, prop-2-en-1-yl (allyl), and prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, and buta-1,3-dien-2-yl; and the like. In certain embodiments, an alkenyl group has 2 to 20 carbon atoms and in other embodiments, has 2 to 6 carbon atoms. An alkenyl group having 2 to 6 carbon atoms may be referred to as a (C₂-C₆)alkenyl group.

“Alkoxy” refers to a radical-OR where R represents an alkyl group as defined herein. Representative examples include, but are not limited to, methoxy, ethoxy, propoxy, butoxy, and the like. Typical alkoxy groups include 1 to 10 carbon atoms, 1 to 6 carbon atoms or 1 to 4 carbon atoms in the R group. Alkoxy groups that include 1 to 6 carbon atoms may be designated as —O—(C₁-C₆) alkyl or as —O—(C₁-C₆ alkyl) groups. In some embodiments, an alkoxy group may include 1 to 4 carbon atoms and may be designated as —O—(C₁-C₄) alkyl or as —O—(C₁-C₄ alkyl) groups group.

“Aryl” refers to a monovalent aromatic hydrocarbon group derived by the removal of one hydrogen atom from a single carbon atom of a parent aromatic ring system. Aryl encompasses monocyclic carbocyclic aromatic rings, for example, benzene. Aryl also encompasses bicyclic carbocyclic aromatic ring systems where each of the rings is aromatic, for example, naphthalene. Aryl groups may thus include fused ring systems where each ring is a carbocyclic aromatic ring. In certain embodiments, an aryl group includes 6 to 10 carbon atoms. Such groups may be referred to as C₆-C₁₀ aryl groups. Aryl, however, does not encompass or overlap in any way with heteroaryl as separately defined below. Hence, if one or more carbocyclic aromatic rings is fused with an aromatic ring that includes at least one heteroatom, the resulting ring system is a heteroaryl group, not an aryl group, as defined herein. Said “aryl” group may have 1 to 3 substituents such as lower alkyl, hydroxy, halo, haloalkyl, nitro, cyano, alkoxy and lower alkylamino.

The term “aralkyl” embraces aryl-substituted alkyl radicals. Preferable aralkyl radicals are “lower aralkyl” radicals having aryl radicals attached to alkyl radicals having one to six carbon atoms. Examples of such radicals include benzyl, diphenylmethyl and phenylethyl. The aryl in said aralkyl may be additionally substituted with halo, alkyl, alkoxy, halkoalkyl and haloalkoxy.

The term “aryloxy” embraces aryl radicals, as defined above, attached to an oxygen atom. Examples of such radicals include phenoxy and naphthyloxy.

The term “aryloxycarbonyl” embraces aryloxy radicals, as defined above, attached to carbonyl radical. Examples of such radicals include naphthyloxycarbonyl.

“Carbonyl” refers to the radical-C(O) which may also be referred to as —C(═O) group.

“Cyano” refers to the radical —CN.

The term “cyanoalkyl” embraces linear or branched alkyl radicals having one to about ten carbon atoms any one of which may be substituted with one cyano radicals. More preferred cyanoalkyl radicals are “lower cyanoalkyl” radicals having one to six carbon atoms and one cyano radical. Examples of such radicals include cyanobutyl.

“Cycloalkyl” refers to a saturated cyclic alkyl group derived by the removal of one hydrogen atom from a single carbon atom of a parent cycloalkane. Typical cycloalkyl groups include, but are not limited to, groups derived from cyclopropane, cyclobutane, cyclopentane, cyclohexane, cycloheptane, cyclooctane, and the like. Cycloalkyl groups may be described by the number of carbon atoms in the ring. For example, a cycloalkyl group having 3 to 8 ring members may be referred to as a (C₃-C₈)cycloalkyl, a cycloalkyl group having 3 to 7 ring members may be referred to as a (C₃-C₇)cycloalkyl and a cycloalkyl group having 4 to 7 ring members may be referred to as a (C₄-C₇)cycloalkyl. In certain embodiments, the cycloalkyl group can be a (C₃-C₁₀)cycloalkyl, a (C₃-C₈)cycloalkyl, a (C₃-C₇)cycloalkyl, a (C₃-C₆)cycloalkyl, or a (C₄-C₇)cycloalkyl group and these may be referred to as C₃-C₁₀ cycloalkyl, C₃-C₈ cycloalkyl, C₃-C₇ cycloalkyl, C₃-C₆ cycloalkyl, or C₄-C₇ cycloalkyl groups using alternative language.

The term “cycloalkylalkyl”, embraces radicals having a cycloalkyl radical as defined above, attached to an alkyl radical. Examples of such cycloalkylalkyl radicals includes cyclohexylmethyl.

“Heterocyclyl” refers to a cyclic group that includes at least one saturated, partially unsaturated, but non-aromatic, cyclic ring. Heterocyclyl groups include at least one heteroatom as a ring member. Typical heteroatoms include, O, S and N and are independently chosen. Heterocyclyl groups include monocyclic ring systems and bicyclic ring systems. Bicyclic heterocyclyl groups include at least one non-aromatic ring with at least one heteroatom ring member that may be fused to a cycloalkyl ring or may be fused to an aromatic ring where the aromatic ring may be carbocyclic or may include one or more heteroatoms. The point of attachment of a bicyclic heterocyclyl group may be at the non-aromatic cyclic ring that includes at least one heteroatom or at another ring of the heterocyclyl group. For example, a heterocyclyl group derived by removal of a hydrogen atom from one of the 9 membered heterocyclic compounds shown below may be attached to the rest of the molecule at the 5-membered ring or at the 6-membered ring. In some embodiments, a heterocyclyl group includes 5 to 10 ring members of which 1, 2, 3 or 4 or 1, 2, or 3 are heteroatoms independently selected from O, S, or N. In other embodiments, a heterocyclyl group includes 3 to 7 ring members of which 1, 2, or 3 heteroatom are independently selected from O, S, or N. In such 3-7 membered heterocyclyl groups, only 1 of the ring atoms is a heteroatom when the ring includes only 3 members and includes 1 or 2 heteroatoms when the ring includes 4 members. In some embodiments, a heterocyclyl group includes 3 or 4 ring members of which 1 is a heteroatom selected from O, S, or N. In other embodiments, a heterocyclyl group includes 5 to 7 ring members of which 1, 2, or 3 are heteroatoms independently selected from O, S, or N. Typical heterocyclyl groups include, but are not limited to, groups derived from epoxides, aziridine, azetidine, imidazolidine, morpholine, piperazine, piperidine, hexahydropyrimidine, 1,4,5,6-tetrahydropyrimidine, pyrazolidine, pyrrolidine, quinuclidine, tetrahydrofuran, tetrahydropyran, benzimidazolone, pyridinone, and the like. Heterocyclyl groups may be fully saturated, but may also include one or more double bonds. Examples of such heterocyclyl groups include, but are not limited to, 1,2,3,6-tetrahydropyridinyl, 3,6-dihydro-2H-pyranyl, 3,4-dihydro-2H-pyranyl, 2,5-dihydro-1H-pyrolyl, 2,3-dihydro-1H-pyrolyl, 1H-azirinyl, 1,2-dihydroazetenyl, and the like. Substituted heterocyclyl also includes ring systems substituted with one or more oxo (═O) or oxide (—O—) substituents, such as piperidinyl N-oxide, morpholinyl-N-oxide, 1-oxo-1-thiomorpholinyl, pyridinonyl, benzimidazolonyl, benzo[d]oxazol-2(3H)-only, 3,4-dihydroisoquinolin-1(2H)-only, indolinonly, 1H-imidazo[4,5-c]pyridin-2(3H)-only, 7H-purin-8(9H)-only, imidazolidin-2-only, 1H-imidazol-2(3H)-only, 1,1-dioxo-1-thiomorpholinyl, and the like.

The term “heterocyclylalkyl”, embraces radicals having a heterocyclyl radical as defined above, attached to an alkyl radical. Examples of such heterocyclylalkyl radicals includes piperidinylmethyl.

The term “alkenylcarbonyl” embraces radicals having a carbonyl radical substituted with an alkenyl radical. More preferred alkenylcarbonyl radicals are “lower alkenylcarbonyl” radicals having two to six carbon atoms. Examples of such radicals include ethenylcarbonyl.

The term “arylcarbonyl” embraces radicals having a carbonyl radical substituted with an aryl radical. More preferred arylcarbonyl radicals include phenylcarbonyl.

The term “cycloalkylcarbonyl” embraces radicals having a carbonyl radical substituted with a cycloalklyl radical.

The term “heterocyclylcarbonyl” embraces radicals having a carbonyl radical substituted with a heterocyclyl radical.

The term “arylalkylcarbonyl” embraces radicals having a carbonyl radical substituted with an arylalkyl radical. More preferred arylcarbonyl radicals include benzylcarbonyl.

The term “heterocyclylalkylcarbonyl” embraces radicals having a carbonyl radical substituted with a heterocyclylalkyl radical.

The term “alkoxycarbonyl” means a radical containing an alkoxy radical, as defined above, attached via an oxygen atom to a carbonyl radical. Preferably, “lower alkoxycarbonyl” embraces alkoxy radicals having one to six carbon atoms. Examples of such “lower alkoxycarbonyl” ester radicals include substituted or unsubstituted methoxycarbonyl, ethoxycarbonyl, propoxycarbonyl, butoxycarbonyl and hexyloxycarbonyl.

The term “aminocarbonyl” when used by itself or with other terms such as “aminocarbonylalkyl”, “N-alkylaminocarbonyl”, “N-arylaminocarbonyl”, and “N,N-dialkylaminocarbonyl”, denotes an amide group of the formula —C(═O)NH₂. The terms “N-alkylaminocarbonyl” and “N,N-dialkylaminocarbonyl” denote aminocarbonyl radicals which have been substituted with one alkyl radical and with two alkyl radicals, respectively. More preferred are “lower alkylaminocarbonyl” having lower alkyl radicals as described above attached to an aminocarbonyl radical.

The terms “arylaminocarbonyl” and “N-alkyl-N-arylaminocarbonyl” denote aminocarbonyl radicals substituted, respectively, with one aryl radical, or one alkyl and one aryl radical.

The term aminocarbonylalkylaminocarbonyl denotes alkylaminocarbonyl radicals substituted with one aminocarbonyl radical.

The term alkoxycarbonylalkylaminocarbonyl, denotes alkylaminocarbonyl radicals substituted with one alkoxycarbonyl radical.

The term aminocarbonyl(arylalkyl)aminocarbonyl, denotes arylalkylaminocarbonyl radicals substituted with one aminocarbonyl radical.

The term “N-cycloalkylaminocarbonyl” denoted aminocarbonyl radicals which have been substituted with at least one cycloalkyl radical. More preferred are “lower cycloalkylaminocarbonyl” having lower cycloalkyl radicals of three to seven carbon atoms, attached to an aminocarbonyl radical.

The terms “heterocyclylaminocarbonyl” denote aminocarbonyl radicals substituted with one heterocyclyl radical.

The term “heterocyclylcarbonylalkyl” embraces radicals having an alkyl substituted with a heterocyclylcarbonyl radical.

“Halo” or “halogen” refers to a fluoro, chloro, bromo, or iodo group.

“Haloalkyl” refers to an alkyl group in which at least one hydrogen is replaced with a halogen. Thus, the term “haloalkyl” includes monohaloalkyl (alkyl substituted with one halogen atom) and polyhaloalkyl (alkyl substituted with two or more halogen atoms). Representative “haloalkyl” groups include difluoromethyl, 2,2,2-trifluoroethyl, 2,2,2-trichloroethyl, and the like. The term “perhaloalkyl” means, unless otherwise stated, an alkyl group in which each of the hydrogen atoms is replaced with a halogen atom. For example, the term “perhaloalkyl”, includes, but is not limited to, trifluoromethyl, pentachloroethyl, 5-fluoropentyl, and the like.

“Heteroaryl” refers to a monovalent heteroaromatic group derived by the removal of one hydrogen atom from a single atom of a parent heteroaromatic ring system. Heteroaryl groups typically include 5- to 14-membered, but more typically include 5- to 10-membered aromatic, monocyclic, bicyclic, and tricyclic rings containing one or more, for example, 1, 2, 3, or 4, or in certain embodiments, 1, 2, or 3, heteroatoms chosen from O, S, or N, with the remaining ring atoms being carbon. In monocyclic heteroaryl groups, the single ring is aromatic and includes at least one heteroatom. In some embodiments, a monocyclic heteroaryl group may include 5 or 6 ring members and may include 1, 2, 3, or 4 heteroatoms, 1, 2, or 3 heteroatoms, 1 or 2 heteroatoms, or 1 heteroatom where the heteroatom(s) are independently selected from O, S, or N. In bicyclic aromatic rings, both rings are aromatic. In bicyclic heteroaryl groups, at least one of the rings must include a heteroatom, but it is not necessary that both rings include a heteroatom although it is permitted for them to do so. For example, the term “heteroaryl” includes a 5- to 7-membered heteroaromatic ring fused to a carbocyclic aromatic ring or fused to another heteroaromatic ring. In tricyclic aromatic rings, all three of the rings are aromatic and at least one of the rings includes at least one heteroatom. For fused, bicyclic and tricyclic heteroaryl ring systems where only one of the rings contains one or more heteroatoms, the point of attachment may be at the ring including at least one heteroatom or at a carbocyclic ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Heteroaryl does not encompass or overlap with aryl as defined above. Examples of heteroaryl groups include, but are not limited to, groups derived from acridine, carbazole, cinnoline, furan, imidazole, indazole, indole, indolizine, isobenzofuran, isochromene, isoindole, isoquinoline, isothiazole, 2H-benzo[d][1,2,3]triazole, isoxazole, naphthyridine, oxadiazole, oxazole, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, and the like. In certain embodiments, the heteroaryl group can be between 5 to 20 membered heteroaryl, such as, for example, a 5 to 14 membered or 5 to 10 membered heteroaryl. In certain embodiments, heteroaryl groups can be those derived from thiophene, pyrrole, benzothiophene, 2H-benzo[d][1,2,3]triazole benzofuran, indole, pyridine, quinoline, imidazole, benzimidazole, oxazole, tetrazole, and pyrazine.

The term “heterocyclyloxy” embraces heterocyclyl radicals, as defined above, attached to an oxygen atom. Examples of such radicals include quinolinyloxy.

The term “heterocyclyloxycarbonyl” embraces heterocyclyloxy radicals, as defined above, attached to carbonyl radical. Examples of such radicals include quinolinyloxycarbonyl.

The term “heterocyclylalkyl” embraces heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Examples include such radicals as pyridylmethyl and thienylmethyl.

The term “hydrido” or “H” denotes a single hydrogen atom (H). This hydrido radical may be attached, for example, to an oxygen atom to form a hydroxyl radical or two hydrido radicals may be attached to a carbon atom to form a methylene (—CH₂—) radical.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

“Pharmaceutically acceptable” refers to generally recognized for use in animals, and more particularly in humans.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Stereoisomer” refers to an isomer that differs in the arrangement of the constituent atoms in space. Stereoisomers that are mirror images of each other and optically active are termed “enantiomers,” and stereoisomers that are not mirror images of one another and are optically active are termed “diastereomers.”

“Subject” includes mammals and humans. The terms “human” and “subject” are used interchangeably herein. Similarly, “patient” is also a subject.

“Therapeutically effective amount” refers to the amount of a compound that, when administered to a subject for treating a disease, or at least one of the clinical symptoms of a disease or disorder, is sufficient to affect such treatment for the disease, disorder, or symptom. As those skilled in the art will recognize. this amount is typically not limited to a single dose, but may comprise multiple dosages over a significant period of time as required to bring about a therapeutic or prophylactic response in the subject. Thus, a “therapeutically effective amount” is not limited to the amount in a single capsule or tablet, but may include more than one capsule or tablet, which is the dose prescribed by a qualified physician or medical care provider. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

Reference is made in detail to embodiments of the present disclosure. While certain embodiments of the present disclosure are described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

In some such embodiments, the compound may be any one of these presented above.

In some such embodiments, the embodiment provides any of the compounds shown above or a pharmaceutically acceptable salt thereof.

In still other such embodiments, the embodiment provides any of the compounds shown above, or a pharmaceutically acceptable salt thereof, or a mixture thereof.

In some embodiments, the compound is a salt. Such salts may be anhydrous or associated with water as a hydrate. In some embodiments, the compound may be in a neutral form as a base or an acid.

Also provided are pharmaceutical compositions that include the compound or the pharmaceutically acceptable salt thereof, the tautomer thereof, the pharmaceutically acceptable salt of the tautomer, the stereoisomer of any of the foregoing, or the mixture thereof according to any one of the embodiments and at least one pharmaceutically acceptable excipient, carrier or diluent. In some such embodiments, the compound or the pharmaceutically acceptable salt thereof, the tautomer thereof, the pharmaceutically acceptable salt of the tautomer, the stereoisomer of any of the foregoing, or the mixture thereof according to any one of the embodiments is present in an amount effective for the treatment of an oncology condition or for upregulating MHC-I. In some embodiments, the pharmaceutical composition is formulated for oral delivery whereas in other embodiments, the pharmaceutical composition is formulated for intravenous delivery. In some embodiments, the pharmaceutical composition is formulated for oral administration once a day or QD, and in some such formulations is a unit where the effective amount of the active ingredient ranges from 50 mg to 5000 mg. Alternatively, an oral solution may be provided ranging from a concentration of 1 mg/ml to 50 mg/ml or higher.

One aspect of the invention includes administering a cannabinoid to provide a serum concentration ranging from 0.1 μM to 50 μM. One aspect of the invention includes administering a cannabinoid to provide a serum concentration ranging from 1 μM to 20 μM. One aspect of the invention includes administering a cannabinoid to provide a serum concentration ranging from 5 μM to 20 μM. One aspect of the invention includes administering a cannabinoid to provide a serum concentration of either 10 μM, 20 μM, 5 μM, 1 μM, 15 μM, or 40 μM.

One aspect of the invention includes administering a cannabinoid at a dose of 1 to 100 mg/kg/day, 5-40 mg/kg/day, 10-20 mg/kg/day, 1-2 mg/kg/day, 20-40 mg/kg/day, 45-50 mg/kg/day, 50-60 mg/kg/day, 55-65 mg/kg/day, 60-70 mg/kg/day or 65-75 mg/kg/day.

In some embodiments, the subject is a mammal. In some such embodiments, the mammal is a rodent. In other such embodiments, the mammal is a canine. In still other embodiments, the subject is a primate and, in some such embodiments, is a human.

The pharmaceutical compositions or formulations for the administration of the compounds of this invention may conveniently be presented in unit dosage form and may be prepared by any of the methods well known in the art. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the subject. The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents, such as calcium carbonate, sodium carbonate, lactose, calcium phosphate or sodium phosphate: granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

The compounds may also be administered via edible means.

Formulations for oral use may also be presented as hard gelatin capsules wherein the active ingredient is mixed with an inert solid diluent, for example, calcium carbonate, calcium phosphate, or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil, or coconut oil, or in a mineral oil such as liquid paraffin. The oily suspensions may contain a thickening agent, for example beeswax, hard paraffin, or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The pharmaceutical compositions may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include, for example, cocoa butter and polyethylene glycols.

For topical use, creams, ointments, jellies, solutions, or suspensions, etc., containing the compounds of the invention are employed. As used herein, topical application is also meant to include the use of mouthwashes and gargles.

The compounds of the invention can be administered to provide systemic distribution of the compound within the patient. Therefore, in some embodiments, the compounds of the invention are administered to produce a systemic effect in the body.

As indicated above, the compounds of the invention may be administered via oral, mucosal (including sublingual, buccal, rectal, nasal, or vaginal), parenteral (including subcutaneous, intramuscular, bolus injection, intra-arterial, or intravenous), transdermal, or topical administration. In some embodiments, the compounds of the invention are administered via mucosal (including sublingual, buccal, rectal, nasal, or vaginal), parenteral (including subcutaneous, intramuscular, bolus injection, intra-arterial, or intravenous), transdermal, or topical administration. In other embodiments, the compounds of the invention are administered via oral administration. In still other embodiments, the compounds of the invention are not administered via oral administration.

The compound of the invention, the pharmaceutically acceptable salt thereof, the tautomer thereof, the pharmaceutically acceptable salt of the tautomer, the stereoisomer of any of the foregoing, or the mixture thereof may find use in treating a number of conditions. For example, in some embodiments, the invention comprises methods or uses that include the use or administration of the compound, the pharmaceutically acceptable salt thereof, the tautomer thereof, the pharmaceutically acceptable salt of the tautomer, the stereoisomer of any of the foregoing, or the mixture thereof of the invention, in treating a subject suffering from cancer.

The term “cancer” means a disease in mammals that is characterized by uncontrolled, abnormal cell growth and proliferation. General classes of cancers include carcinomas, lymphomas, sarcomas, and blastemas. A “tumor” or “neoplasm” is an abnormal mass of tissue that results from excessive, uncontrolled, and progressive cell division. Methods described herein are useful for treating cancers and proliferative disorders of any type, including but not limited to, carcinomas, sarcomas, soft tissue sarcomas, lymphomas, hematological cancers (e.g. cancers of the lympoid), leukemias, germ cell tumors, and cancers without solid tumors (e.g., hematopoietic cancers). In various aspects, the compounds can be used to treat cancers and/or tumors originating from and/or effecting any tissue, including but not limited to, lung, breast, epithelium, large bowel, rectum, testicle, bladder, thyroid, gallbladder, bile duct, biliary tract, prostate, colon, stomach, esophagus, pancreas, liver, kidney, uterus, cervix, ovary, and brain tissues. Non-limiting examples of specific cancers treatable with the compounds include, but are not limited to, acute lymphoblastic leukemia, acute myeloid leukemia, chronic lymphocytic leukemia, chronic myelogenous leukemia, adrenocortical carcinoma, AIDS-related lymphoma, anal cancer, astrocytoma, cerebral basal cell carcinoma, bile duct cancer, extrahepatic bladder cancer, bladder cancer, bone cancer, osteosarcoma/malignant fibrous histiocytoma, brain stem glioma, brain tumor, brain stem glioma, cerebral astrocytoma/malignant glioma, ependymoma, medulloblastoma, supratentorial primitive neuroectodermal tumor, visual pathway and hypothalamic glioma, breast cancer, male bronchial adenomas/carcinoids, Burkitt's lymphoma, carcinoid tumor, gastrointestinal carcinoma of unknown primary central nervous system lymphoma, cervical cancer, chronic lymphocytic leukemia, chronic myelogenous leukemia, chronic myeloproliferative disorders, colon cancer, colorectal cancer, cutaneous t-cell lymphoma, mycosis fungoides and sezary syndrome, endometrial cancer, ependymoma, esophageal cancer, Ewing's family tumors, germ cell tumors, extrahepatic bile duct cancer, eye cancer, intraocular melanoma, retinoblastoma, gallbladder cancer, gastric (stomach) cancer, gastrointestinal carcinoid tumors, ovarian gestational, trophoblastic tumors, glioma, hypothalamic skin cancer (melanoma), skin cancer (non-melanoma), skin carcinoma, small cell lung cancer, small intestine cancer, soft tissue sarcoma, squamous cell carcinoma, squamous neck cancer with occult primary, metastatic stomach (gastric) cancer, stomach (gastric) cancer, t-cell lymphoma, testicular cancer, thymoma, thymic carcinoma, thyroid cancer, transitional cell cancer of the renal pelvis, ureter trophoblastic tumors, transitional cell cancer, urethral cancer, uterine cancer, uterine sarcoma, vaginal cancer, hypothalamic glioma, vulvar cancer, Waldenstrom's macroglobulinemia, Wilms' tumor, hairy cell leukemia, head and neck cancer, hepatocellular (liver) cancer, Hodgkin's lymphoma, hypopharyngeal cancer, islet cell carcinoma (endocrine pancreas), Kaposi's sarcoma, kidney (renal cell) cancer, kidney cancer, laryngeal cancer, hairy cell lip and oral cavity cancer, liver cancer, lung cancer, non-small cell lung cancer, small cell lymphoma, Burkitt's lymphoma, cutaneous t-cell, Hodgkin's lymphoma, non-Hodgkin's lymphoma, Waldenstrom's malignant fibrous histiocytoma of bone/osteosarcoma medulloblastoma, intraocular (eye) merkel cell carcinoma, mesothelioma, malignant mesothelioma, metastatic squamous neck cancer with occult primary multiple endocrine neoplasia syndrome, multiple myeloma/plasma cell neoplasm, mycosis fungoides myelodysplastic syndromes, myelodysplastic/myeloproliferative diseases, myelogenous leukemia, multiple myeloproliferative disorders, chronic nasal cavity and paranasal sinus cancer, nasopharyngeal cancer, pleoropulmonary blastoma, osteosarcoma/malignant fibrous histiocytoma of bone, pheochromocytoma, pineoblastoma, and supratentorial primitive neuroectodermal tumors.

In some preferred aspects, the cancer is selected from cancers of the epithelium, colon, brain, breast, kidney, lung, lymphoid and skin.

In some embodiments, the compound of the invention, the pharmaceutically acceptable salt thereof, the tautomer thereof, the pharmaceutically acceptable salt of the tautomer, the stereoisomer of any of the foregoing, or the mixture thereof may find use in treating a number of conditions. For example, in some embodiments, the invention comprises methods or uses that include the use or administration of the compound, the pharmaceutically acceptable salt thereof, the tautomer thereof, the pharmaceutically acceptable salt of the tautomer, the stereoisomer of any of the foregoing, or the mixture thereof of the invention, in treating a subject suffering from bacterial or viral infection.

In some embodiments, the pharmaceutical composition comprises any of the compounds disclosed herein at a purity level suitable for administration to a patient. In some embodiments, the analog has a purity level of at least about 90%, preferably above about 95%, more preferably above about 99%, and a pharmaceutically acceptable diluent, carrier or excipient.

The pharmaceutical compositions may be formulated to achieve a physiologically compatible pH. In some embodiments, the pH of the pharmaceutical composition may be at least 5, or at least 6, or at least 7, depending on the formulation and route of administration.

In various embodiments, single or multiple administrations of the pharmaceutical compositions are administered depending on the dosage and frequency as required and tolerated by the subject. In any event, the composition should provide a sufficient quantity of at least one of the compounds disclosed herein to effectively treat the subject. The dosage can be administered once but may be applied periodically until either a therapeutic result is achieved or until side effects warrant discontinuation of therapy.

The dosing frequency of the administration of the pharmaceutical composition depends on the nature of the therapy and the particular disease being treated. Treatment of a subject with a therapeutically effective amount of a compound, of the invention can include a single treatment or, preferably, can include a series of treatments. In a preferred example, a subject is treated with compound daily, one time per week or biweekly.

Cannabinoids and derivatives thereof are available commercially or can be prepared as described in US patent publications U.S. Pat. Nos. 6,013,648, 4,885,295 or 7,820,144. Alternatively, compounds that interact with and modulate the endogenous cannabinoid receptors, either as agonists, inverse agonists, or antagonists could be prepared as described in Mills et al., Am. J. Med. Sci. 350:59-62, 2015; or De Luca and Fattore, Clin. Thera. 40:1457-1466, 2018.

The invention having been described, the following examples are offered by way of illustration, and not limitation.

### EXAMPLES

### Example 1

### Cannabinoids Stimulate MHC Class I Expression in Mouse and Human Cancer Cells

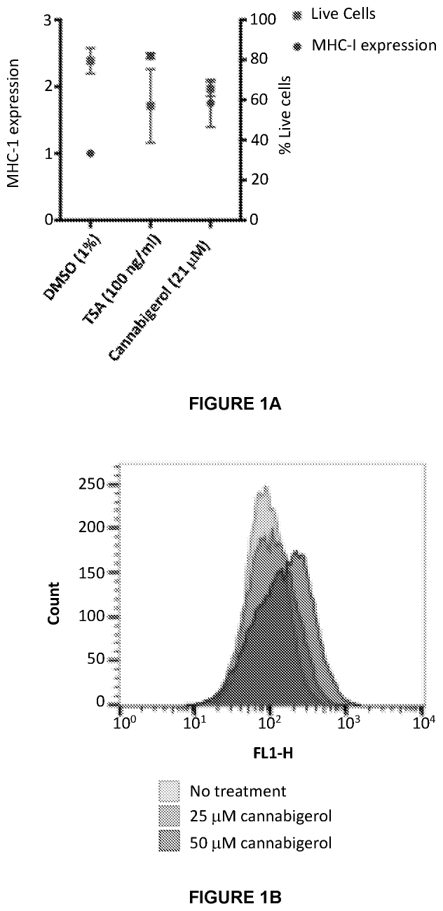

Cannabigerol, a representative cannabinoid, was tested for its ability to also stimulate MHC class I expression in mammalian cancer cell lines. The mouse Lewis lung carcinoma cell line, A9, was treated with 21 μM of cannabigerol for 24 hours prior to flow cytometric analysis of cell surface expression of MHC class I expression levels using the MHC class I antibody W6/32 (Thermo Fisher). Control conditions included the vehicle, dimethyl sulfoxide (DMSO, 1%) alone, and 100 ng/ml of trichostatin A (TSA), a histone deacetylase inhibitor and known inducer of MHC class I expression in mammalian cells. The cells treated with cannabigerol responded with a 1.8-fold increase in MHC class I expression and limited cell death relative to the DMSO control (FIG. 1A). The positive control, TSA, stimulated expression by 2.4-fold.

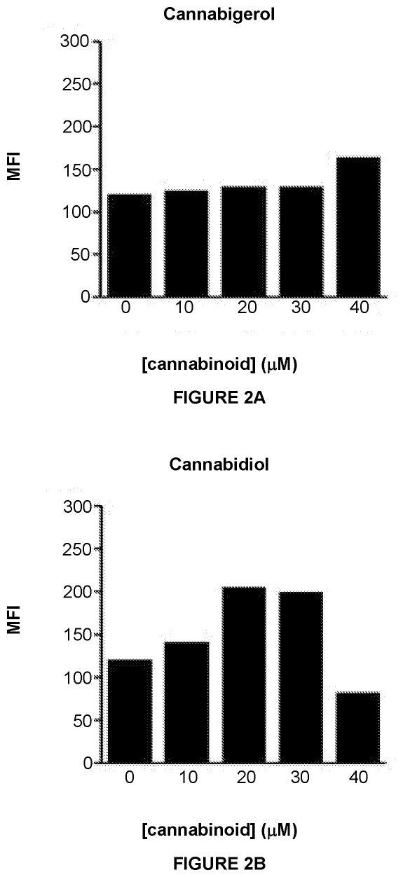

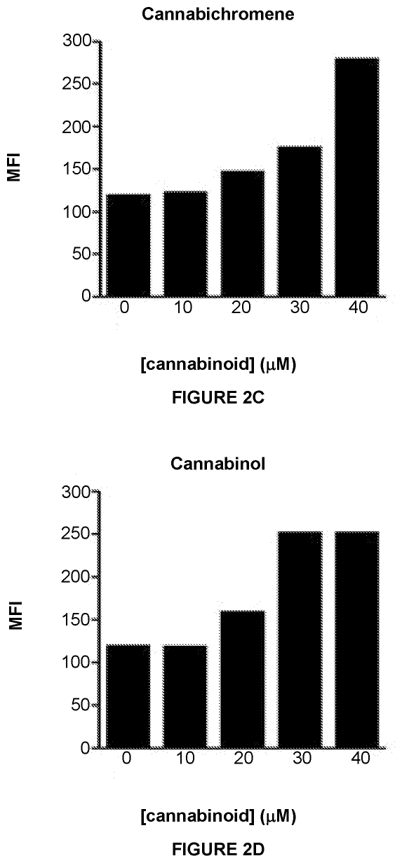

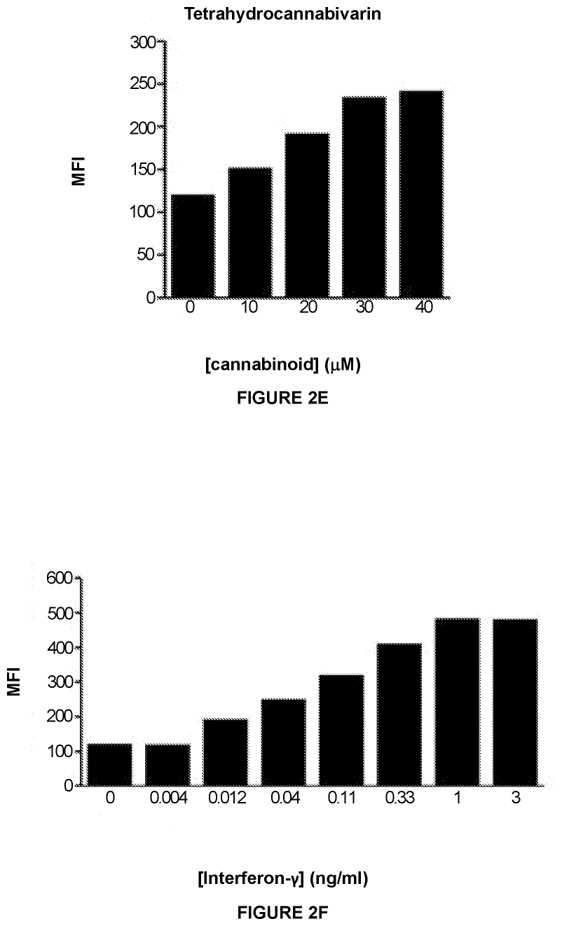

Cannabigerol was also tested for its capacity to induce MHC class I expression in a human colorectal carcinoma cell line, COLO 205. The cells were treated with either 25 μM or 50 μM cannabigerol for 24 hours. Fluorophor (Alexa Fluor 488)-conjugated W6/32 antibody (Thermo Fisher) was used in a flow cytometry assay to quantitate MHC class I expression. At 50 μM, cannabigerol stimulated a 2-fold increase in MHC class I expression relative to non-treated cells, while 25 μM cannabigerol affected a 25% increase in expression (FIG. 1B). Cell viability remained >95% in all treatment conditions. Cannabigerol is only one of many cannabinoid-like molecules with potential biopharmaceutical activity. A small panel of other cannabinoids was tested for MHC class I-inducing capability to assess whether structural modifications may be expected to generate biochemically or pharmacologically improved analogs. COLO 205 cells were treated with up to 40 μM of the cannabinoids 1-5 presented in Tables 1 and 2 and evaluated for cell surface MHC class I expression as described above for cannabigerol. A dose response was observed for all five compounds, with cannabinol, cannabichromene and tetrahydrocannabivarin displaying the greatest potency. At 40 μM, the three compounds induced a ≥2-fold increase in MHC class I expression relative to non-treated COLO 205 cells (Table 3 and FIG. 2A-2E). For reference, interferon-γ, a natural cytokine known to stimulate MHC class I expression in many mammalian cells, was found to induce up to a 4-fold increase in MHC class I expression by the COLO 205 cells (Table 4 and FIG. 2F). Achieving a potency within 2-fold of the maximum induction produced by the natural immune-activating cytokine, interferon-γ, illustrates the great potential of the cannabinoid class of compounds to be optimized for pharmacologically meaningful activity.

TABLE 3
Induction of MHC class I expression on COLO
205 cells by cannabinoids, and magnitude
of response in a flow cytometry assay.

| Example | Concentration (μM) | MFI* | Fold Induction |
|---|---|---|---|
| None | 0 | 121 | 1.00 |
| 1 | 10 | 125 | 1.03 |
| | 20 | 130 | 1.07 |
| | 30 | 130 | 1.07 |
| | 40 | 164 | 1.36 |
| 2 | 10 | 141 | 1.17 |
| | 20 | 205 | 1.69 |
| | 30 | 199 | 1.64 |
| | 40 | 82 | 0.68 |
| 3 | 10 | 120 | 0.99 |
| | 20 | 148 | 1.22 |
| | 30 | 177 | 1.46 |
| | 40 | 280 | 2.31 |
| 4 | 10 | 120 | 0.99 |
| | 20 | 160 | 1.32 |
| | 30 | 253 | 2.09 |
| | 40 | 253 | 2.09 |
| 5 | 10 | 152 | 1.26 |
| | 20 | 192 | 1.59 |
| | 30 | 235 | 1.94 |
| | 40 | 242 | 2.00 |

*Mean Fluorescence Intensity. Flow cytometric signal from COLO 205 cells treated with cannabinoid and stained with MHC class I antibody W6/32 (Thermo Fisher).

TABLE 4
Induction of MHC class I expression on COLO 205 cells by interferon-
γ, and magnitude of response in a flow cytometry assay

| Interferon-γ | | |
|---|---|---|
| Concentration (ng/mL) | MFI | Fold Induction |
| 0 | 121 | 1.00 |
| 0.004 | 119 | 0.98 |
| 0.012 | 193 | 1.60 |
| 0.040 | 250 | 2.07 |
| 0.110 | 321 | 2.65 |
| 0.330 | 410 | 3.39 |
| 1.000 | 483 | 3.99 |
| 3.000 | 482 | 3.98 |

*Mean Fluorescence Intensity. Flow cytometric signal from COLO 205 cells treated with Interferon-γ and stained with MHC class I antibody W6/32 (Thermo Fisher).

### Example 2

### MHC-I Induction COLO 205 Assay

A panel of synthetic cannabinoids (Cayman Chemical, #9002891) was tested for MHC class I-inducing capability in COLO 205 cells as described above. COLO 205 cells were treated with the compounds at a concentration of 35 μM and evaluated for cell surface MHC class I expression after 48 hours. Cells were harvested, stained with MHC-I mAb W6/32 (Thermo Fisher). MHC-I expression was determined by flow cytometry. The results are reported in Table 4. Numerous compounds induced MHC-I expression by the COLO 205 cells, with a total of 53 achieving ≥3-fold induction.

TABLE 4

| | | Induction |
|---|---|---|
| Example | Compound | Fold |
| 6 | (1-((1-methylazepan-3-yl)methyl)-1H-indol-3- | 5.60 |
| | yl)(naphthalen-1-yl)methanone | |
| 7 | (2-iodo-5-nitrophenyl)-(1-(1-methylpiperidin-2-ylmethyl)- | 5.59 |
| | 1H-indol-3-yl)methanone | |
| 8 | ethyl (1-(4-fluorobenzyl)-1H-indazole-3-carbonyl)-L- | 5.50 |
| | valinate | |
| 9 | quinolin-4-yl 1-(cyclohexylmethyl)-1H-indole-3- | 5.41 |
| | carboxylate | |
| 10 | quinolin-4-yl 1-pentyl-1H-indole-3-carboxylate | 5.02 |
| 11 | (S)-N-(1-amino-1-oxohexan-2-yl)-1-pentyl-1H-indazole- | 4.56 |
| | 3-carboxamide | |
| 12 | quinolin-7-yl 1-(5-fluoropentyl)-1H-indole-3-carboxylate | 4.34 |
| 13 | [1-[2-(4-morpholinyl)ethyl]-1H-indol-3-yl]-2- | 4.30 |
| | naphthalenyl-methanone | |
| 14 | N-[[1-(cyclohexylmethyl)-1H-indazol-3-yl]carbonyl]-L- | 4.20 |
| | valine, methyl ester | |
| 15 | N-[[1-[(4-fluorophenyl)methyl]-1H-indazol-3- | 4.20 |
| | yl]carbonyl]-1-valine, methyl ester | |
| 16 | isoquinolin-8-yl 1-(5-fluoropentyl)-1H-indole-3- | 4.19 |
| | carboxylate | |
| 17 | isoquinolin-8-yl 1-pentyl-1H-indole-3-carboxylate | 4.11 |
| 18 | quinolin-7-yl 1-pentyl-1H-indole-3-carboxylate | 4.08 |
| 19 | isoquinolin-4-yl 1-(5-fluoropentyl)-1H-indole-3- | 4.06 |
| | carboxylate | |
| 20 | (2-methoxyphenyl)(1-pentyl-1H-indol-3-yl)-methanone | 3.98 |
| 21 | (4-methoxy-1-naphthalenyl)[1-[2-(4-morpholinyl)ethyl]- | 3.85 |
| | 1H-indol-3-yl]-methanone | |
| 22 | (1-butyl-1H-indol-3-yl)(2-methylnaphthalen-1- | 3.84 |
| | yl)methanone | |
| 23 | (2-iodo-5-nitrophenyl)[1-[[(2R)-1-methyl-2- | 3.64 |
| | piperidinyl]methyl]-1H-indol-3-yl]methanone | |
| 24 | (2-iodophenyl)(1-(1-methylazepan-3-yl)-1H-indol-3- | 3.64 |
| | yl)methanone | |
| 25 | (2-methylnaphthalen-1-yl)(1-pentyl-1H-indol-3- | 3.60 |
| | yl)methanone | |
| 26 | N-((2S,3S)-1-amino-3-methyl-1-oxopentan-2-yl)-1-pentyl- | 3.49 |
| | 1H-indazole-3-carboxamide | |
| 27 | [4-amino-1-[(4-chlorophenyl)methyl]-2-methyl-5-phenyl- | 3.43 |
| | 1H-pyrrol-3-yl]phenyl-methanone | |
| 28 | 2-(3-methoxyphenyl)-1-(1-pentyl-1H-indol-3-yl)-ethanone | 3.42 |
| 29 | N-[(1S)-2-amino-2-oxo-1-(phenylmethyl)ethyl]-1- | 3.41 |
| | (cyclohexylmethyl)-1H-indazole-3-carboxamide | |
| 30 | quinolin-6-yl 1-(5-fluoropentyl)-1H-indole-3-carboxylate | 3.40 |
| 31 | N-[[1-(cyclohexylmethyl)-1H-indol-3-yl]carbonyl]-3- | 3.35 |
| | methyl-L-valine, methyl ester | |
| 32 | [1-[(1-methyl-2-piperidinyl)methyl]-1H-indol-3-yl]-1- | 3.35 |
| | naphthalenyl-methanone | |
| 33 | N-[1-(aminocarbonyl)-2,2-dimethylpropyl]-1- | 3.33 |
| | (cyclohexylmethyl)-1H-indazole-3-carboxamide | |
| 34 | N-(3-Methylbutyl)-3-(1-naphthoyl)-indole | 3.32 |
| 35 | (1-butyl-1H-indol-3-yl)-1-naphthalenyl-methanone | 3.32 |
| 36 | (2-iodophenyl)(1-pentyl-1H-indol-3-yl)-methanone | 3.30 |
| 37 | N-[[1-(cyclohexylmethyl)-1H-indazol-3-yl]carbonyl]-3- | 3.30 |
| | methyl-L-valine, methyl ester | |
| 32 | 1-methoxy-3,3-dimethyl-1-oxobutan-2-yl 1- | 3.30 |
| | (cyclohexylmethyl)-1H-indazole-3-carboxylate | |
| 33 | (S)-N-(1-amino-4-methyl-1-oxopentan-2-yl)-1-pentyl-1H- | 3.30 |
| | indazole-3-carboxamide | |
| 34 | (2-iodophenyl)[1-[(1-methyl-2-piperidinyl)methyl]-1H- | 3.21 |
| | indol-3-yl]-methanone | |
| 35 | (1-(5-bromopentyl)-1H-indol-3-yl)(naphthalen-1- | 3.19 |
| | yl)methanone | |
| 36 | rel-2[(1S,3S)-3-hydroxycyclohexyl)1-5-(2-methyloctan-2- | 3.17 |
| | yl)phenol | |
| 37 | (1-(3-chloropentyl)-1H-indol-3-yl)(2,2,3,3- | 3.17 |
| | tetramethylcyclopropyl)methanone | |
| 38 | 1-(cyclohexylmethyl)-8-quinolinyl ester-1H-indole-3- | 3.15 |
| | carboxylic acid | |
| 39 | 2[(1R,3S)-3-hydroxycyclohexyl]-5-(2-methyloctan-2- | 3.14 |
| | yl)phenol | |
| 40 | (1-(5-fluoropentyl)-1H-benzo[d]imidazol-2- | 3.14 |
| | yl)(naphthalen-1-yl)methanone | |
| 41 | (1-isobutyl-1H-indol-3-yl)(naphthalen-1-yl)methanone | 3.11 |
| 42 | 1-(1-(2-cyclohexylethyl)-1H-indol-3-yl)-2-(3- | 3.07 |
| | methoxyphenyl)ethanone | |
| 43 | 7-methoxy-1-[2-(4-morpholinyl)ethyl]-N-[(1S,2S,4R)- | 3.06 |
| | 1,3,3-trimethylbicyclo[2.2.1]hept-2-yl]-1H-indole-3- | |
| | carboxamide | |
| 44 | 1-(1-(2-cyclohexylethyl)-1H-indol-3-yl)-2-(4- | 3.03 |
| | methoxyphenyl)ethanone | |
| 45 | quinolin-8-yl 1-(3-fluoropentyl)-1H-indole-3-carboxylate | 3.02 |
| 46 | N-[[1-[(4-fluorophenyl)methyl]-1H-indazol-3- | 3.02 |
| | yl]carbonyl]-3-methyl-L-valine, methyl ester | |
| 47 | quinolin-7-yl 1-(cyclohexylmethyl)-1H-indole-3- | 3.01 |
| | carboxylate | |
| 48 | naphthalen-1-yl(1-(pentan-3-yl)-1H-indol-3-yl)methanone | 3.00 |
| 49 | naphthalen-1-yl(1-neopentyl-1H-indol-3-yl)-methanone | 3.00 |
| 50 | (8-chloronaphthalen-1-yl)(1-pentyl-1H-indol-3- | 3.00 |
| | yl)methanone | |
| 51 | (1-(5-fluoropentyl)-1H-indol-3-yl)(2,2,3,3- | 2.99 |
| | tetramethylcyclopropyl)methanone | |
| 52 | isoquinolin-4-yl 1-pentyl-1H-indole-3-carboxylate | 2.98 |
| 53 | isoquinolin-6-yl 1-pentyl-1H-indole-3-carboxylate | 2.97 |
| 54 | (1-(5-fluoropentyl)-1H-indol-3-yl](2-iodophenyl)- | 2.93 |
| | methanone | |
| 55 | 3-(1-naphthalenylcarbonyl)-1H-Indole-1-pentanenitrile | 2.92 |
| 56 | (3-methoxyphenyl)(1-pentyl-1H-indol-3-yl)methanone | 2.90 |
| 57 | [1-[2-(4-morpholinyl)ethyl]-1H-indol-3-yl]-1- | 2.89 |
| | naphthalenyl-methanone | |
| 58 | N-(2-methylbutyl)-3-(1-naphthoyl)-indole | 2.86 |
| 59 | 2-(4-methoxyphenyl)-1-(1-pentyl-1H-indol-3-yl)-ethanone | 2.85 |
| 60 | [5-(2-methylphenyl)-1-pentyl-1H-pyrrol-3-yl]-1- | 2.85 |
| | naphthalenyl-methanone | |
| 61 | 3′R-hydroxy-4-(2-methyloctan-2-yl)-[1,1′S- | 2.85 |
| | bi(cyclohexane)]-3,6-diene-2,5-dione | |
| 62 | (1-(4-fluorobenzyl)-1H-indol-3-yl)(2,2,3,3- | 2.79 |
| | tetramethylcyclopropyl)methanone | |
| 63 | [5-(2-chlorophenyl)-1-pentyl-1H-pyrrol-3-yl]-1- | 2.77 |
| | naphthalenyl-methanone | |
| 64 | 2-(4-chlorophenyl)-1-(1-pentyl-1H-indol-3-yl)-ethanone | 2.77 |
| 65 | 1-naphthalenyl(1-propyl-1H-indol-3-yl)-methanone | 2.77 |
| 66 | 1-naphthalenyl(1-pentyl-1H-pyrrol-3-yl)-methanone | 2.75 |
| 67 | N-[(1S)-1-(aminocarbonyl)-2-methylpropyl]-1- | 2.75 |
| | (cyclohexylmethyl)-1H-indazole-3-carboxaimde | |
| 68 | [1-[(1-methy1-2-piperidinyl)methyl]-1H-indol-3- | 2.72 |
| | yl]tricyclo[3.3.1.13,7]dec-1-yl-methanone | |
| 69 | (1-butyl-1H-indol-3-yl)(4-methylnaphthalen-1- | 2.71 |
| | yl)methanone | |
| 70 | (1-(3-methylbutan-2-yl)-1H-indol-3-yl)(naphthalen-1- | 2.70 |
| | yl)methanone | |
| 71 | (1-pentyl-1H-indol-3-yl)-1-naphthalenyl-methanone | 2.69 |
| 72 | (1-hexyl-1H-pyrrol-3-yl)-1-naphthalenyl-methanone | 2.68 |
| 73 | 1-naphthalenyl[1-(4-penten-1-yl)-1H-indol-3-yl]- | 2.68 |
| | methanone | |
| 74 | 1-(1-(2-cyclohexylethyl)-1H-indol-3-yl)-2-(2- | 2.65 |
| | methoxyphenyl)ethanone | |
| 75 | (2-chloronaphthalen-1-yl)(1-pentyl-1H-indol-3- | 2.65 |
| | yl)methanone | |
| 76 | N-(1-(aminocarbonyl)-2,2-dimethylpropyl]-1-pentyl-1H- | 2.61 |
| | indazole-3-carboxamide | |
| 77 | 1-naphthalenyl(1-pentyl-5-phenyl-1H-pyrrol-3-yl)- | 2.60 |
| | methanone | |
| 78 | (1-propyl-1H-indol-3-yl)(4-propyl-1-naphthalenyl)- | 2.58 |
| | methanone | |
| 79 | [1-(5-fluoropentyl)-1H-indol-3-yl]-1-naphthalenyl- | 2.57 |
| | methanone | |
| 80 | 1-(1-pentyl-1H-indol-3-yl)-2-phenyl-ethanone | 2.57 |
| 81 | (8-methylnaphthalen-1-yl)(1-pentyl-1H-indol-3- | 2.57 |
| | yl)nethanone | |
| 82 | (1-hexyl-5-phenyl-1H-pyrrol-3-yl)-1-naphthalenyl- | 2.54 |
| | methanone | |
| 83 | quinolin-8-yl 1-(2-fluoropentyl)-1H-indole-3-carboxylate | 2.54 |
| 84 | methyl (2S)-2-(1-(4-fluoropentyl)-1H-indazole-3- | 2.53 |
| | carboxamido)-3,3-dimethylbutanoate | |
| 85 | [5-(2-fluorophenyl)-1-pentyl-1H-pyrrol-3-yl]-1- | 2.52 |
| | naphthalenyl-methanone | |
| 86 | 2-(3-chlorophenyl)-1-(1-pentyl-1H-indol-3-yl)-ethanone | 2.51 |
| 87 | (8-bromonaphthalen-1-yl)(1-pentyl-1H-indol-3- | 2.51 |
| | yl)methanone | |
| 88 | N-(1-amino-3,3-dimethyl-1-oxobutan-2-yl)-1-pentyl-1H- | 2.50 |
| | indole-3-carboxamide | |
| 89 | (2-ethylnaphthalen-1-yl)(1-pentyl-1H-indol-3- | 2.50 |
| | yl)methanone | |
| 90 | (2-methoxy-1-naphthalenyl)(1-pentyl-1H-indol-3-yl)- | 2.50 |
| | methanone | |
| 91 | 1-pentyl-N-(phenylmethyl)-1H-indole-3-carboxamide | 2.48 |
| 92 | quinolin-4-yl 1-(5-fluoropentyl)-1H-indole-3-carboxylate | 2.47 |
| 93 | (3,5,7)-adamantan-1-yl(1-(1-methylazepan-3-yl)-1H- | 2.46 |
| | indol-3-yl)methanone | |
| 94 | (1-pentyl-1H-indol-3-yl)(2,2,3,3-tetramethylcyclopropyl)- | 2.45 |
| | methanone | |
| 95 | [N(Z)]-N-[3-(2-methoxyethyl)-4,5-dimethyl-2(3H)- | 2.44 |
| | thiazolylidene]-2,2,3,3-tetramethyl- | |
| | cyclopropanecarboxamide | |
| 96 | (S)-N-(1-amino-3-methyl-1-oxobutan-2-yl)-1- | 2.44 |
| | (cyclohexylmethyl)-1H-indole-3-carboxamide | |
| 97 | (1-(5-bromopentyl)-1H-indazol-3-yl)(naphthalen-1- | 2.44 |
| | yl)methanone | |
| 98 | (2-methyl-1-pentyl-1H-indol-3-yl)-1-naphthalenyl- | 2.43 |
| | methanone | |
| 99 | isoquinolin-5-yl 1-pentyl-1H-indole-3-carboxylate | 2.43 |
| 100 | (S)-N-(1-amino-3-methyl-1-oxobutan-2-yl)-1-pentyl-1H- | 2.43 |
| | indazole-3-carboxamide | |
| 101 | 2-(2-methylphenyl)-1-(1-pentyl-1H-indol-3-yl)ethanone | 2.42 |
| 102 | quinolin-8-yl 1-(4-fluoropentyl)-1H-indole-3-carboxylate | 2.41 |
| 103 | naphthalen-1-yl(1-(tert-pentyl)-1H-indol-3-yl)methanone | 2.41 |
| 104 | (1-(2-fluoropentyl)-1H-indol-3-yl)(naphthalen-1- | 2.41 |
| | yl)methanone | |
| 105 | isoquinolin-5-yl 1-(5-fluoropentyl)-1H-indole-3- | 2.40 |
| | carboxylate | |
| 106 | 1-(1-pentyl-1H-indol-3-yl)-2-(2-methoxyphenyl)-ethanone | 2.39 |
| 107 | (1-butyl-6-methoxy-1H-indol-3-yl)-1-naphthalenyl- | 2.39 |
| | methanone | |
| 108 | 1-pentyl-N-tricyclo[3.3.1.1^{3,7}]dec-1-yl-1H-indole-3- | 2.38 |
| | carboxamide | |
| 109 | [5-(3-fluorophenyl)-1-pentyl-1H-pyrrol-3-yl]-1- | 2.37 |
| | naphthalenyl-methanone | |
| 110 | (2,2,3,3-tetramethylcydopropyl)[1-(4,4,4-trifluorobutyl)- | 2.37 |
| | 1H-indol-3-yl]-methanone | |
| 111 | 2,2,3,3-tetramethyl-cyclopropanecarboxylic acid (2Z)-2- | 2.37 |
| | [1-(5-fluoropentyl)-1,2-dihydro-2-oxo-3H-indol-3- | |
| | ylidene]hydrazide | |
| 112 | (4-fluoro-1-naphthalenyl)[1-(5-fluoropentyl)-1H-indol-3- | 2.37 |
| | yl]-methanone | |
| 113 | (1-(4-chloropentyl)-1H-indol-3-yl)(2,2,3,3- | 2.36 |
| | tetramethylcyclopropyl)methanone | |
| 114 | (1-(3-chloropentyl)-1H-indol-3-yl)(naphthalen-1- | 2.35 |
| | yl)methanone | |
| 115 | (8-ethylnaphthalen-1-yl)(1-pentyl-1H-indol-3- | 2.35 |
| | yl)methanone | |
| 116 | (1-(5-fluoropentyl)-1H-indol-3-yl)(pyridin-3- | 2.34 |
| | yl)methanone, monohydrochloride | |
| 117 | N-((3s,5s,7s)-adamantan-1-yl)-1-(4-fluorobenzyl)-1H- | 2.34 |
| | indazole-3-carboxamide | |
| 118 | (1-heptyl-1H-indol-3-yl)(2,2,3,3-tetramethylcyclopropyl)- | 2.33 |
| | methanone | |
| 119 | N-[1-(aminocarbonyl)-2,2-dimethylpropyl]-1-(5- | 2.33 |
| | fluoropentyl)-3-(4-fluorophenyl)-1H-pyrazole-5- | |
| | carboxamide | |
| 120 | 2-(2-bromophenyl)-1-(1-pentyl-1H-indol-3-yl)-ethanone | 2.32 |
| 121 | 2-[1-(4-chlorobenzoyl)-5-methoxy-2-methyl-1H-indol-3- | 2.32 |
| | yl]-1-(4-morpholinyl)-ethanone | |
| 122 | (1-butyl-2-methyl-1H-indol-3-yl)-1-naphthalenyl- | 2.31 |
| | methanone | |
| 123 | naphthalen-1-yl(1-pentyl-1H-benzo[d]imidazol-2- | 2.31 |
| | yl)methanone | |
| 124 | isoquinolin-4-yl 1-(cyclohexylmethyl)-1H-indole-3- | 2.30 |
| | carboxylate | |
| 125 | (2Z)-2-(1-hexyl-1,2-dihydro-2-oxo-3H-indol-3- | 2.29 |
| | ylidene)hydrazide, benzoic acid | |
| 126 | (1-(5-fluoropentyl)-2-methyl-1H-indol-3-yl)(2,2,3,3- | 2.29 |
| | tetramethylcyclopropyl)methanone | |
| 127 | (1-(sec-butyl)-1H-indol-3-yl)(naphthalen-1-yl)methanone | 2.29 |
| 128 | (1-(3-fluoropentyl)-1H-indol-3-yl)(naphthalen-1- | 2.28 |
| | yl)methanone | |
| 129 | (4-methoxyphenyl)(1-pentyl-1H-indol-3-yl)methanone | 2.27 |
| 130 | (1-(5-fluoropentyl)-1H-indol-3-yl)(3- | 2.26 |
| | iodophenyl)methanone | |
| 131 | quinolin-5-yl 1-pentyl-1H-indole-3-carboxylate | 2.26 |
| 132 | (E)-3,4,4-trimethyl-1-(1-(2-morpholinoethyl)-1H-indol-3- | 2.25 |
| | yl)pent-2-en-1-one | |
| 133 | [1-(3-fluoropentyl)-1H-indol-3-yl](2,2,3,3- | 2.23 |
| | tetramethylcyclopropyl)-methanone | |
| 134 | [1-(5-fluoropentyl)-1H-indazol-3-yl]-1-naphthalenyl- | 2.23 |
| | methanone | |
| 135 | N-(1-amino-1-oxobutan-2-yl)-1-(4-fluorobenzyl)-N- | 2.23 |
| | methyl-1H-indazole-3-carboxamide | |
| 136 | quinolin-3-yl 1-(5-fluoropentyl)-1H-indole-3-carboxylate | 2.22 |
| 137 | (2-methyl-1-(1-methylhexyl)-1H-indol-3-yl]-1- | 2.22 |
| | naphthalenyl-methanone | |
| 138 | (1-(5-chloropentyl)-1H-indol-3-yl)(2,2,3,3- | 2.21 |
| | tetramethylcyclopropyl)methanone | |
| 139 | (1-(5-fluoropentyl)-1H-indol-3-yl)(naphthalen-2- | 2.19 |
| | yl)methanone | |
| 140 | (1-hexyl-1H-indol-3-yl)-1-naphthalenyl-methanone | 2.19 |
| 141 | 1-pentyl-N-8-quinolinyl-1H-indole-3-carboxamide | 2.19 |
| 142 | (1-(5-chloropentyl)-1H-indazol-3-yl)(naphthalen-1- | 2.19 |
| | yl)methanone | |
| 143 | N-[(1S)-1-(aminocarbonyl)-2-methylpropyl]-1-[(2- | 2.19 |
| | fluorophenyl)methyl]-1H-indazole-3-carboxamide | |
| 144 | 1-(5-fluoropentyl)-N-tricyclo[3.3.1.13,7]dec-1-yl-1H- | 2.17 |
| | indole-3-carboxamide | |
| 145 | (1-(4-fluoropentyl)-1H-indol-3-yl)(4-methylnaphthalen-1- | 2.16 |
| | yl)methanone | |
| 146 | (7-chloronaphthalen-1-yl(1-pentyl-1H-indol-3- | 2.16 |
| | yl)methanone | |
| 147 | [1-(2-(4-morpholinyl)ethyl]-1H-indol-3-yl](2,2,3,3- | 2.14 |
| | tetramethylcyclopropyl)-methanone | |
| 148 | (1-(pent-4-en-1-yl)-1H-indol-3-yl)(2,2,3,3- | 2.14 |
| | tetramethylcyclopropyl)methanone | |
| 149 | 1-naphthalenyl(1-pentyl-1H-indazol-3-yl)-methanone | 2.14 |
| 150 | (4-fluoro-1-naphthalenyl)(1-pentyl-1H-indol-3-yl)- | 2.14 |
| | methanone | |
| 151 | N-(1-amino-2,3-dimethyl-1-oxobutan-2-yl)-1-pentyl-1H- | 2.13 |
| | indazole-3-carboxamide | |
| 152 | 1-naphthalenyl[4-(pentyloxy)-1-naphthalenyl]-methanone | 2.12 |
| 153 | [1-(2-fluoropentyl)-1H-indol-3-yl](2,2,3,3- | 2.11 |
| | tetramethylcyclopropyl)-methanone | |
| 154 | (2-ethyl-1-pentyl-1H-indol-3-yl)-1-naphthalenyl- | 2.11 |
| | methanone | |
| 155 | [1-(5-fluoropentyl)-1H-indol-3-yl](4-methyl-1- | 2.08 |
| | naphthalenyl)-methanone | |
| 156 | naphthalen-1-yl(1-(3-(oxiran-2-yl)propyl)-1H-indol-3- | 2.07 |
| | yl)methanone | |
| 157 | (1-(2-chloropentyl)-1H-indol-3-yl)(2,2,3,3- | 2.07 |
| | tetramethylcyclopropyl)methanone | |
| 158 | quinolin-5-yl 1-(5-fluoropentyl)-1H-indole-3-carboxylate | 2.07 |
| 159 | N-cyclopropyl-8-[3-(1,1-dimethylheptyl)-5- | 2.06 |
| | hydroxyphenoxy]-octanamide | |
| 160 | [1-(5-fluoropentyl)-1H-indol-3-yl]tricyclo[3.3.1.13,7]dec- | 2.06 |
| | 1-yl-methanone | |
| 161 | (7-methyl-1-naphthalenyl)(1-pentyl-1H-indol-3-yl)- | 2.06 |
| | methanone | |
| 162 | isoquinolin-5-yl 1-(cyclohexylmethyl)-1H-indole-3- | 2.04 |
| | carboxylate | |
| 163 | (7-methoxy-1-naphthalenyl)(1-pentyl-1H-indol-3-yl)- | 2.04 |
| | methanone | |
| 164 | (1-pentyl-1H-indol-3-yl)(4-propyl-1-naphthalenyl)- | 2.03 |
| | methanone | |
| 165 | 1-(1-pentyl-1H-indol-3-yl)-2-(m-tolyl)ethanone | 2.02 |
| 166 | 1-(5-fluoropentyl)-N-(quinolin-8-yl)-1H-indazole-3- | 2.01 |
| | carboxamide | |
| 167 | naphthalen-1-yl 1-(5-fluoropentyl)-1H-indazole-3- | 2.01 |
| | carboxylate | |
| 168 | [1-(2,2-dimethylpropyl)-1H-indol-3-yl]-2-naphthalenyl- | 2.01 |
| | methanone | |
| 169 | N-(1-amino-1-oxopentan-2-yl)-1-(4-fluorobenzyl)-1H- | 2.01 |
| | indazole-3-carboxamide | |
| 170 | N-[(1S)-1-aminocarbonyl)-2-methylpropyl]-1-(5- | 2.01 |
| | chloropentyl)-1H-indazole-3-carboxamide | |
| 171 | naphthalen-1-yl 1-pentyl-1H-indazole-3-carboxylate | 1.99 |
| 172 | N-[(1S)-1-(aminocarbonyl)-2-methylpropyl]-1-[(4- | 1.99 |
| | fluorophenyl)methyl]-1H-indazole-3-carboxamide | |
| 173 | (1-(4-fluorohexyl)-1H-indol-3-yl)(naphthalen-1- | 1.98 |
| | yl)methanone | |
| 174 | (1-(3-fluoropentyl)-1H-indol-3-yl)(4-methylnaphthalen-1- | 1.96 |
| | yl)methanone | |
| 175 | (6-methoxy-1-pentyl-1H-indol-3-y1)(naphthalen-1-yl)- | 1.95 |
| | methanone | |
| 176 | (1-(3-fluorohexyl)-1H-indol-3-yl)(naphthalen-1- | 1.95 |
| | yl)methanone | |
| 177 | (1-(5-chloropentyl)-1H-indol-3-yl)(naphthalen-1- | 1.94 |
| | yl)methanone | |
| 178 | naphthalen-1-yl(1-pentyl-1H-pyrrol-2-yl)methanone | 1.94 |
| 179 | N-[[1-(5-fluoropentyl)-1H-indazol-3-yl]carbonyl]-3- | 1.94 |
| | methyl-D-valine, methyl ester | |
| 180 | (4-ethyl-1-naphthalenyl)(1-pentyl-1H-indol-3-yl)- | 1.93 |
| | methanone | |
| 181 | 2-(4-methylphenyl)-1-(1-pentyl-1H-indol-3-yl)-ethanone | 1.93 |
| 182 | 3′-carbamoyl-biphenyl-3-yl-undecynecarbamate | 1.93 |
| 183 | N-[(1-pentyl-1H-indazol-3-yl)carbonyl]-L-valine, methyl | 1.91 |
| | ester | |
| 184 | 1-5-fluoropentyl)-N-1-naphthalenyl-1H-indazole-3- | 1.90 |
| | carboxamide | |
| 185 | (1-(5-fluoropentyl)-1H-indazol-3-yl)(2,2,3,3- | 1.90 |
| | tetramethylcyclopropyl)methanone | |
| 186 | N-(1-amino-3,3-dimethyl-1-oxobutan-2-yl)-1-benzyl-1H- | 1.90 |
| | indole-3-carboxamide | |
| 187 | naphthalen-1-yl(1-(pentan-2-yl)-1H-indol-3-yl)methanone | 1.90 |
| 188 | 3-(1,1-dimethylbutyl)-6aR,7,10,10aR-tetrahydro-6,6,9- | 1.90 |
| | trimethyl-6H-dibenzo[b,d]pyran | |
| 189 | (1-(5-chloropentyl)-1H-indol-3-yl)(4-methylnaphthalen-1- | 1.89 |
| | yl)methanone | |
| 190 | 2-(2-chlorophenyl)-1-(1-pentyl-1H-indol-3-yl)-ethanone | 1.89 |
| 191 | (5-methylnaphthalen-1-yl)(1-pentyl-1H-indol-3- | 1.89 |
| | yl)methanone | |
| 200 | 5-methoxy-3-[(2-methoxyphenyl)methyl]-7-pentyl-2H-1- | 1.88 |
| | benzopyran-2-one | |
| 201 | [1-(1-ethylpropyl)-1H-indol-3-yl]-2-naphthalenyl- | 1.87 |
| | methanone | |
| 202 | (1-hexyl-1H-pyrrol-2-yl)-1-naphthalenyl-methanone | 1.87 |
| 203 | (4-methyl-1-naphthalenyl)(1-[2-(4-morpholinyl)ethyl]-1H- | 1.87 |
| | indol-3-yl]-methanone | |
| 204 | 1-[(4-fluorophenyl)methyl]-1H-indole-3-carboxylic acid, | 1.87 |
| | 8-quinolinyl ester | |
| 205 | (4-bromo-1-naphthalenyl)(1-pentyl-1H-indol-3-yl)- | 1.86 |
| | methanone | |
| 206 | quinolin-5-yl 1-(cyclohexylmethyl)-1H-indole-3- | 1.86 |
| | carboxylate | |
| 207 | (1-(2-fluorohexyl)-1H-indol-3-yl)(naphthalen-1- | 1.85 |
| | yl)methanone | |
| 208 | 1-(5-fluoropentyl)-N-(quinolin-8-yl)-1H-indole-3- | 1.84 |
| | carboxamide | |
| 209 | cis-5-(1,1-dimethylhexyl)-2-(3-hydroxycyclohexyl)- | 1.84 |
| | phenol | |
| 210 | methyl (1-(3-fluoropentyl)-1H-indazole-3-carbonyl)-L- | 1.84 |
| | valinate | |
| 211 | (4-methyl-1-naphthalenyl)(1-pentyl-1H-indol-3-yl)- | 1.83 |
| | methanone | |
| 212 | (1-heptyl-5-phenyl-1H-pyrrol-3-yl)-1-naphthalenyl- | 1.83 |
| | methanone | |
| 213 | N-[(1S)-2-amino-2-oxo-1-(phenylmethyl)ethyl]-1-[(4- | 1.81 |
| | fluorophenyl)methyl]-1H-indazole-3-carboxamide | |
| 214 | 1-naphthalenyl[5-(1-naphthalenyl)-1-pentyl-1H-pyrol-3- | 1.80 |
| | yl]-methanone | |
| 215 | (1-(5-fluoropentyl)-1H-indol-3-yl)(pyrrolidin-1- | 1.80 |
| | yl)methanone | |
| 216 | (4-ethyl-1-naphthalenyl)[1-(5-fluoropentyl)-1H-indol-3- | 1.79 |
| | yl]-methanone | |
| 217 | N-(1-amino-3,3-dimethyl-1-oxobutan-2-yl)-1-benzyl-1H- | 1.79 |
| | indazole-3-carboxamide | |
| 218 | isoquinolin-6-yl 1-(5-fluoropentyl)-1H-indole-3- | 1.78 |
| | carboxylate | |
| 219 | (3-methylnaphthalen-1-yl)(1-pentyl-1H-indol-3- | 1.78 |
| | yl)methanone | |
| 220 | (1-(4-fluorobenzyl)-1H-indol-3-yl)(naphthalen-1- | 1.77 |
| | yl)methanone | |
| 221 | N-[(1S)-1-(aminocarbonyl)-2-methylpropyl]-1-[(3- | 1.77 |
| | fluorophenyl)methyl]-1H-indazole-3-carboxamide | |
| 222 | [1-(5-bromopentyl)-1H-indol-3-yl](2,2,3,3- | 1.76 |
| | tetramethylcyclopropyl)-methanone | |
| 223 | (4-chloro-1-naphthalenyl)(1-pentyl-1H-indol-3-yl)- | 1.76 |
| | methanone | |
| 224 | N-1-naphthalenyl-1-pentyl-1H-indazole-3-carboxamide | 1.75 |
| 225 | N-(5-methylhexyl) analog (1-(5-methylhexyl)-1H-indol- | 1.75 |
| | 3-yl)(2,2,3,3-tetramethylcyclopropyl)methanone | |
| 226 | N-[1,1′-biphenyl]-3-yl-carbamic acid, cyclohexyl ester | 1.74 |
| 227 | (S)-N-(1-amino-1-oxo-3-phenylpropan-2-yl)-1-(5- | 1.72 |
| | fluoropentyl)-1H-indazole-3-carboxamide | |
| 228 | 1-(5-fluoropentyl)-N-(1-methyl-1-phenylethyl)-1H- | 1.72 |
| | indazole-3-carboxamide | |
| 229 | methyl (1-(cyc1ohexylmethyl)-1H-indole-3-carbonyl)-L- | 1.72 |
| | valinate | |
| 230 | (1-(5-fluoropentyl)-1H-indazol-3-yl)(pyrrolidin-1- | 1.72 |
| | yl)methanone | |
| 231 | (7-ethyl-1-naphthalenyl)(1-pentyl-1H-indol-3-yl)- | 1.72 |
| | methanone | |
| 232 | 1-pentyl-N-(quinolin-8-yl)-1H-indazole-3-carboxamide | 1.71 |
| 233 | (1-(5-fluorohexyl)-1H-indol-3-yl)(naphthalen-1- | 1.71 |
| | yl)methanone | |
| 234 | (3-methoxynaphthalen-1-yl)(1-pentyl-1H-indol-3- | 1.71 |
| | yl)methanone | |
| 235 | (1-(4-fluoropentyl)-1H-indol-3-yl)(2,2,3,3- | 1.70 |
| | tetramethylcyclopropyl)methanone | |
| 236 | ethyl (1-(5-fluoropentyl)-1H-indazole-3-carbonyl)-L- | 1.70 |
| | valinate | |
| 237 | N-[(1S)-1-(aminocarbonyl)-2-methylpropyl]-1-[(4- | 1.70 |
| | fluorophenyl)methyl]-1H-indole-3-carboxamide | |
| 238 | (5-chloronaphthalen-1-yl)(1-pentyl-1H-indol-3- | 1.70 |
| | yl)methanone | |
| 239 | methyl (S)-2-(9-(cyclohexylmethyl)-9H-carbazole-3- | 1.69 |
| | carboxamido)-3,3-dimethylbutanoate | |
| 240 | (6-methylnaphthalen-1-yl)(1-pentyl-1H-indol-3- | 1.69 |
| | yl)methanone | |
| 241 | rel-5-(1,1-dimethyloctyl)-2-[(1R,3S)-3- | 1.68 |
| | hydroxycyclohexyl]-phenol | |
| 242 | N-[(1R)-2-hydroxy-1-methylethyl-9Z-octadecenamide | 1.67 |
| 243 | (3s,5s,7s)-adamantan-1-yl 1-pentyl-1H-indazole-3- | 1.67 |
| | carboxylate | |
| 244 | (4-ethyl-1-naphthalenyl)(2-methyl-1-pentyl-1H-indol-3- | 1.66 |
| | yl)-methanone | |
| 245 | (1s,3s)-adamantan-1-yl(1-pentyl-1H-indol-3-yl)methanone | 1.66 |
| 246 | (S)-N-(1-amino-1-oxo-3-phenylpropan-2-yl)-1-pentyl-1H- | 1.65 |
| | indole-3-carboxamide | |
| 247 | N-[1-(aminocarbonyl)-2,2-dimethylpropyl]-1-[(4- | 1.64 |
| | fluorophenyl)methyl]-1H-indazole-3-carboxamide | |
| 248 | 1-[(4-fluoropheny1)methyl]-1H-indole-3-carboxylic acid, | 1.63 |
| | 1-naphthalenyl ester | |
| 249 | naphthalen-1-yl 1-pentyl-1H-indole-3-carboxylate | 1.63 |
| 250 | 7-chloro-1-[2-(diethylamino)ethyl]-5-(2-fluorophenyl)- | 1.63 |
| | 1,3-dihydro-2H-1,4-benzodiazepin-2-one | |
| 251 | (1-isopentyl-1H-indol-3-yl)(naphthalen-2-yl)methanone | 1.62 |
| 252 | 3-(1-naphthalenylmethyl)-1-pentyl-1H-indole | 1.61 |
| 253 | [1-(cyclohexylmethyl)-1H-indol-3-yl](4-methoxy-1- | 1.61 |
| | naphthalenyl)-methanone | |
| 254 | isoquinolin-7-yl 1-(cyclohexylmethyl)-1H-indole-3- | 1.60 |
| | carboxylate | |
| 255 | (5-ethyl-1-naphthalenyl)(1-pentyl-1H-indol-3-yl)- | 1.60 |
| | methanone | |
| 256 | quinolin-6-yl 1-pentyl-1H-indole-3-carboxylate | 1.59 |
| 257 | naphthalen-1-yl 1-(5-fluoropentyl)-1H-indole-3- | 1.59 |
| | carboxylate | |
| 258 | 1-pentyl-1H-indazole-3-carboxylic acid, 8-quinolinyl ester | 1.59 |
| 259 | 2-naphthalenyl(1-pentyl-1H-indol-3-yl)-methanone | 1.58 |
| 260 | [1-[(tetrahydro-2H-pyran-4-yl)methyl]-1H-indol-3- | 1.57 |
| | yl](2,2,3,3-tetramethylcyclopropyl)-methanone | |
| 261 | Quinolin-3-yl 1-(cyclohexylmethyl)-1H-indole-3- | 1.56 |
| | carboxylate | |
| 262 | 5-(1,1-dimethylheptyl)-2-[(1R,2R,5R)-5-hydroxy-2-(3- | 1.56 |
| | hydroxypropyl)cyclohexyl]-phenol | |
| 263 | (3-chloronaphthalen-1-yl)(1-pentyl-1H-indol-3- | 1.56 |
| | yl)methanone, hydrate | |
| 264 | (3-ethyl-1-naphthalenyl)(1-pentyl-1H-indol-3-yl)- | 1.56 |
| | methanone | |
| 265 | (4-methoxy-1-naphthalenyl)(1-pentyl-1H-indol-3-yl)- | 1.55 |
| | methanone | |
| 266 | 2Z-(1,2-dihydro-6-methoxy-2-oxo-1-pentyl-3H-indol-3- | 1.55 |
| | ylidene)hydrazide benzoic acid | |
| 267 | (S)-N-(1-amino-1-oxo-3-phenylpropan-2-yl)-1-(5- | 1.55 |
| | fluoropentyl)-1H-indole-3-carboxamide | |
| 268 | methyl (1-(4-fluoropentyl)-1H-indazole-3-carbonyl)-L- | 1.55 |
| | valinate | |
| 269 | N-[[1-[(4-fluorophenyl)methyl]-1H-indol-3-yl]carbonyl]- | 1.54 |
| | 3-methyl-L-valine, methyl ester | |
| 270 | N-[[1-[(4-fluorophenyl)methyl]-1H-indol-3-yl]carbonyl]- | 1.54 |
| | L-valine, methyl ester | |
| 271 | (1-(2-methylbutyl)-1H-indol-3-yl)(naphthalen-2-yl)- | 1.54 |
| | methanone | |
| 272 | (1-ethyl-1H-indol-3-yl)-1-naphthalenyl-methanone | 1.53 |
| 273 | (4-methylpiperazin-1-yl)(1-pentyl-1H-indol-3- | 1.53 |
| | yl)methanone, monohydrochloride | |
| 274 | isoquinolin-7-yl 1-pentyl-1H-indole-3-carboxylate | 1.52 |
| 275 | (6-ethylnaphthalen-1-yl)(1-pentyl-1H-indol-3- | 1.52 |
| | yl)methanone | |
| 276 | quinolin-8-yl 1-(4-fluorobenzyl)-1H-indazole-3- | 1.51 |
| | carboxylate | |
| 277 | [1-(cyclohexylmethyl)-7-methoxy-1H-indol-3-yl](3,4- | 1.50 |
| | dimethyl-1-piperazinyl)-methanone | |
| 278 | 5-([1,1′-biphenyl]-4-ylmethyl)-N,N-dimethyl-2H- | 1.49 |
| | tetrazole-2-carboxamide | |
| 279 | N-[[1-(5-fluoropentyl)-1H-indazol-3-yl]carbonyl]-L- | 1.49 |
| | valine, methyl ester | |
| 280 | [1-(5-fluoropentyl)-6-nitro-1H-indol-3-yl]-1-naphthalenyl- | 1.49 |
| | methanone | |
| 281 | 1-(4-cyanobutyl)-N-(1-methyl-1-phenylethyl)-1H- | 1.48 |
| | indazole-3-carboxamide | |
| 282 | (4-methoxyphenyl)(1-butyl-1H-indol-3-yl)-methanone | 1.47 |
| 283 | (1-(5-fluoropentyl)-1H-indol-3-yl)(4- | 1.46 |
| | iodophenyl)methanone | |
| 284 | (1-isobutyl-1H-indol-3-yl)(naphthalen-2-yl)methanone | 1.46 |
| 285 | (6-chloronaphthalen-1-yl)(1-pentyl-1H-indol-3- | 1.46 |
| | yl)methanone | |
| 286 | 1-(5-fluoropentyl)-N(naphthalen-1-yl)-1H-indole-3- | 1.45 |
| | carboxamide | |
| 287 | N-(1-amino-2-methyl-1-oxobutan-2-y1)-1-(4- | 1.45 |
| | fluorobenzyl)-1H-indazole-3-carboxamide | |
| 288 | 5-([1,1′-biphenyl]-4-ylmethyl)-N,N-dimethyl-1H- | 1.45 |
| | tetrazole-1-carboxamide | |
| 289 | N-[1-(aminocarbonyl)-2,2-dimethylpropyl]-1-(5- | 1.44 |
| | fluoropentyl)-1H-indazole-3-carboxamide | |
| 292 | N-[(1S)-1-(aminocarbonyl)-2-methylpropyl]-1-(5- | 1.43 |
| | fluoropentyl)-1H-indazole-3-carboxamide | |
| 293 | N-(1-amino-3-methyl-1-oxobutan-2-yl)-1-(4- | 1.43 |
| | fluoropentyl)-1H-indazole-3-carboxamide | |
| 294 | N-(naphthalen-1-yl)-2-pentyl-2H-indazole-3-carboxamide | 1.43 |
| 295 | 7-methoxy-2-methyl-1-[2-(4-morpholinyl)ethyl]-N- | 1.43 |
| | [(1S,2S,4R)-1,3,3-trimethylbicyclo[2.2.1]hept-2-yl]-1H- | |
| | indole-3-carboxamide | |
| 296 | 1-(2-fluoropentyl)-N-(naphthalen-1-y1)-1H-indole-3- | 1.43 |
| | caxboxamide | |
| 297 | N-[1-(aminocarbonyl)-2,2-dimethylpropyl]-1-(5- | 1.42 |
| | fluoropentyl)-1H-indole-3-carboxamide | |
| 298 | N-((2S,3S)-1-amino-3-methyl-1-oxopentan-2-yl)-1-(5- | 1.42 |
| | fluoropentyl)-1H-indazole-3-carboxamide | |
| 299 | 2-(4-cyanobutyl)-N-(2-phenylpropan-2-yl)-2H-indazole-3- | 1.42 |
| | carboxamide | |
| 300 | (6-methoxy-1-naphthalenyl)(1-pentyl-1H-indol-3-yl)- | 1.42 |
| | methanone | |
| 301 | rel-2-[(1S,3S)-3-hydroxycyclohexyl)]-5-(2-methylnonan- | 1.41 |
| | 2-yl)phenol | |
| 302 | N-cyclopropyl-11-(2-hexyl-5-hydroxyphenoxy)- | 1.40 |
| | undecanamide | |
| 303 | 1-[(E)-(3-pentyl-1H-inden-1-ylidene)methyl]-naphthalene | 1.39 |
| 304 | 4-[4-(1,1-dimethylheptyl)-2,6-dimethoxyphenyl]-6,6- | 1.39 |
| | dimethyl-bicyclo[3.1.1]hept-2-ene-2-methanol | |
| 305 | (1-(1-methylazepan-3-yl)-1H-indol-3-yl)(2,2,3,3- | 1.38 |
| | tetramethylcyclopropyl)methanone | |
| 306 | 1-(5-fluoropentyl-N-(phenylmethyl)-1H-indole-3- | 1.38 |
| | carboxamide | |
| 307 | 1-pentyl-N-phenyl-1H-indole-3-carboxamide | 1.38 |
| 308 | (4-methoxyphenyl)[2-methyl]-1-[2-(4-morpholinyl)ethyl]- | 1.38 |
| | 1H-indol-3-yl]-methanone | |
| 309 | N-((3s,5s,7s)-adamantan-1-yl)-1-(5-fluoropentyl)-1H- | 1.37 |
| | indazole-3-carboxamide | |
| 310 | [6-bromo-2-methyl-1-(2-(4-morpholinyl)ethyl]-1H-indol- | 1.37 |
| | 3-yl](4-methoxyphenyl)-methanone | |
| 311 | 1-(5-fluoropentyl)-8-quinolinyl ester-1H-indazole-3- | 1.36 |
| | carboxylic acid | |
| 312 | N-[(1S)-1-(aminocarbonyl)-2-methylpropyl]-1-(5- | 1.35 |
| | fluoropentyl)-1H-indole-3-carboxamide | |
| 313 | 1-(5-fluoropentyl)-N-(2-phenylpropan-2-yl)-1H- | 1.33 |
| | pyrrolo[2,3-b]pyridine-3-carboxamide | |
| 314 | quinolin-6-yl 1-(cyclohexylmethyl)-1H-indole-3- | 1.32 |
| | carboxylate | |
| 315 | naphthalen-1-yl(9-pentyl-9H-carbazol-3-yl)methanone | 1.32 |
| 316 | (1-(4-fluoropentyl)-1H-indol-3-yl)(naphthalen-1- | 1.32 |
| | yl)methanone | |
| 317 | N-(1-amino-3-methyl-1-oxobutan-2-yl)-1-(2- | 1.31 |
| | fluoropentyl)-1H-indazole-3-carboxamide | |
| 318 | (9-(5-fluoropentyl)-9H-carbazol-3-yl)(naphthalen-1- | 1.31 |
| | yl)methanone | |
| 319 | 1-(5-fluoropentyl)-N-tricyclo[3.3.1.13,7]dec-1-yl-1H- | 1.31 |
| | indole-3-carboxamide | |
| 320 | [6-iodo-2-methyl-1-[2-(4-morpholinyl)ethyl]-1H-indol-3- | 1.30 |
| | yl](4-methoxyphenyl)-methanone | |
| 321 | (S)-N-(1-amino-3-methyl-1-oxobutan-2-yl)-1-(5- | 1.29 |
| | fluoropentyl)-3-(4-fluorophenyl)-1H-pyrazole-5- | |
| | carboxamide | |
| 322 | N-(1-methyl-1-phenylethyl)-1-[(tetrahydro-2H-pyran-4- | 1.27 |
| | yl)methyl]-1H-indazole-3-carboxamide | |
| 323 | N-[(1S)-1-(aminocarbonyl)2-methylpropyl]-1- | 1.26 |
| | (phenylmethyl)-1H-indole-3-carboxamide | |
| 324 | (4-benzylpiperazin-1-yl)(1-(5-fluoropentyl)-1H-indol-3- | 1.26 |
| | yl)methanone, monohydrochloride | |
| 325 | (1-butyl-1H-indol-3-yl)(naphthalen-2-yl)-methanone | 1.26 |
| 326 | (4-methyl-1-naphthalenyl)(2-methyl-1-pentyl-1H-indol-3- | 1.25 |
| | yl)-methanone | |
| 327 | isoquinolin-6-yl 1-(cyclohexylmethyl)-1H-indole-3- | 1.24 |
| | carboxylate | |
| 328 | N-[(1-pentyl-1H-indol-3-yl)carbonyl]-L-valine, methyl | 1.24 |
| | ester | |
| 329 | N-1-naphthalenyl-1-pentyl-1H-indole-3-carboxamide | 1.23 |
| 330 | 1-(4-fluorobenzyl)-N-(naphthalen-1-yl)-1H-indole-3- | 1.23 |
| | carboxamide | |
| 331 | N-cyclopropyl-11-(3-hydroxy-5-pentylphenoxy)- | 1.22 |
| | undecanamide | |
| 332 | naphthalen-2-yl 1-(2-fluorophenyl)-1H-indazole-3- | 1.21 |
| | carboxylate | |
| 333 | N-((3s,5s,7s)-adamantan-1-yl)-1-(5-chloropentyl)-1H- | 1.21 |
| | indazole-3-carboxamide | |
| 334 | (4-methylnaphthalen-1-yl)(1-(pent-4-en-1-yl)-1H-indol-3- | 1.20 |
| | yl)methanone | |
| 335 | N-(1-amino-3-methyl-1-oxobutan-2-yl)-1-(3- | 1.20 |
| | fluoropentyl)-1H-indazole-3-carboxamide | |
| 336 | (1-(tert-butyl)-1H-indol-3-yl)(naphthalen-1-yl)methanone | 1.20 |
| 337 | 1-(5-fluoropentyl)-N-(naphthalen-2-y1)-1H-indole-3- | 1.19 |
| | carboxamide | |
| 338 | 1-(3-fluoropentyl)-N-(naphthalen-1-y1)-1H-indole-3- | 1.19 |
| | carboxamide | |
| 339 | 1-(4-fluoropentyl)-N-(naphthalen-1-yl)-1H-indole-3- | 1.19 |
| | carboxamide | |
| 340 | isoquinolin-7-yl 1-(5-fluoropentyl)-1H-indole-3- | 1.18 |
| | carboxylate | |
| 341 | (1-(6-fluorohexyl)-1H-indol-3-yl)(naphthalen-1- | 1.18 |
| | yl)methanone | |
| 342 | (1-(3-methylbutan-2-yl)-1H-indol-3-yl)(naphthalen-2- | 1.17 |
| | yl)methanone | |
| 343 | (S)-N-(1-amino-3-methyl-1-oxobutan-2-yl)-2- | 1.14 |
| | (cyclohexylmethyl)-2H-indazole-3-carboxamide | |
| 344 | (S)-N-(1-amino-3-methyl-1-oxobutan-2-yl)-1- | 1.14 |
| | (cyclohexylmethyl)-5-(4-fluorophenyl)-1H-pyrazole-3- | |
| | carboxamide | |
| 345 | N-[(1S)-1-(aminocarbonyl)-2,2-dimethylpropyl]-2,3- | 1.13 |
| | dihydro-3-(3-hydroxy-3-methylbutyl)-2-oxo-1H- | |
| | benzimidazole-1-carboxamide | |
| 346 | (5-methoxynaphthalen-1-yl)(1-pentyl-1H-indol-3- | 1.13 |
| | yl)methanone | |
| 347 | 1-(4-Methoxyphenyl) piperazine (hydrochloride) | 1.12 |
| 348 | N,β-dimethyl-1,3-benzodioxole-5-propanamine, | 1.11 |
| | monohydrochloride | |
| 349 | N-2-naphthalenyl-1-pentyl-1H-indole-3-carboxamide | 1.10 |
| 350 | (1-(sec-butyl)-1H-indol-3-yl)(naphthalen-2-yl)-methanone | 1.08 |
| 351 | N-(1-methyl-1-phenylethyl)-1-pentyl-1H-indole-3- | 1.07 |
| | carboxamide | |
| 352 | N-(adamantan-2-yl)-1-((tetrahydro-2H-pyran-4- | 1.07 |
| | yl)methyl)-1H-indazole-3-carboxamide | |
| 353 | rel-5-(1,1-dimethylheptyl-2-[(1R,3S)-3- | 1.06 |
| | hydroxycyclohexyl]-phenol | |
| 354 | N-[[1-(5-fluoropentyl)-1H-indol-3-yl]carbonyl]-L-valine, | 1.05 |
| | methyl ester | |
| 355 | N-(1-amino-3S-methyl-1-oxopentan-2S-yl)-2-(5- | 1.05 |
| | fluoropentyl)-2H-indazole-3-carboxamide | |
| 356 | 1-(5-fluoropentyl)-N-(1-methyl-1-phenylethyl)-1H-indole- | 1.03 |
| | 3-carboxamide | |
| 357 | N-(cyclopropylmethyl)-1-(5-fluoropentyl)-1H-indole-3- | 1.03 |
| | carboxamide | |
| 358 | (3s,5s,7s)-adamantan-1-yl 1-(5-fluoropentyl)-1H-indazole- | 1.02 |
| | 3-carboxylate | |
| 359 | (1-(2-fluoropentyl)-1H-indol-3-yl)(4-methylnaphthalen-1- | 1.00 |
| | yl)methanone | |
| 360 | 3-(1,1-dimethylheptyl)-6aS,7,10,10aS-tetrahydro-1- | 0.96 |
| | hydroxy-6,6-dimethyl-6H-dibenzo[b,d]pyran-9-methanol | |
| 361 | 1-[(tetrahydro-2H-pyran-4-yl)methyl]-N- | 0.95 |
| | tricyclo[3.3.1.1^{3,7}]dec-1-yl-1H-indazole-3-carboxamide | |
| 362 | naphthalen-2-yl(1-(pentan-2-yl)-1H-indol-3-yl)methanone | 0.95 |
| 363 | 1-(cyclohexylmethyl)-2-[(4-ethoxyphenyl)methyl]-N,N- | 0.92 |
| | diethyl-1H-benzimidazole-5-carboxamide | |
| 364 | 1,4-dihydro-6-(1-methylethy1)-4-oxo-1-pentyl-N- | 0.89 |
| | tricyclo[3.3.1.1^{3,7}]dec-1-yl-3-quinolinecarboxamide | |
| 365 | 2-((1S,2S,5S)-5-hydroxy-2-(3-hydroxypropyl)cyclohexyl)- | 0.89 |
| | 5-(2-methyloctan-2-yl)phenol | |
| 366 | N,N-diethyl-2-(1-pentyl-1H-indo1-3-yl-4- | 0.89 |
| | thiazolemethanamine | |
| 367 | 6aR,7,10,10aR-tetrahydro-6,6,9-trimethyl-3-(1-methyl-1- | 0.87 |
| | phenylethyl)-6H-dibenzo[b,d]pyran-1-ol | |
| 368 | [1-[(1-methyl-2-piperidinyl)methyl]-1H-indol-3- | 0.85 |
| | yl](2,2,3,3-tetramethylcyclopropyl)-methanone | |
| 369 | N-(2-methoxyethyl)-N-(1-methylethyl)-2-(1-pentyl-1H- | 0.85 |
| | indol-3-yl)-4-thiazolemethanamine, monohydrochloride | |
| 370 | (1-butyl-1H-indol-3-yl)(4-methoxy-1-naphthalenyl)- | 0.82 |
| | methanone | |
| 371 | 2[(1S,3R)-3-hydroxycyclohexyl]-5-(2-methyloctan-2- | 0.78 |
| | yl)phenol | |
| 372 | (4-methoxy-1-naphthalenyl)(2-methyl-1-pentyl-1H-indol- | 0.60 |
| | 3-yl)-methanone | |
| 373 | 1-(5-fluoropentyl)-N-(naphthalen-1-yl)-1H-pyrrolo[3,2- | 0.49 |
| | c]pyridine-3-carboxamide | |
| 374 | cis-5-(1,1-dimethylnonyl)-2-(3-hydroxycyclohexyl)- | 0.49 |
| | phenol | |
| 375 | rel-2-((1R,2R,5S)-5-hydroxy-2-(3- | 0.33 |
| | hydroxypropyl)cyclohexyl)-5-(2-methyloctan-2-yl)phenol | |
| 376 | [2,3-dihydro-5-methyl-3-(4- | 0.32 |
| | morpholinylmethyl)pyrrolo(1,2,3-de]-1,4-benzoxazin-6- | |
| | yl]-1-naphthalenyl-methanone, methanesulfonate | |
| 377 | 3-hydroxy-2-[(1R,6R)-3-methyl-6-(1-methylethenyl)-2- | 0.30 |
| | cyclohexen-1-yl]-5-pentyl-2,5-cyclohexadiene-1,4-dione | |
| 378 | 3-(1,1′dimethylheptyl)-6aR,7,10,10aR-tetrahydro-1- | 0.21 |
| | hydroxy-6,6-dimethyl-6H-dibenzo(b,d]pyran-9-methanol | |
| 379 | [(3R)-2,3-dihydro-5-methyl-3-(4- | 0.21 |
| | morpholinylmethyl)pyrrolo[1,2,3-de]-1,4-benzoxazin-6- | |
| | yl]-1-naphthalenyl-methanone, methanesulfonate | |
| 380 | rel-5-(1,1-dimethylheptyl)-2-[(1R,2R,5R)-5-hydroxy-2-(3- | 0.17 |
| | hydroxypropyl)cyclohexyl]-phenol | |

### Example 3

### MHC-I Expression Restoration in Cancer Cells

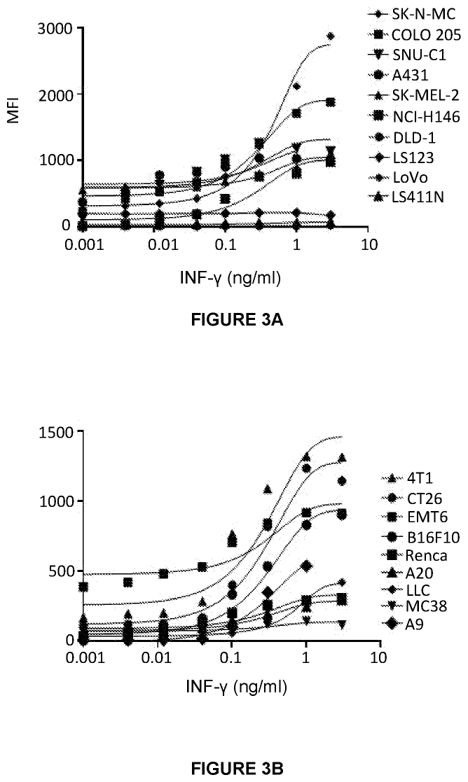

MHC-I expression may be restored in cancers, such as those with intact antigen processing machinery (APM) genes. Various human and mouse cancer cell lines were treated with dose titrations of recombinant human and mouse IFN-γ, respectively. The cells were incubated with IFN-γ for 48 hours in a humidified chamber at 37° C., stained with fluorescent haplotype-appropriate MHC-I antibody, then signal was determined by flow cytometry. Cancers represented in this experiment include brain (SK-N-MC), breast (4T1, EMT6), colorectal (COLO 205, SNU-C1, DLD-1, LS123, LS411N, LoVo, CT26, MC38), kidney (Renca), lung (NCI-H146, LLC), lymphoid (A20), and skin (A431, SK-MEL-2, B16F10). IFN-γ induced MHC-I expression in a dose-dependent manner in 6/10 (60%) human and 6/9 mouse (67%) cell lines. These numbers are consistent with the previously reported values and demonstrate that the APM is intact and can be induced in many cancers. The results are found in FIGS. 3A-3B.

### Example 4

### Phytocannabinoids Induced MHC-I Expression Restoration in Cancer Cells

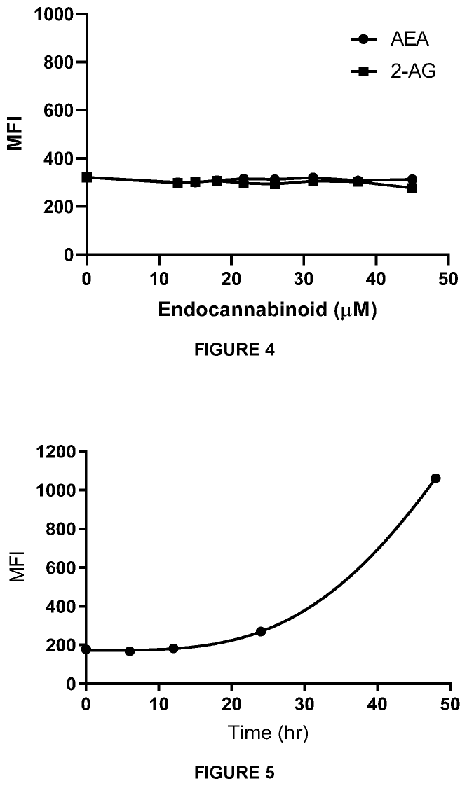

Phytocannabinoids were tested for MHC-I-inducing activity on COLO 205 cells. Each cannabinoid was tested at a concentration range of 0-100 μM. After a 48 hours incubation of cells with cannabinoid, MHC-I expression was determined by flow cytometry using the pan-HLA antibody W6/32 (Thermo Fisher). The MHC-I induction results of the 15 cannabinoids are compiled in Table 5. The phytocannabinoids induced MHC-I expression to varying levels, with seven achieving ≥3-fold induction relative to the control cells treated only with buffer and incubated under the same conditions. Of the phytocannabinoids tested, cannabidiol (CBD) was the most potent, with an EC₅₀ of 10 μM. “ND” indicates not determined. In contrast, endogenous cannabinoids, represented by AEA (N-arachidonoylethanolamine; anandamide) and 2-AG (2-Arachidonoylglycerol) do not induce MHC-I expression in COLO 205 cells. The results are found in FIG. 4.

TABLE 5
Induction of MHC-I expression on COLO
205 cells by phytocannabinoids.

| Example | Cannabinoid | Fold Induction | EC₅₀ (μM) |
|---|---|---|---|
| 1 | Cannabigerol | 2.7 | 40 |
| 2 | Cannabidiol | 2.8 | 10 |
| 3 | Cannabichromene | 1.6 | 20 |
| 4 | Cannabinol | 1.8 | 25 |
| 5 | Tetrahydrocannabivarin | 3.0 | 15 |
| 381 | Cannabichromenevarin | 3.3 | 32 |
| 382 | Cannabicitran | 1.3 | ND |
| 383 | Δ8-THC | 4.1 | 27 |
| 384 | Δ9-THC | 6.1 | 30 |
| 385 | Cannabivarin | 3.9 | 20 |
| 386 | Cannabidivarin | 5.5 | 19 |
| 387 | CBN Monomethyl Ether | 2.3 | 30 |
| 388 | Cannabigerorcin | 2.7 | 36 |
| 389 | Cannabigerorcinic acid | 2.6 | ND |
| 390 | Cannabicyclol | 4.2 | 20 |

### Example 5

### MHC-I Up-Regulation in COLO 205 Cancer Cells

To determine whether changes in gene expression play a role in cannabinoid-induced MHC-I expression, COLO 205 cells were incubated with 15 μM CBD and cells were harvested at 6, 12, 24, and 48 hours for determination of MHC-I expression by flow cytometry. As shown in FIG. 5, a slight increase in MHC-I expression was observed by 24 hours, but robust induction was not observed until 48 hr. The long lag period between the stimulus and the response suggests that changes in gene expression is involved in MHC-I upregulation.

### Example 6

### MHC Class I Upregulation with Cannabigerol Treated A9 Metastatic Tumor Cells

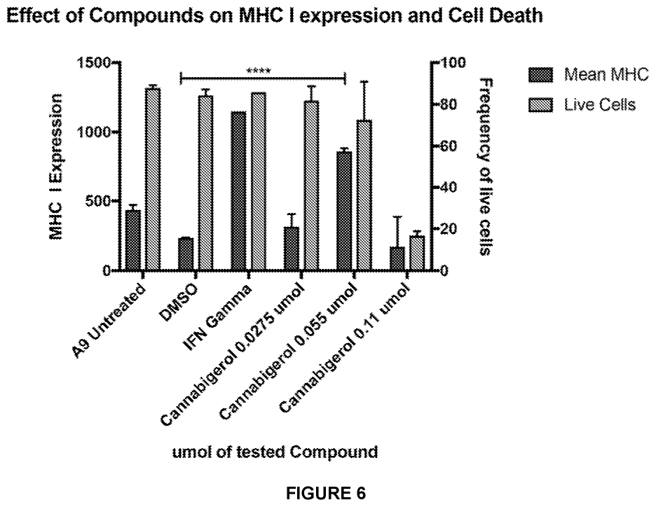

The effect of cannabigerol on MHC class I in A9 cancer cells were determined as follows. A9 cells (1×10⁶) were plated onto a 6 well plate (Corning) in two mL of media. A9 cells were treated after 24 hours at various concentrations of Cannabigerol, 5.832×10⁴ nM IFN gamma or DMSO vehicle for 48 hours. Cells were then harvested from the plate as per protocol and half of the cells were checked for MHC class I expression levels using flow cytometry. The Anti-mouse H2 KB APC antibody (Biolegend) was used at 1:200 in FACS buffer (2% FBS in PBS). FIG. 6 demonstrates that there is a significant increase in class I MHC upon treatment with Cannabigerol (0.055 μM) relative to that of the vehicle (DMSO) treated cells, with a p value of <0.0001 while using an ordinary one-way ANOVA. This indicates that Cannabigerol elicits a similar response in the tumor environment as IFN-γ, which is known to recruit innate immune cells and activate cytolytic activity of NK cells and macrophages. FIG. 6 also demonstrates cell death at 0.11 μM. At a concentration of 0.0275 μM, there was very little cell death; the level of cell death was comparable to that of DMSO treated or untreated cells. However, at this concentration there was less upregulation of class I MHC relative to DMSO-treated or untreated A9 cells.

### Example 7

### Cannabigerol Causes T-Cell Activation

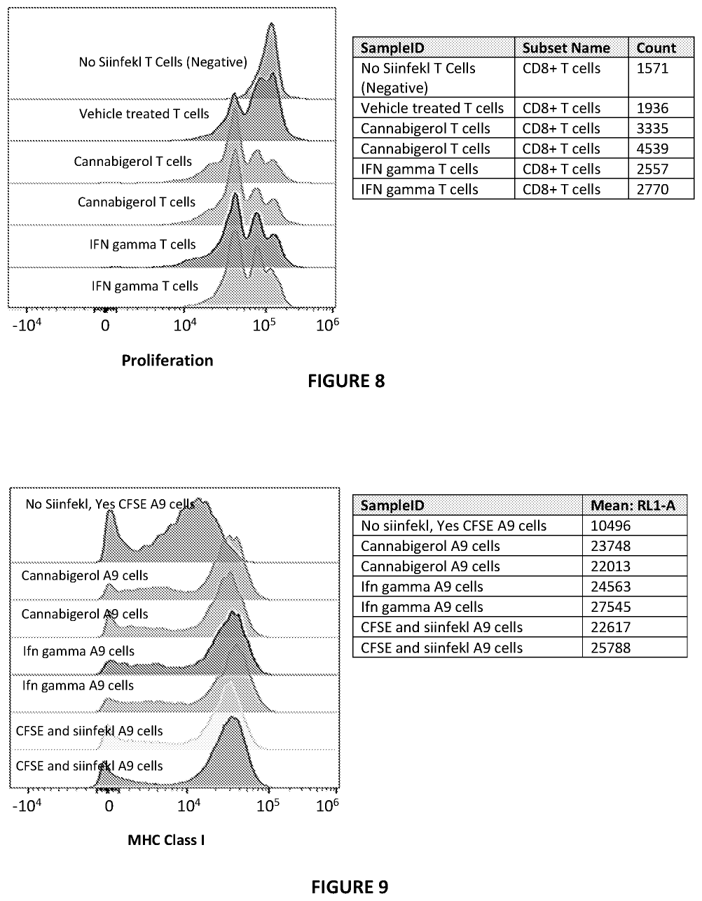

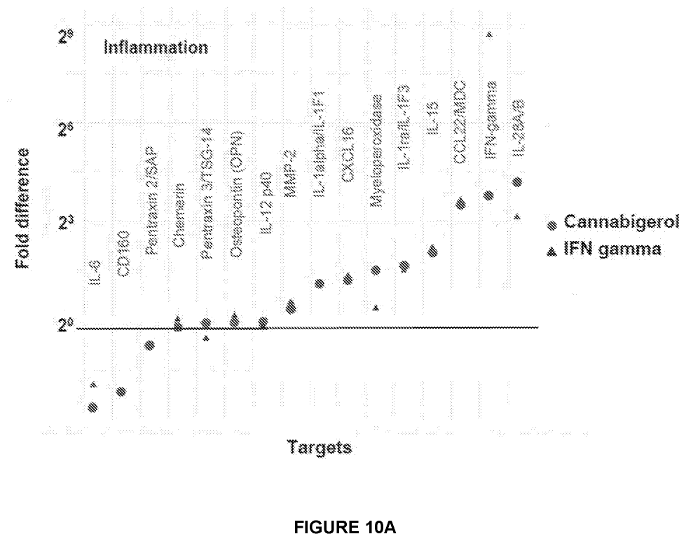

A cytolytic assay was performed as follows. A9 cells (1×10⁶) were plated onto a 6 well plate in two mL of RPMI media (Advanced RPMI-1640 Medium (Gibco), 100 U/mL of Penicillin-Streptomycin (P+S) (Thermofisher), 1% L-Glutamine (Gibco), and 10% FBS). A9 cells were treated with either Cannabigerol 0.055 μM, 5.832×10⁴ nM IFN gamma, or 1% DMSO vehicle. After 24 hours, SIINFEKL OVA Peptide (SIINFEKL) (Genscript), was added to the A9 cells. Following an additional 24 hours, CD8⁺ T cells were collected from OT1 mouse spleens. Spleens were collected and passed through a 100 micron cell strainer (Falcon). Red blood cells were removed from the spleen isolate using Red Blood Cell ACK lysis buffer (Gibco). Enrichment for CD8⁺ T cells was done using CD8⁺ Untouched Mouse CD8⁺ cells Dynabeads (Thermofisher), as per the manufacturer's protocol. RPMI media was removed from the A9 cells, and A9 cells were washed three times with PBS before being replaced with RPMI media. An extra well of untreated A9 cells was counted and used as a standard count. CD8⁺ T cells were counted and then treated with CFSE (Biolegend) per manufacture protocol, before being co-cultured with A9 cells to a 1:1 ratio or a 1:5 ratio. T cells for the positive control were stimulated 24 hours later using CD28 Monoclonal Antibody clone 37.51 (eBioscience) at a concentration of 5 μg/mL and CD3e Monoclonal Antibody clone 145-2C11 at 10 μg/mL, (eBioscience). CD8+ T cells were collected from the cell media, and spun down at 1500 RPMI for use with FACS, and A9s were harvested for FACS. CD8+ T cells were stained with CD8 PE-efluor 610 antibody (Table 6). A9 Cells were stained using PE H-2 KB antibody (Table 6), and 7AAD viability dye (Table 6) in FACS buffer. Ex vivo, OT1 (ova-peptide SIINFEKL specific) CD8⁺ T cells were found to show increased proliferation when their co-cultured metastatic tumor cells were treated with Cannabigerol for 48 hours prior to co-culture, suggesting that the T cells are activated and would have increased cytolytic T cell activity upon use of Cannabigerol (FIG. 8). There was an increase in the MHC class I on the A9 cells associated with the OT1 T cells (FIG. 9).

TABLE 6
Antibody panel used for FACS during mouse tumor trial

| | Fluorophore | | | Ratio |
|---|---|---|---|---|
| Target | (Anti-mouse) | Clone | Company | used |
| CD3E | APC-eFluor 780 | 145-2C11 | Invitrogen | 1:200 |
| CD11C | eFluor 506 | N418 | Invitrogen | 1:200 |
| CD4 | Alexafluor 700 | GK1.5 | Invitrogen | 1:200 |
| CD69 | PE-Cyanine7 | H1.2 F3 | Invitrogen | 1:200 |
| CD45R | FITC | RA3-6B2 | Invitrogen | 1:200 |
| MHCII | eFluor 450 | AF6-120.1 | Invitrogen | 1:200 |
| CD49B | APC | DX5 | Invitrogen | 1:200 |
| CD11B | SuperBright 645 | M1/70 | Invitrogen | 1:200 |
| CD62L | SuperBright 702 | MEL-14 | Invitrogen | 1:200 |
| MHC Class I | PE | AF6-88.5.5.3 | Invitrogen | 1:200 |
| (H-2KB) | | | | |
| Viability | PerCP | — | Biolegend | 2:200 |
| Dye 7AAD | Cyanine5.5 | | | |
| CD8A | PE-EF610 | 53-6.7 | Invitrogen | 1:200 |
| Super Bright | | — | eBioscience | 50:200 |
| Staining | | | | |
| Buffer | | | | |

### Example 8

### Mouse Model

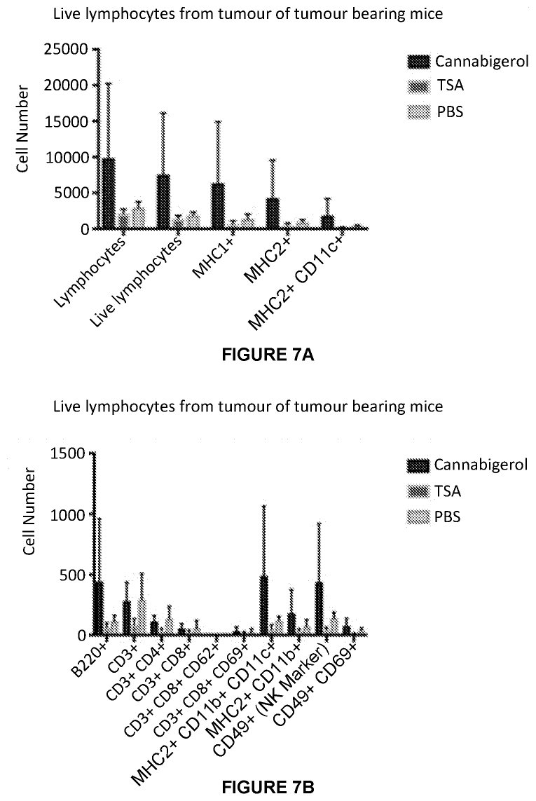

To confirm the in vivo activity of cannabinoid compounds, a representative cannabinoid was tested in a representative animal model. A9 tumor cells were injected subcutaneously into mice at 8-10 weeks of age, and mice were treated 7 days later with either Cannabigerol, TSA, or Vehicle. 1×10⁵ A9 cells were injected in a volume of 50 μL of PBS into the right flank of each mouse subcutaneously. Starting at day 7 after injection of the A9 tumor cells, intraperitoneal injections of Cannabigerol (Cayman Chemical Company) at 10 mg/kg, Trichostatin A (TSAX Sigma-Aldrich) at 0.5 mg/kg dissolved in 1% DMSO and PBS, or vehicle (0.2% DMSO, 5% Bovine Serum Albumin (BSA), in PBS) were administered daily to the animals. Cannabigerol was dissolved in 0.2% DMSO, 5% Bovine Serum Albumin, in PBS. Animals were weighed 3 times a week and during tumor measurements, and tumor measurements were begun upon appearance of the tumor, on days 12 and 13 of the trial. Tumor volume was determined using calipers to measure tumor dimensions. Mice were sacrificed at day 10, 11, and 12, as was dictated by subscribed humane endpoints, in which mice experienced tumor ulceration. Half of the tumors were removed from the mouse and immediately placed in digestion media (RPMI Medium 1640 (Gibco) with 10% FBS and Penicillin+Streptomycin (P+S), with the addition of 10% collagenase hyaluronidase, 15% DNAse I (Stemcell)). Tumors were chopped in 1 mL of digestion media and placed at a temperature of 35° C. for 30 minutes, before being passed through a 100 micron cell strainer (Falcon). Cells from these tumors were stained in FACS buffer using the antibody panel from Table 6, before being submitted to flow cytometry on the Attune 2 FACS machine. The second half of the tumors were placed in Neg-50 (Richard Allan Scientific) for freezing and further histology. The lungs, adrenal glands, and liver were collected and placed in formalin for further histology. All spleens were collected and passed through a 100 micron cell strainer. Mouse weight and tumor volume appeared similar among Cannabigerol treated and Vehicle treated groups. Much higher absolute numbers of lymphocytes were observed in the Cannabigerol treated mouse tumors relative to the vehicle treated tumors (FIGS. 7A-7B).

### Example 9

### Cytokine Secretion Profile in Cannabigerol Treated A9 Cells

Cannabigerol and IFN gamma were dissolved in 1% Dimethyl Sulfoxide (DMSO) (Sigma) in media (1% DMSO). 1×10⁶ A9 cells were plated onto a 6 well plate in two mL of DMEM media. Twenty-four hours after seeding, cells were cultured at the optimum concentrations of Cannabigerol 0.055 μM or 5.832×10⁻⁶ nM IFN gamma or 1% DMSO vehicle for 48 hours. Relative expression levels of 111 soluble mouse proteins including cytokines, chemokines and growth factors were evaluated using the Proteome Profiler Mouse XL cytokine array kit (R&D System, ARY028) following the manufacturer's instructions. Spot densities on the array film were detected and quantified using Image J analysis software Image J protein analyzer add-on on a scanned version of the film. Quantification of the spot intensity in the arrays was conducted with background subtraction in ImageJ. To determine fold change, the treatment values of IFN gamma microarrays were divided by the DMSO negative control values of the concurring spots. A value of 1 was subtracted from the absolute value of this fold change, to correct the value of DMSO to “0”. Experiment was done in technical replicates. Cytokines that showed changes in expression levels were further characterized by pathway analysis for over-represented pathway identification through Reactome Database release 65, Pathway Brower Version 3.5.

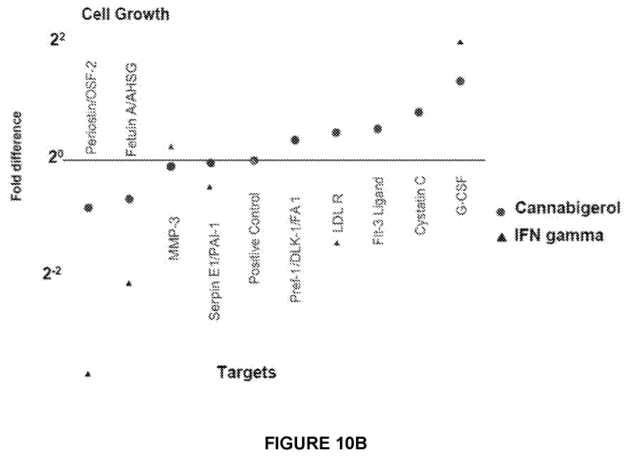

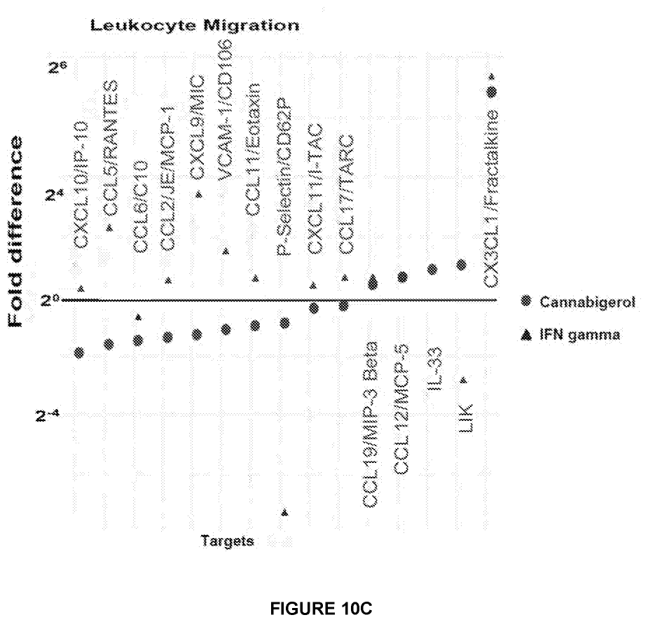

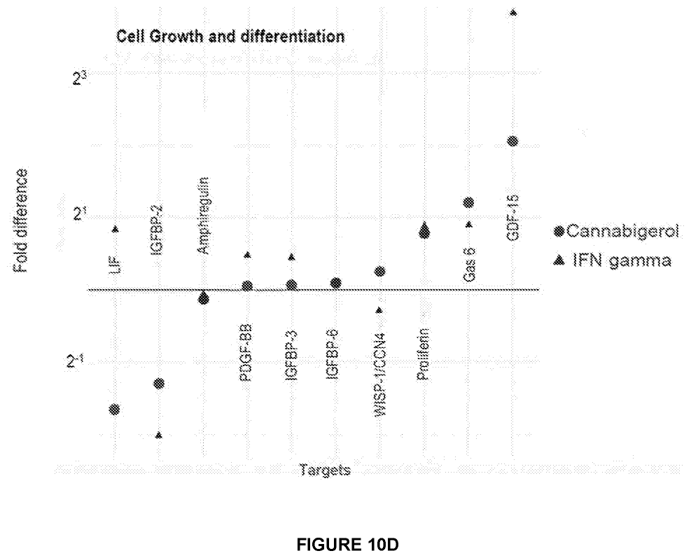

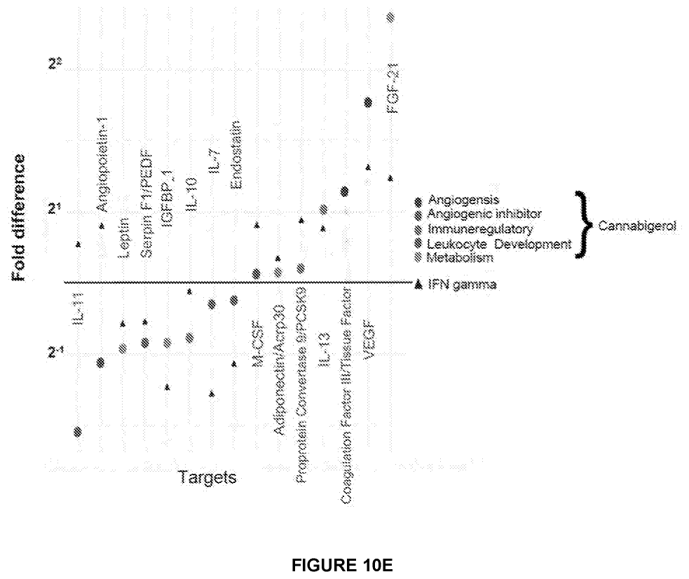

Cannabigerol was found to cause a change in cytokines involved in inflammation, migration, growth and differentiation, angiogenesis, immune regulation, leukocyte development and metabolism (FIGS. 10A-10E). Cytokines that were upregulated upon use of cannabigerol, included FGF-21 and IL-13. There also was an increase in VEGFA. Additionally, there was downregulation of IL-10, and IL-11. There also was a decrease in the production of IGFBP1 and angiopoietin-1,

All of the articles and methods disclosed and claimed herein can be made and executed without undue experimentation in light of the present disclosure. While the articles and methods of this disclosure have been described in terms of preferred embodiments, it will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure as defined by the appended claims. All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes. The disclosure illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

1. A method for increasing major histocompatibility complex class I (MHC-1) surface expression in a cell, comprising contacting the cell with a cannabinoid compound, or derivative thereof.

2. The method according to claim 1, wherein the cell is a cancer cell.

3. The method according to claim 1, wherein the cell is infected by an intracellular pathogen.

4-8. (canceled)

9. A method for enhancing an immune response against cancer, comprising administering to a subject a pharmaceutically effective amount of a cannabinoid compound or derivative thereof and a suitable adjuvant or carrier, in preparation of a medicament.

10. (canceled)

11. A method for enhancing an immune response against a disease caused by an intracellular pathogen, comprising administering to a subject a pharmaceutically effective amount of a cannabinoid compound or derivative thereof and a suitable adjuvant or carrier, in preparation of a medicament.

12. A method according to claim 11, where the disease caused by the intracellular pathogen is a viral, bacterial, or fungal infection.

13. A method according to claim 12, wherein the cannabinoid compound or derivative thereof and a suitable adjuvant or carrier is combined with the use of an antiviral or antimicrobial therapy.

14-18. (canceled)

19. The method of claim 1 wherein the cannabinoid compound or derivative thereof is selected from cannabigerol, cannabidiol, cannabichromene, cannabinol, tetrahydrocannabivarin, cannabichromevarin, cannabicitran, Δ8-THC, Δ9-THC, cannabivarin, cannabidivarin, CBN Monomethyl Ether, cannabigerorcin, cannabigerorcinic acid and cannabicyclol.

20. The method of claim 1, wherein said method further comprises administering an additional therapeutic agent to said subject.

21. The method of claim 20, wherein said additional therapeutic agent is:
a. an immune activating agent;
b. checkpoint inhibitor;
c. co-activating receptor agonist;
d. a cancer vaccine;
e. adoptive cell therapy, or a T-cell expressing a chimeric antigen receptor or a T-cell expressing a modified T-cell receptor;
f. a cytokine, IL-2, IFN-alpha, or BCG; or
g. a cytotoxic agent, cytostatic agent or a combination of cytotoxic agents and cytostatic agents.

22-25. (canceled)

26. The method of claim 21, wherein said additional therapeutic agent is selected from CTLA inhibitors, PD-1 pathway inhibitors, anti-OX40 antibodies, vaccines, oncolytic virus, CAR T cells, or a combination thereof.

27-29. (canceled)

30. The method of claim 9 wherein the subject has cancer that is resistant to one or more cytotoxic agents.

31. The method of claim 9 wherein the subject has stage III or stage IV cancer.

32. The method of claim 9 wherein the subject has metastatic cancer.

33. The method of claim 1 wherein the cannabinoid compound is administered orally or sublingually.

34. The method of claim 33 wherein the cannabinoid compound is administered in a unit dose form.

35-36. (canceled)

37. The method of claim 1 wherein the cannabinoid compound is administered orally, topically, IV, subcutaneously or sublingually at a dose ranging from 1 to 50 mg/kg/day, preferably 5-40 mg/kg/day or 10-20 mg/kg/day.

38-42. (canceled)

43. A method according to claim 1, wherein said cancer is not characterized by dysregulation of the IL-10 and/or GM-CSF pathways in cancerous cells.

44. The method according to claim 1 comprising contacting the cell with a compound of Formula I, or a pharmaceutically acceptable salt thereof; 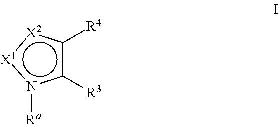
wherein X¹ is —CR⁵—, nitrogen or —NR⁵—;
wherein X² is —CR²—, nitrogen or —NR⁵—; provided X² is not nitrogen or —NR⁵— if X is nitrogen or —NR⁵—;
wherein R¹ is absent, H, or R¹;
wherein R¹ is selected from alkyl, haloalkyl, cyanoalkyl, alkenyl, heterocyclyl, cycloalkylalkyl, heterocyclylalkyl, arylalkyl and aryl;
wherein R² is selected from H, aryl, aminocarbonylalkylaminocarbonyl, alkoxycarbonylalkylaminocarbonyl, aminocarbonyl(arylalkyl)aminocarbonyl, alkenylcarbonyl, arylcarbonyl, cycloalkylcarbonyl, arylalkylcarbonyl, heterocyclylalkylcarbonyl, heterocyclylcarbonyl, alkoxycarbonylalkyloxycarbonyl, cycloalkyloxycarbonyl, aryloxycarbonyl, heterocyclyloxycarbonyl, arylaminocarbonyl, arylalkylaminocarbonyl, heterocyclylaminocarbonyl, cycloalkylaminocarbonyl, optionally substituted arylalkyl and heterocyclylcarbonylalkyl;
wherein R³ is H or aryl;
wherein R⁴ is H or amino;
wherein R⁴ and R³ together form a 6-membered aryl or heteroaryl ring, wherein the ring is optionally substituted with one or more substituents selected from halo, nitro, C₁₋₆-alkoxy, alkylaminocarbonyl, or optionally substituted C₆₋₁₀ aryl; and
R⁵ is selected from H, alkyl, arylcarbonyl, aminocarbonylalkylaminocarbonyl, arylalkyl, haloalkyl, cycloalkylalkyl, and cyanoalkyl; or a pharmaceutically acceptable salt thereof.

46-75. (canceled)

76. The method of claim 9 for the treatment of patients with cancers of the epithelium, colon, brain, breast, kidney, lung, hematologic cells and skin.

77. (canceled)
